(19) United States
(12) Patent Application Publication
(10) Pub. No.: US 2024/0024089 A1
(43) Pub. Date: Jan. 25, 2024

(54) METHODS AND DEVICES FOR PROVIDING OXYGEN TO ENCAPSULATED CELLS

(71) Applicant: CORNELL UNIVERSITY, Ithaca, NY (US)

(72) Inventors: Longhai WANG, Ithaca, NY (US); Alexander ERNST, Ithaca, NY (US); Minglin MA, Ithaca, NY (US); James FLANDERS, Ithaca, NY (US)

(21) Appl. No.: 18/256,441

(22) PCT Filed: Dec. 9, 2021
(86) PCT No.: PCT/US2021/062648

§ 371 Date: Jun. 8, 2023

Related U.S. Application Data
(60) Provisional application No. 63/123,307, filed on Dec. 9, 2020.

(51) Int. Cl. A61F 2/02; A61L 27/02; A61L 27/16; A61L 27/18; A61L 27/38; A61L 27/52; A61L 27/54

(52) U.S. Cl. CPC A61F 2/022; A61L 27/025; A61L 27/16; A61L 27/18; A61L 27/38; A61L 27/52; A61L 27/54; A61F 2002/0081

(57) ABSTRACT

The present disclosure is directed to devices and systems for oxygenating encapsulated cells. The disclosure further relates to individual components of these devices and systems and methods of using the devices and systems to deliver a therapeutic agent to a subject in need thereof.

This application claims the priority benefit of U.S. Provisional Patent Application Ser. No. 63/123,307, filed Dec. 9, 2020, which is hereby incorporated by reference in its entirety.

This invention was made with government support under 1R01DK105967-01A1 awarded by National Institutes of Health and DGE-1650441 awarded by National Science Foundation. The government has certain rights in the invention.

### FIELD OF THE INVENTION

The present invention relates to devices and systems suitable for oxygenating encapsulated cells.

### BACKGROUND OF THE INVENTION

The transplantation of immunoprotected therapeutic-secreting cells has promised to provide a compliance- and immunosuppression-free cell replacement therapy for many hormone deficient diseases and endocrine disorders such as type I diabetes (T1D) (Ernst et al., “Islet Encapsulation,” J. Mater. Chem. B 6:6705-6722 (2018); Scharp and Marchetti, “Encapsulated Islets for Diabetes Therapy: History, Current Progress, and Critical Issues Requiring Solution,” Adv. Drug Del. Rev. 67:35-73 (2014); Lee and Bae, “Cell Transplantation for Endocrine Disorders,” Adv. Drug Del. Rev. 42:103-120 (2000)). T1D affects millions of people worldwide and currently does not have a cure (Atkins et al., “Type 1 Diabetes.” The Lancet 383:69-82 (2014)). Cell encapsulation technology intends to regulate blood glucose (BG) levels autonomously and prevent immune destruction of transplanted donor islets or stem cell-derived insulin-producing cells from the host by use of a semipermeable material that prevents graft interaction with immune effector cells.

It has been well documented that a critical constraint of cell replacement therapy is insufficient oxygen (O₂) supply (Colton, “Oxygen Supply to Encapsulated Therapeutic Cells,” Adv. Drug Del. Rev. 67:93-110 (2014) and Weaver et al., “Design of a Vascularized Synthetic Poly (Ethylene Glycol) Macroencapsulation Device for Islet Transplantation,” Biomaterials 172:54-65 (2018)). Pancreatic islets—cell clusters comprising glucose-sensing, insulin-secreting β cells and other secretory cells—are densely vascularized in their native state due to the high O₂ demand of insulin secretion (Bowers et al., “Engineering the Vasculature for Islet Transplantation,” Acta Biomater. 95:131-151 (2019). However, they are dissociated from arterial blood following isolation and encapsulation. This limits O₂ delivery to slow passive diffusion from extra-arterial sources in the transplantation site, which are comparatively low in oxygen tension/availability (Colton, “Oxygen Supply to Encapsulated Therapeutic Cells,” Adv. Drug Del. Rev. 67:93-110 (2014); Carlsson et al., “Markedly Decreased Oxygen Tension in Transplanted Rat Pancreatic Islets Irrespective of the Implantation Site,” Diabetes 50:489-495 (2001); Carreau, “Why is the Partial Oxygen Pressure of Human Tissues a Crucial Parameter? Small Molecules and Hypoxia,” J. Cell. Mol. Med. 15:1239-1253 (2011); and Bochenek et al., “Alginate Encapsulation as Long-Term Immune Protection of Allogeneic Pancreatic Islet Cells Transplanted Into the Omental Bursa of Macaques,” Nat. Biomed. Eng. 2:810-821 (2018)). In particular, the partial pressure of O₂ (pO₂) experienced by a native β cell in the pancreas is roughly 40-60 mmHg (Carlsson et al., “Markedly Decreased Oxygen Tension in Transplanted Rat Pancreatic Islets Irrespective of the Implantation Site,” Diabetes 50:489-495 (2001)), whereas it is likely below 25 mmHg for cells in a transplanted islet (Carlsson et al., “Markedly Decreased Oxygen Tension in Transplanted Rat Pancreatic Islets Irrespective of the Implantation Site,” Diabetes 50:489-495 (2001) and Moore et al., “Bioengineered Stem Cells as an Alternative for Islet Cell Transplantation,” World J. Transplant. 5:1 (2015)). Hydrogel encapsulation further exacerbates this issue by increasing the O₂ diffusion distances to the cell clusters (Lewis, “Eliminating Oxygen Supply Limitations for Transplanted Microencapsulated Islets in the Treatment of Type 1 Diabetes,” Thesis, Massachusetts Institute of Technology (2008)), and the deposition of a fibrotic capsule around the graft during the foreign body reaction (Vlahos and Sefton, “Muted Fibrosis Form Protected Islets,” Nat. Biomed. Eng. 2:791-792 (2018)) often adds yet another mass transfer resistance limiting cellular O₂ availability. The subcutaneous (SC) space is one of the most clinically desirable transplantation sites due to its minimally invasive accessibility. But it is particularly O₂ limited and produces high levels of fibrotic deposition following material implantation (Carreau, “Why is the Partial Oxygen Pressure of Human Tissues a Crucial Parameter? Small Molecules and Hypoxia,” J. Cell. Mol. Med. 15:1239-1253 (2011) and Kastellorizios et al., “Foreign Body Reaction to Subcutaneous Implants,” Immune Responses to Biosurfaces 93-108 (2015)).

O₂ limitations impact both islet survival and metabolic function and possibly increase immunogenicity. Steep pO₂ gradients within isolated islets restrict O₂ flow to the islet core. This central hypoxia has deleterious consequences: at a pO₂ of ˜8 mmHg, β cell insulin secretion is substantially arrested (Avgoustiniatos, “Oxygen Diffusion Limitations in Pancreatic Islet Culture and Immunoisolation,” (2003)), and at levels below ˜0.08 mmHg, islet cells undergo programmed and unprogrammed cell death (Avgoustiniatos, “Oxygen Diffusion Limitations in Pancreatic Islet Culture and Immunoisolation,” (2003) and Moritz et al., “Apoptosis in Hypoxic Human Pancreatic Islets Correlates with HIF-1α Expression,” The FASEB Journal 16:745-747 (2002)). Such apoptotic and necrotic cells release danger-associated molecular patterns to which the host mounts an immune response, increasing the recruitment of immune cells to the graft (Sachet et al., “The Immune Response to Secondary Necrotic Cells,” Apoptosis 22:1189-1204 (2017) and de Vos et al., “Polymers in Cell Encapsulation from an Enveloped Cell Perspective,” Adv. Drug Del. Rev. 67:15-34 (2014)). This phenomenon may aggravate O₂ limitations by increasing O₂ depletion at the graft-host interface, thereby reducing the amount available for the encapsulated cells (Avgoustiniatos and Colton, “Effect of External Oxygen Mass Transfer Resistances on Viability of Immunoisolated Tissue A,” Ann. N. Y. Acad. Sci. 831:145-166 (1997)). Hypoxia significantly impairs the metabolic responsiveness of encapsulated islets and may also precipitate a positive feedback loop of worsening graft oxygenation and immunogenicity.

One of the most straightforward ways to address inadequate oxygenation in cell therapies is to directly inject O₂ into the encapsulation device. Indeed, animal studies (Barkai et al., “Enhanced Oxygen Supply Improves Islet Viability in a New Bioartificial Pancreas,” Cell Transplant. 22:1463-1476 (2013); Ludwig et al., “A Novel Device for Islet Transplantation Providing Immune Protection and Oxygen Supply,” Horm. Metab. Res. 42:918-922 (2010); Neufeld et al., “The Efficacy of an Immunoisolating Membrane System for Islet Xenotransplantation in Minipigs,” PLoS One 8:e70150 (2013); and Ludwig et al., “Favorable Outcome of Experimental Islet Xenotransplantation Without Immunosuppression in a Nonhuman Primate Model of Diabetes,” Proc. Natl. Acad. Sci. U.S.A. 114:11745-11750 (2017)) and a preliminary human trial (Ludwig et al., “Transplantation of Human Islets Without Immunosuppression,” Proc. Natl. Acad. Sci. U.S.A,” 110:19054-19058 (2013)) clearly showed the benefit of O₂ supplementation despite the requirement of tedious daily injections. In situ chemical O₂ generation is an emerging biomaterials strategy to supply O₂ without human intervention (Gholipourmalekabadi et al., “Oxygen-generating Biomaterials: A new, Viable Paradigm for Tissue Engineering?” Trends Biotechnol. 34:1010-1021 (2016)). Several inorganic peroxides spontaneously release O₂ or hydrogen peroxide (which decomposes to yield O₂) in aqueous environments. For example, sodium percarbonate ((Na₂CO₃)₂·1.5H₂O₂) and calcium peroxide (CaO₂) provided short-term (1-10 d) O₂ supply following their incorporation in degradable scaffolds (Harrison et al., “Oxygen Producing Biomaterials for Tissue Regeneration,” Biomaterials 28:4628-4634 (2007) and Oh et al., “Oxygen Generating Scaffolds for Enhancing Engineered Tissue Survival,” Biomaterials 30:757-762 (2009)). Pedraza et al. extended the duration of O₂ generation in such constructs to approximately one month by embedding CaO₂ particulates within a polydimethylsiloxane (PDMS) disk, which slowed the production rate by introducing a diffusional barrier between the water and the reactive particulates (Pedraza et al., “Preventing Hypoxia-induced Cell Death in Beta Cells and Islets Via Hydrolytically Activated, Oxygen-Generating Biomaterials,” Proc. Natl. Acad. Sci. U.S.A. 109:4245-4250 (2012)). This construct reduced hypoxic effects in encapsulated β cells transplanted intraperitoneally in mice, demonstrating that enhanced oxygenation improved graft outcomes and reduced the expression of immunostimulatory factors (Coronel et al., “Oxygen Generating Biomaterial Improves the Function and Efficacy of Beta Cells Within a Macroencapsulation Device,” Biomaterials 210:1-11 (2019)). However, water is not an optimal reactant for in vivo O₂ generation because its transport is difficult to regulate.

The present invention is directed to overcoming these and other deficiencies in the art.

### SUMMARY OF THE INVENTION

The present invention relates to an inverse-breathing encapsulation system for cell delivery.

A first aspect of the present invention relates to a reservoir device for providing oxygen to encapsulated cells. This reservoir device includes a reservoir suitable for enclosing a liquid, a liquid contained within the reservoir, where the liquid is permeable to gas, and an oxygen-generating compound, where the oxygen-generating compound is immersed in the liquid contained within the reservoir.

Another aspect of the present invention is directed to a cell encapsulation device. This cell encapsulation device includes a gas permeable membrane having proximal and distal ends, where the gas permeable member encloses an inner space. The inner space extends longitudinally between the proximal and distal ends of the gas permeable membrane, and the inner space is at least partially filled with air. The device further comprises a hydrogel layer covering an outer surface of the gas permeable membrane.

Another aspect of the present invention relates to a cell encapsulation device. This cell encapsulation device includes two or more elongated gas permeable membranes. Each membrane comprises proximal and distal ends and encloses an inner space that extends longitudinally between the proximal and distal ends of the gas permeable membrane and the inner space is at least partially filled with air. The proximal ends of the membrane include an opening and the distal ends of the membranes are sealed. The two or more gas permeable membranes of the device are twisted helically about a longest axis shared by the membranes. The device further comprises a hydrogel layer covering outer surfaces or the two or more helically twisted gas permeable membranes.

Another aspect of the present invention includes a system for providing oxygen to encapsulated cells. The system includes the reservoir device as described herein and a cell encapsulation device as described herein.

Another aspect of the present invention includes a method of delivering a therapeutic agent to a subject in need thereof. The method of delivery includes obtaining a cell encapsulation device or system as described herein, and implanting the cell encapsulation device or system in the subject.

Described herein is a novel O₂ generating system regulated by carbon dioxide (CO₂), effectively decoupling O₂ generation from cellular needs (e.g. water), and instead to a waste product of cellular respiration as shown in FIG. 1. In this “inverse-breathing” system, CO₂ released from respiring cells in the host tissue (Equation 1) is recycled into O₂ by lithium peroxide (Li₂O₂), thereby comprising a self-sustaining system (Equation 2; FIG. 1A).

Glucose+6 O₂→6 CO₂+6 H₂O+ATP Equation 1

2Li₂O₂₊₂CO₂→2 Li₂CO₃+O₂ Equation 2

In human tissues, CO₂ is ubiquitously available at a partial pressure (pCO₂) regulated to approximately 40 mmHg (Tannock, “Oxygen Diffusion and the Distribution of Cellular Radiosensitivity in Tumours,” Br. J. Radiol. 45:515-524 (1972), which is hereby incorporated by reference in its entirety). Additionally, when exposed to high glucose levels such as after a meal, β cell CO₂ production and O₂ demand are increased in tandem (Cole and Logothetopoulos, “Glucose Oxidation (14-CO2 Production) and Insulin Secretion by Pancreatic Islets Isolated from Hyperglycemic and Normoglycemic Rats,” Diabetes 23:469-473 (1974), which is hereby incorporated by reference in its entirety). Accordingly, CO₂ is both a ubiquitous and dynamic reactant optimally suited to control O₂ delivery to encapsulated cells. As demonstrated herein, by immersing Li₂O₂ in a perfluorocarbon (PFC) oil, which has the capacity to dissolve high concentrations of CO₂ and O₂, and isolating the formulation from the hydrogel-encapsulated cells through a gas-permeable, liquid-impermeable silicone membrane, the self-regulated release of O₂ is achieved without influencing the aqueous cellular environment. Given the high O₂ content of Li₂O₂ (2.1-fold higher than CaO₂ considering commercial purities), and the fact that the O₂ generation and cellular encapsulation compartments are separated, O₂ release can last for months with one implantation and may be further extended by increasing the loading capacity or through refilling.

Described herein are inverse-breathing devices, referred to as “Inverse Breathing Encapsulation Device (iBED)” that produce O₂ in a CO₂-responsive and sustainable manner, mitigating hypoxia in proximally encapsulated cells during incubation in a low O₂ environment. A first-generation iBED prototype showed that its implementation significantly improved survival and function of rat islets in immunocompetent, streptozotocin (STZ)-induced diabetic mice within the poorly oxygenated subcutaneous site. A computational model, validated by in vitro O₂ measurements and pO₂ distribution mapping, guided the optimization of the iBED system, yielding a device which achieved diabetes reversal for over 3 months in the subcutaneous rat-to-mouse model. These optimized designs featured a terminal tank (i.e., reservoir device) containing the PFC-immersed Li₂O₂ formulation connected to a hollow gas permeable tube coated with a cell encapsulation hydrogel layer. Finally, a scaled-up device was developed for large animal testing. Surprisingly, rat islet survival was observed after retrieval at one and two months in a subcutaneous rat-to-pig xenotransplantation, despite the wide species gap and challenging subcutaneous environment. The cell encapsulation devices and systems described herein can overcome several outstanding challenges in oxygenating encapsulated cells and represents considerable progress in the use of translatable long-term O₂-supplementing technologies for cell replacement therapies.

### BRIEF DESCRIPTION OF THE DRAWINGS

FIGS. 1A-1G show an inverse-breathing system for encapsulated cells. (FIG. 1A) A schematic representation of the inverse-breathing system: islets were encapsulated in a semipermeable alginate hydrogel which permitted the transport of nutrients and the delivery of insulin but prevented the infiltration of immune effector cells; CO₂ released from the cells was rapidly transported in the gas phase to PFC-encapsulated Li₂O₂ particulates, contained within a gas-permeable, liquid-impermeable silicone membrane, whereby CO₂ was recycled into O₂. (FIG. 1B) Fabrication steps for a test construct. (FIG. 1C) A schematic representing the CO₂-responsive O₂ generation confirmation test: a construct was submerged in Na₂SO₃-deoxygenating buffer and aerated with CO₂; the CO₂ diffused through the silicone tubing and reacted with the encapsulated Li₂O₂, leading to O₂ bubble generation and accumulation on the construct's surface; the O₂ bubbles attached to the bottom of the O₂ probe, diffused through the gas permeable membrane of the O₂ probe, and were detected by the electrode. (FIG. 1D) A digital image of a test construct. (FIGS. 1E-1F) Digital images of the test assembly during N₂ aeration (FIG. 1E) and CO₂ aeration (FIG. 1F) (arrows indicate generated O₂ bubbles). (FIG. 1G) O₂ production over time measured by the submerged O₂ probe near the device (arrows indicate aeration phase start times).

FIGS. 2A-2H show subcutaneous (SC) xenotransplantation in minipigs. (FIGS. 2A and 2B) Device assembly of iBEDv3S. (FIG. 2C) Digital and stereo microscope images showing O₂ bubble formation along the silicone scaffold after incubation in 5% CO₂, 20% O₂. (FIG. 2D) O₂ measurement setup (left) showing the O₂ probe inserted into a bubble (the red arrow indicates the O₂ microsensor tip) and O₂ readings (right) in bubbles and buffer. (FIG. 2E) Stereo microscope images of the iBEDv3S device with the rat islet encapsulation hydrogel. (FIG. 2F) Surgical transplantation procedure in a Gottingen minipig: (1) two incisions were made and connected by a pipet through the deep SC space as a guide wire; (2) a hollow silicone tube was fed along the pipet through the incisions, and the pipet was withdrawn; (3) the device was inserted into the hollow silicone tube from the cell encapsulation end; (4) the tank was situated at the end of the semicircular incision in the deep SC pocket and (5) the hollow silicone tube was pulled out through the other incision, leaving the device in the SC space; (6) both incisions were suture-closed. (FIG. 2G) H&E staining of the retrieved device at 1 month; inset: a stereo microscope image of a region of hydrogel. (FIG. 2H) Immunohistochemical staining of retrieved islets.

FIGS. 3A-3B show maintained CO₂-responsive O₂ production in an iBEDv3 retrieved from a mouse at 3 months. (FIG. 3A) An iBEDv3 device, retrieved from mice at 92 d, was truncated at the PDMS adapter and incubated in 5% CO₂, 20% O₂. O₂ content was measured in the bubbles forming at the PDMS adapter-buffer interface (the twisted silicone tubing of iBEDv3 was cut), and in the bulk buffer, indicating maintained CO₂-responsive O₂ production at this time point. Arrows indicate the O₂ microsensor tip. (FIG. 3B) Similar measurements performed on a retrieved control device showed near the equilibrium level (˜0.25 mM, 20% O₂) in measurements of the probe near the tank port and in the buffer. Arrows indicate the O₂ microsensor tip.

FIGS. 4A-4E show pO₂ measurements in the lumen of iBEDv2 and the corresponding control device. (FIG. 4A) Stereo microscope image of the iBEDv2 adapted for lumen pO₂ measurement. (FIG. 4B) Schematic representation of the O₂ needle sensor used for measurement. (FIG. 4C) Digital image of the measurement test: the device was submerged in a buffer equilibrated to 5 CO₂, 5% O₂; the O₂ microsensor was then inserted through the PDMS attachment and into the (gas phase) lumen of the silicone tubing to obtain a measurement (red arrow indicates microsensor tip inserted in the measurement adapter); the test was performed in a closed gas chamber (FIG. 4D) supplied with a continuous gas (5% CO₂, 5% O₂) flow. (FIG. 4E) Measured lumen pO₂ in iBEDv2 (n=2) and control devices (n=3) (**p<0.01). We note that the device was immerged saline in a 50 mL Falcon tube and incubated in a hypoxia incubator with 5% CO₂, 5% O₂ for two weeks before the measurements. Measurements were taken at the end of this two-week period. The Falcon tube was sealed before being taken out from the hypoxia incubator and transferred into a sealed and pre-equilibrated hypoxia chamber to conduct the oxygen measurement. The closed hypoxia chamber was supplied with a continuous gas flow (5% CO₂, 5% O₂, 90% N₂) from a gas cylinder. Then, the oxygen probe was introduced into the chamber from a top window and sealed with a PDMS washer. After checking that the oxygen condition was equilibrated to 5% O₂ inside the chamber, the oxygen probe was insert into the device lumen for the final measurement.

FIGS. 5A-5D show the salt leaching modification of the gas permeable membrane surface for acceptance of the hydrogel layer. (FIG. 5A) Schematic representing a two-part hollow mold for silicone tubing surface roughness modification. (FIG. 5B) Schematic showing the gap (red arrows) between the tubing and hollow mold used to control the coating thickness, and a solid rod (yellow) used to keep the tubing straight inside the mold. (FIG. 5C) Digital and (FIG. 5D) stereo microscope images of the NaCl/PDMS modified surface before leaching.

FIGS. 6A-6B show iBEDv1 components. (FIG. 6A) Digital image showing 3D-printed terminal tank before (left) and after (right) coating with additional resin. (FIG. 6B) Digital image showing the silicone tubing (surface unmodified) attached to the Li₂O₂/PFC-filled terminal tank via a PDMS adapter.

FIGS. 7A-7R show the iBEDv3 design and 3-month diabetes correction in mice. (FIG. 7A-7C) Assembly of the twisted silicone tubing and PDMS adapter in iBEDv3. (FIG. 7D-7E) Liquid perfusion test of the twisted silicone. (FIG. 7F) Stereo microscope image of an assembled iBEDv3. (FIG. 7G) Gas permeability test of the silicone tubing. (FIG. 7H) Schematic and (FIG. 7I) stereo microscope image showing the complete iBEDv3 device with the rat islet encapsulation hydrogel. (FIG. 7J) BG measurements in C57BL6/J mice following SC transplantation of iBEDv3 devices (blue and pink; n=10), and control devices (red; n=7) over 92 d; ***p<0.001 (control versus all iBEDv3). (FIG. 7K) IPGTT test at 90 d mean±SD, ****p<0.0001 (iBEDv3 versus diabetic mice, iBEDv3 versus control device, and healthy mice versus control device), ***p<0.001 (iBEDv3 versus healthy mice), n.s. (p>0.05; control device versus diabetic mice). (FIG. 7L) Static GSIS test of retrieved iBEDv3 devices (n=3); mean SD, **p<0.01. (FIG. 7M) 3D reconstruction (left) and transverse 2D cross-section (right) of micro-CT scan images of an iBEDv3 device in a mouse. (FIG. 7N) Stereo microscope images of one iBEDv3 device retrieved at 92 d. (FIG. 7O-7P) CO₂ responsiveness of a retrieved iBEDv3 device incubated under 5% CO₂ with (FIG. 7O) the cell encapsulation hydrogel removed and (FIG. 7P) the twisted silicone tubing cut (red arrows indicate O₂ bubble formation). (FIG. 7Q) H&E and (FIG. 7R) immunohistochemical staining of retrieved iBEDv3 devices.

FIGS. 8A-8C show viability of encapsulated cells under hypoxic incubation. (FIG. 8A-8B) Fluorescent microscopy images of INS-1 cells stained for viability (alive=green, dead=red) following 24-h culture in 1% O₂, 5% CO₂ in (FIG. 8A) Li₂CO₃/PFC-containing control device and (FIG. 8B) Li₂O₂/PFC-containing device (iBED). (FIG. 8C) Quantification of the fluorescence intensity of the live-staining and dead-staining cells in the selected regions (blue boxes) of the control device and the iBED. Note, the silicone tubing in the device is indicated by the white dashed lines. The iBED yielded a 2.7-fold improvement in cell viability in comparison to the control device (two-way ANOVA followed by Tukey's post-hoc p-value adjustment, ****p<0.0001), quantitatively confirming the ability of iBED to improve cell survival in hypoxic conditions.

FIGS. 9A-9D show computational model development. (FIG. 9A) Schematics illustrating components of the control, iBEDv1, and iBEDv2 designs (left column) and iBEDv3 design (right column) including the alginate, silicone tubing, tubing lumen (air for control, iBEDv2, iBEDv3 designs, PFC for iBEDv1 design), and randomly distributed islets. (FIG. 9B) A high magnification image showing the randomly distributed islets in the alginate. (FIG. 9C) An image showing the nonuniform mesh on the alginate surface (left) and on individual simulated islets (right). (FIG. 9D) An annotated schematic showing the dimensions for each design.

FIG. 10 shows model boundary conditions. Schematic representation of model-implemented boundary conditions in the (top to bottom) control device, iBEDv1, iBEDv2, and iBEDv3. In all designs, boundary pO₂ was a constant of 40 mmHg along the length of the alginate cylinder and at the face representing the device end, the combination of which represented the device-host interface. In the control design, a no-flux condition (blue) was implemented on all surfaces representing the tank-cylinder interface. In iBEDv1, iBEDv2, and iBEDv3 designs, O₂ generation from the tank was implemented as a constant surface flux (red) on the surface of the tubing lumen(s). No flux conditions were implemented on the tank-silicone and tank-alginate interfaces on this face in all designs. Concentration of oxygen (c_{O}_{₂}) was converted into pO₂ by the relationship shown in the boxed equation.

FIGS. 11A-11G show computational model-guided design optimization. (FIG. 11A) Schematics representing design iterations. (FIG. 11B) Schematics representing simulated geometry for each design, showing the silicone tubing structures (top), and full geometry (bottom) including the randomly seeded islets (yellow), alginate (green), silicone tubing (grey), and lumen material (white=air, yellow=PFC). (FIG. 11C) pO₂ distributions in three cross sections (indicated by the diagram on the left) of each design showing low and uniform pO₂ in the control, a gradient of pO₂ in the iBEDv1, and high and uniform pO₂ in iBEDv2 and iBEDv3. A high magnification image selected from one cross section is also presented (top); white regions represent necrosis. (FIG. 11D) Islet surface pO₂ distributions from the xy- (top) and yz- (bottom) perspectives. (FIG. 11E) Scatter plots of islet volume-average pO₂ versus islet diameter (black circle indicates volume-weighted average islet diameter and pO₂; each dot represents one islet). (FIG. 11F) Frequency of islet volume-average pO₂ in each design, showing high concentration of poorly oxygenated islets in the control and iBEDv1, and a high concentration of well-oxygenated islets in iBEDv2 and iBEDv3; solid lines represent medians, dashed lines represent first and third quartiles. (FIG. 11G) pO₂ along a center line of the device.

FIGS. 12A-12D show simulation-predicted islet oxygenation. (FIG. 12A-12D) Islet survival and oxygenation in (top to bottom) the control device, iBEDv1, iBEDv2, and iBEDv3 from one iteration selected at random; each point represents a simulated islet. Scatter plots of islet diameter versus predicted individual islet volume fraction of (FIG. 12A) metabolically unresponsive (pO₂<8 mmHg) tissue and (FIG. 12B) necrotic (pO₂<0.08 mmHg) tissue. (FIG. 12C) Predicted net volume fraction of necrotic tissue (red), metabolically unresponsive tissue (yellow) and functional tissue (pO₂>8 mmHg). (FIG. 12D) Scatter plots of islet diameter versus predicted oxygenation according to z position (left column) and radial position (right column) from a single iteration.

FIGS. 13A-13L show improvement of cell survival during in vitro hypoxic incubation and in vivo. (FIG. 13A) Fabrication of a Li₂O₂/PFC@silicone construct. (FIGS. 13B-13C) Fluorescent microscopy images of INS-1 cells stained for viability (alive=green, dead=red) following 24-h culture in 1% O₂, 5% CO₂ in Li₂CO₃/PFC controls (FIG. 13B) and Li₂O₂/PFC-containing constructs (FIG. 13C). (FIGS. 13D-13E) Immunostaining of nuclei (DAPI; blue) and pimonidazole-protein adducts (green) following 24-h culture in 1% O₂, 5% CO₂ of islets in (FIG. 13D) Li₂CO₃/PFC controls and (FIG. 13E) Li₂O₂/PFC-containing constructs. (FIGS. 13F-13G) H&E stained islets (left) and insulin- (red) and glucagon- (green) immunostained islets (right) after 24 h hypoxic culture in (FIG. 13F) Li₂CO₃/PFC controls (yellow arrow indicates nuclear fragmentation; yellow outline circumscribes pyknotic cells) and (FIG. 13G) Li₂O₂/PFC-containing constructs. (FIG. 13H) Stereo microscope image of the surface-modified silicone tubing. (FIG. 13I) Microscope image of construct with encapsulated rat islets. (FIG. 13J) BG readings in Li₂O₂/PFC-containing islet encapsulation constructs (n=3) and Li₂CO₃/PFC islet encapsulation controls (n=3) over a 4-week transplantation period; ***p<0.001. (FIG. 13K) H&E and (FIG. 13L) nuclei (DAPI=blue) and insulin (red) stained islets retrieved from Li₂O₂/PFC-containing construct.

FIGS. 14A-14K show iBEDv1 design and diabetes reversal in mice following SC transplantation. (FIG. 14A) Device assembly of iBEDv1. (FIG. 14B) Digital image of the 3D printed terminal tank (left) and an annotated schematic (right) showing the direction of gas transfer through the PDMS adapter. (FIG. 14C) Digital image of the iBEDv1 device. (FIG. 14D) Microscope image of the iBEDv1 device, including the rat islet encapsulation hydrogel. (FIG. 14E) BG measurements in C57BL6/J mice following SC transplantation of control devices (n=5) and iBEDv1 devices (n=10) over 60 d; ***p<0.001 (control versus iBEDv1). (FIG. 14F) IPGTT test at 58 d; mean SD, ****p<0.0001 (all treatment comparisons). (FIG. 14G) Digital image of a retrieved device (stripped of its hydrogel layer) in 5% CO₂ buffer, showing bubble formation at the scaffold surface. (FIG. 14H) H&E stained slides after retrieval emphasizing the fibrotic layer adjacent to the device. (FIG. 14I) Nuclei (DAPI; blue) and α-SMA (red) immunostaining showing the fibrotic layer adjacent to the device surface (the asterix indicates device). (FIG. 14J) H&E and (FIG. 14K) nuclei (DAPI; blue), insulin (red), and glucagon (green) immunostaining in islets retrieved from iBEDv1 device.

FIG. 15 shows Clark-type O₂ sensor. Schematic illustrating the components of a Clark-type O₂ sensor (Vernier). O₂ diffuses through a gas permeable membrane to an O₂-reducing cathode, which is polarized against an internal Ag/AgCl anode. pO₂ is linearly proportional to the detected current strength.

FIG. 16 shows a graph of no CO₂ responsiveness in the control Li₂CO₃/PFC@silicone tubing. O₂ concentration recordings of the sensor (Vernier) submerged near the control sample during N₂ and CO₂ aeration.

FIGS. 17A-17B show the pre-experiment of pimonidazole as a hypoxia marker in INS-1 cells. Pimonidazole-protein adducts (green) and nuclei (DAPI; blue) staining of INS-1 cells (50 k cells per well in a 48-well plate) incubated in (FIG. 17A) a normal incubator (˜20% 02, 5% CO₂) and (FIG. 17B) a hypoxia incubator (1% O₂, 5% CO₂) for 24 h.

FIGS. 18A-18B show staining of islet hypoxia. Microscope images of staining of nuclei (DAPI; blue) and pimonidazole-protein adducts (green) following 24-h culture in 1% O₂, 5% CO₂ of islets in (FIG. 18A) a Li₂CO₃/PFC@silicone control and (FIG. 18B) a Li₂O₂/PFC@silicone construct. (Images in FIG. 18A are separated channels from the merged image in FIG. 13D, and images in FIG. 18B are separated channels from the merged image in FIG. 13E.)

FIGS. 19A-19M show morphology and immunohistochemistry of islets after hypoxic incubation. Rat islets encapsulated in (FIGS. 19A-19F) Li₂CO₃/PFC@silicone controls and (FIGS. 19G-19L) Li₂O₂/PFC@silicone constructs (iBED) incubated at 1% O₂, 5% CO₂ for 24 hours. (FIGS. 19A-19C) H&E staining of islets in control samples showed a corona of healthy cells, whereas cells in the islet core exhibited pyknosis (cells circumscribed in the dashed yellow line) and nuclear fragmentation (yellow arrows). (FIGS. 19D-19F) Nuclei (DAPI; blue)/insulin (red)/glucagon (green) immunostaining was positive for insulin and glucagon in the islet periphery but negative in the islet core. (FIGS. 19G-19I) H&E staining of islets in Li₂O₂/PFC@silicone samples showed round morphologies no indicators of hypoxia. (FIGS. 19J-19L) Nuclei (DAPI; blue)/insulin (red)/glucagon (green) immunostaining showed islets were intact and positive for insulin throughout and glucagon in peripheral cells, suggesting maintained function. (FIG. 19M) Comparison of islet function in the control device and the iBED by quantification of insulin and DAPI fluorescence intensity of the stained islets to evaluate the islet functionality and pyknosis (shrunken and dark nuclei)/karyorrhexis (fragmented nuclei) in islets, respectively. Specially, islets in the control devices showed a 1.86-fold reduction (two-way ANOVA followed by Tukey's post-hoc p-value adjustment, **p<0.01) of insulin expression due to the non-insulin secretion cells in the hypoxic core and 1.25-fold reduction in DAPI content (two-way ANOVA followed by Tukey's post-hoc p-value adjustment, n.s., p>0.05) because of the shrunken or fragmented nuclei (though this was not found to be statistically significant) compared to islets in the iBED devices.

FIGS. 20A-20D show Li₂O₂/PFC@silicone construct retrieved from SC rat-to-mouse transplantation. H&E staining of rat islet encapsulated Li₂O₂/PFC@silicone construct retrieved from mice at 4 weeks. (FIGS. 20A-20D) Peripheral high magnification images show both healthy islets and unhealthy islets (red arrows).

FIGS. 21A-21C show SC implantation of iBEDv1 in mice. Digital images showing a mouse with a dorsolateral SC transplanted iBEDv1 directly after surgery (FIGS. 21A and 21B) and two weeks post-surgery (FIG. 21C).

FIGS. 22A-22C show biocompatibility of the iBEDv1 and the terminal tank. (FIG. 22A) pH monitoring of saline solution cultured with iBEDv1 devices over 10 days. Normal untreated saline solution was used as the control. No significant pH change was observed, and no statistically significant difference compared with the control (one-way ANCOVA, n.s., p>0.05). (FIG. 22B) Digital image showing the dorsolateral SC transplantation of iBEDv1 in mice emphasizing the terminal tank. (FIG. 22C) Masson's trichrome images of the tissue surrounding the terminal tank following device retrieval at 2 months, showing no indications of an adverse reaction.

FIGS. 23A-23C show maintained CO₂ responsiveness and islet morphology in a retrieved iBEDv1. (FIG. 23A-23B) Digital images at two time points of a rat islet encapsulating iBEDv1 retrieved at 2 months from a mouse, incubated at 5% CO₂, 5% O₂ (note, the cell encapsulation hydrogel was stripped prior to incubation). Bubbles, indicating active CO₂-responsive O₂ production, formed (FIG. 23A) near the terminal tank first, and (FIG. 23B) eventually along the tubing lumen, suggesting a z-gradient of O₂. (FIG. 23C) Microscope images of the encapsulated islets, showing round and well-defined islet morphology in regions near the tank.

FIGS. 24A-24D show islet health in a retrieved iBEDv1 at 2 months from a mouse with well-controlled BG. Microscope images of H&E staining (FIG. 24A) and nuclei (DAPI; blue)/CD3 (T cell marker; red) staining (FIG. 24B) of the device-host boundary show no signs of inflammation and rare, sporadic CD3+ cells (T cells). H&E staining (FIG. 24C) and nuclei (DAPI; blue)/insulin (red) immunostaining (FIG. 24D) of islets in regions far away from the tank indicating a few partially unhealthy cells.

FIGS. 25A-25E show islet health in a retrieved iBEDv1 at 2 months from a mouse with poorly controlled BG. (FIG. 25A) H&E staining of the fibrotic tissue surrounding the implanted iBEDv1 showing a vigorous inflammatory response, a layer of dense immune cells located at the device-host boundary. (FIG. 25B) nuclei (DAPI; blue)/CD3 (T cell marker; red)/CD68 (macrophage marker; green) staining showing mass accumulation of CD3 positive T cells at the boundary (asterisk indicates the device side of the device-host boundary) and moderate macrophage accumulation in fibrotic tissue several cell layers away from the boundary. (FIG. 25C) DAPI/CD68/α-SMA (myofibroblasts, red) staining, showing myofibroblasts and macrophages in fibrotic tissue several cell layers away from the boundary, few myofibroblasts and macrophage near the interface. (FIG. 25D) H&E staining and (FIG. 25E) DAPI/insulin (red) staining of a select islet, showing functional core cells, but unhealthy and non-functional peripheral cells.

FIGS. 26A-26B show islet health in a retrieved control device (i.e. a device similar to iBEDv1 but without the inverse-breathing feature) at 2 months from a mouse. Microscope images of (FIG. 26A) H&E staining of a retrieved control sample showing several necrosed islets with severe karyorrhexis or complete loss of nuclei. (FIG. 26B) Nuclei (DAPI; blue)/insulin (red) immunostaining showing weak positive signals for both markers; no DAPI signal was observed in the necrotic core, and only few insulin positive cells were detected in peripheries.

FIGS. 27A-27H show EPR O₂ imaging. (FIG. 27A) A digital image of the JIVA-25 instrument (red arrow indicates sample location). Inset figure shows a digital image of an iBEDv3 submerged in OX063-d24 aqueous buffer (blue arrow indicates the iBEDv3). (FIG. 27B) Schematic representation of the EPR instrument setup. (FIG. 27C) Chemical structure of OX063-d24. (FIG. 27D) Calibration curve used for obtaining pO₂ from relaxation rates of OX063-d24. (FIGS. 27E-27H) Average pO₂ measurements in the whole system (solution and device) versus time and cross-section pO₂ distributions on two planes (side view and top view) at three times (indicated by the arrows in the time series plots). The asterisks indicate the tank located at the bottom of the glass tube, and the hollow empty regions on top view images indicate the silicone tubing of the iBEDv3. (FIG. 27E) A control device sample and (FIG. 27F) an iBEDv3 sample in aqueous buffer subjected to 5% CO₂, 5% O₂, 90% N₂ (pink dashed line indicates equilibrium O₂:pO₂=40 mmHg, i.e. 5% O₂). (FIG. 27G) A control device sample and (FIG. 27H) an iBEDv3 device sample in 1% gelatin subjected to O₂-free 5% CO₂, 95% N₂. The red arrows indicate the regions of high pO₂ around the silicone tubing of the iBEDv3.

FIGS. 28A-28E show EPR O₂ imaging. (FIG. 28A) Schematic showing the container for the pO₂ distribution mapping study: the device was placed in spin probe solution within a sealed glass vial; a gas inlet was used for sample aeration, and a gas outlet was included for venting. (FIG. 28B) An abbreviated aeration protocol used in the EPR O₂ imaging studies. Average pO₂ measurements in the whole system (i.e. solution and device) versus time and cross-section pO₂ distributions on two planes (side view and top view) at four times (indicated by the arrows in the time series plots) in an iBEDv3 in 1% gelatin subjected to a gas mixture of (FIG. 28C) 5% CO₂, 95% N₂ and (FIG. 28D) 5% CO₂, 5% O₂, 90% N₂. (FIG. 28E) Cross-section pO₂ distribution in 1% gelatin (no device) subjected to overhead flow with 5% CO₂, 5% O₂, 90% N₂ (no bubbling phase after deoxygenation), showing poor penetration of O₂ over time.

FIGS. 29A-29H show design improvements in iBEDv3. (FIG. 29A) Schematic of the iBEDv1 device, highlighting the PDMS adapter connecting the terminal tank to the silicone tubing. This required slow CO₂ and O₂ transport in the solid phase (through the PDMS adapter) and the liquid phase (through the PFC in the lumen). (FIG. 29B) Digital image of an iBEDv1 retrieved at 2 months from a mouse, showing tank inflation due to positive pressure from gas build-up in the terminal tank. (FIG. 29C) Digital image of the retrieved iBEDv1 following incubation in 5% CO₂, 5% O₂, showing bubble formation only near the terminal tank (red arrows indicate bubbles). (FIG. 29D) Schematic of the iBEDv3, highlighting the PDMS adapter penetrated by the twisted hollow silicone tubing, allowing rapid CO₂ and O₂ transfer in the gas phase. (FIG. 29E) Digital image of a retrieved iBEDv3 at 3 months from a mouse, showing the absence of tank inflation. (FIG. 29F) Digital image of the iBEDv3 device following incubation in 5% CO₂, 5% O₂, showing consistent bubble formation both near and far from the terminal tank (red arrows indicate bubbles). (FIGS. 29G-29H) Comparison of therapeutic function of iBEDv1 and iBEDv3 in mice. (FIG. 29G) Engraftment percent of the iBEDv1 and iBEDv3 summarized from the pulled BG data of in vivo studies (FIG. 14E and FIG. 7J) (Mantel-Cox, *p<0.05). Device “failure” was defined as 3 consecutive readings over 250 mg/dL. (FIG. 29H) IPGTT data pulled from the iBEDv1 and iBEDv3 in vivo studies (FIG. 14F and FIG. 7K) (two-way ANOVA, ***p<0.001).

FIGS. 30A-30F shows micro-CT scans of the iBEDv3 device implanted in a mouse for 3 months. (FIGS. 30A-30E) 3D reconstruction of micro-CT images of an iBEDv3 in a mouse. (FIGS. 30A-30B) Top and (FIGS. 30D-30E) side views of the implanted device in a mouse. An intensity threshold was applied to the images, isolating the lumens of the twisted silicone tubing, suggesting that a gas phase was maintained (note: four different colors, one for each lumen, were applied retroactively). The isolated lumens of the twisted tubing are shown in (FIG. 30C). (FIG. 30F) Two transverse 2D cross-sections of the iBEDv3 in a mouse, illustrating the preserved hollow structure of the silicone tubing both within the encapsulation domain (left) and the tank (right).

FIG. 31 shows islet health in an iBEDv3 and surrounding fibrotic tissue retrieved from a mouse at 3 months. H&E staining of a superficial section of alginate with surrounding fibrotic tissue in an iBEDv3. High magnification images (right panel) showing several healthy islets at locations proximate, intermediate, and distal to the tank.

FIGS. 32A-32D shows islet health in an iBEDv3 retrieved from a mouse at 3 months. H&E and nuclei (DAPI; blue)/insulin (red)/glucagon (green) immunostaining of a superficial section (FIGS. 32A-32B) and a deeper section (FIGS. 32C-32D) of alginate in an iBEDv3. Numerous healthy and insulin/glucagon positive islets were found in both sections. Note: the fibrotic capsule was removed during retrieval.

FIG. 33 islet health in a control device (i.e. a device similar to iBEDv3 but without the inverse-breathing feature) retrieved from a mouse at 3 months. H&E staining of a superficial section of a retrieved control iBEDv3 sample. Most observed islets were necrosed.

FIGS. 34A-34D shows microsensor O₂ measurements in an iBEDv3S for pig transplantation. (FIG. 34A) Schematic showing the components of the O₂ microsensor (OX-100, Unisense) used for direct measurement. O₂ measurements in (FIG. 34B) a bubble proximate to the terminal tank, (FIG. 34C) the buffer, and (FIG. 34D) a bubble distal to the terminal tank following incubation in 5% CO₂, 20% O₂. Arrows indicate the microsensor tip.

FIGS. 35A-35J viability of rat islets from a control device (i.e. a device similar to iBEDv3S but without the inverse-breathing feature) retrieved at 1 month following SC implantation in a Gottingen minipig. (FIG. 35A) Stereo microscope image of a region of hydrogel from the retrieved sample; islets were all dark, with a rough surface. (FIG. 35B) H&E staining of the collected hydrogel from the retrieved device. No healthy islets were found in the whole section. (FIGS. 35C-35D) H&E staining and (FIGS. 35E-35F) nuclei (DAPI; blue)/insulin (red) immunostaining showing some necrosed islets with severe karyorrhexis; fragmented nuclei and sporadic insulin positive cells were detected. (FIGS. 35G-35H) H&E staining and (FIGS. 35I-35J) nuclei/insulin immunostaining showing some necrosed islets with complete loss of nuclei, faint DAPI signal in the islets remains.

FIGS. 36A-36I shows H&E staining of one rat islet encapsulation iBEDv3S retrieved at 1 month following SC implantation in a Gottingen minipig. (FIGS. 36A-36H) Numerous healthy islets were observed in the retrieved device (FIG. 2G), though some cells in islet peripheries were found to be apoptotic in a few islets (arrows in FIG. 36D and FIG. 36H), likely due to xenogeneic immunological response rather than hypoxia, which usually causes necrosis in the islet center. (FIG. 36I) Comparison of islet function in this iBEDv3S (FIGS. 36E-36H) and the control device in above FIGS. 35E-35F and FIGS. 35I-35J by quantification of insulin and DAPI fluorescence intensity of the stained islets to evaluate the islet functionality and karyorrhexis (fragmented nuclei)/loss of nuclei in islets, respectively. Specially, insulin expression was 9.6-fold higher (two-way ANOVA followed by Tukey's post-hoc p-value adjustment, ****p<0.0001) and DAPI content was 3.4-fold higher (two-way ANOVA followed by Tukey's post-hoc p-value adjustment, **p<0.01) in islets from iBEDv3S compared to islets from control device, quantitatively confirming that the iBEDv3S yielded substantial improvements in islet survival and function in pigs.

FIGS. 37A-37H shows islet viability in one rat islet encapsulation iBEDv3S retrieved at 2 months following SC implantation in a Gottingen minipig. (FIG. 37A) Stereo microscope image and (FIG. 37B) bright-field microscope images showing a region of hydrogel from the retrieved sample; some healthy islets appeared as yellow and maintained a smooth and intact morphology, and some unhealthy islets showed as dark, and presented a rough surface and loose morphology. (FIG. 37C) H&E staining and (FIG. 37D) nuclei (DAPI; blue)/insulin (red) immunostaining of some islets with partially unhealthy cells. (FIG. 37E) Stereo microscope image and (FIG. 37F) bright-field microscope images showing a region of hydrogel from the retrieved sample; most islets were healthy, appearing as yellow with maintained smooth and intact morphology. (FIG. 37G) H&E staining and (FIG. 37H) nuclei/insulin immunostaining of select healthy islets.

FIGS. 38A-38B shows prospective designs extending the lifetime of O₂ supply indefinitely. Schematics showing (FIG. 38A) a potential modular design for a tank that can be replaced and (FIG. 38B) a design which features a portal in the terminal tank through which the expired Li₂O₂/PFC can be withdrawn and refilled.

FIGS. 39A-39E shows prospective designs to increase capacity. (FIGS. 39A-39C) Schematics showing designs which increase the volume of transplantable islets including (FIG. 39A) extending length of the cell encapsulation tubing, (FIG. 39B) implementing multiple parallel cell encapsulation domains connected to a single tank, and (FIG. 39C) expanding the diameter of the inner of the tank. (FIG. 39D) Digital images (left, middle) and a schematic showing a design for a planar device prototype with multiple aeration channels connected to the terminal tank to increase islet capacity. (FIG. 39E) A schematic showing a design to improve the O₂ delivery rate to support higher islet densities, by introduction of a silicone balloon in the terminal tank.

### DETAILED DESCRIPTION OF THE INVENTION

The present invention relates to devices, systems, and components thereof suitable for oxygenating encapsulated cells. A first aspect of the present invention relates to a reservoir device for providing oxygen to encapsulated cells. This reservoir device includes a reservoir suitable for enclosing a liquid. The device further includes a liquid contained within the reservoir, where the liquid is a permeable to gas, and an oxygen-generating compound, where the oxygen-generating compound is immersed in the liquid contained within the reservoir.

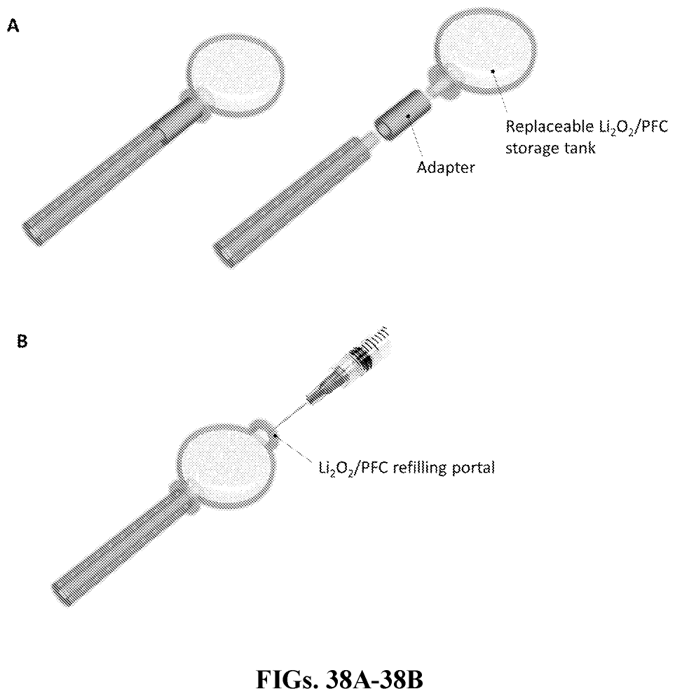

In one embodiment, the reservoir further comprises an opening. In another embodiment, the reservoir comprises a plurality of opening. The one or more openings of the reservoir device each optionally comprise a fitting. The fitting is suitable for coupling the reservoir device, via one or more of the openings, to a cell encapsulation device (see FIG. 38A), e.g., a cell encapsulation device as described herein or other cell encapsulation devices known in the art (see e.g., U.S. Pat. No. 10,493,107 to Ma et al., and WO2021/202945 to Ma et al., which are hereby incorporated by reference in their entirety. Suitable fittings should allow for or facilitate easy connection and disconnection of a reservoir device to a cell encapsulation device. This will allow for the reservoir device that is coupled to an implantable cell encapsulation device to be removed and replaced in vivo.

In any embodiment, the cell encapsulation device is connected to the reservoir device via a gas impermeable hollow tubing or other adaptor, with suitable fittings allowing gas permeation (either in the gas phase or through a gas-permeable material) through both ends. Suitable gas impermeable materials include polymer material such as polystyrene, polyethylene and polycarbonate as well as glass and metals such as gold.

In any embodiment, the cell encapsulation device is connected to the reservoir device via a gas permeable or semi-gas permeable hollow tubing or other adaptor, with suitable fittings allowing gas permeation (either in the gas phase or through a gas-permeable material) through both ends. Suitable gas permeable materials are described below.

The one or more openings of the reservoir device are optionally capped with a gas permeable membrane. In one embodiment, the gas permeable membrane is a liquid impermeable membrane. In any embodiment, the gas permeable membrane is permeable to oxygen and carbon dioxide. Suitable gas permeable membrane materials are known in the art and include, for example, and without limitation, polystyrene, polyethylene, polycarbonate, polyolefin, ethylene vinyl acetate, polypropylene, polysulfone, polydimethylsiloxane (PDMS), polytetrafluoroethylene (PTFE) or compatible fluoropolymer, silicone or silicon copolymer, poly(styrene-butadiene-styrene), nylon, polycarbonate (PCTE), polyether ether ketone (PEEK), polyethersulfone (PES), polyester (PETE), polypropylene, polyvinylidene fluoride (PVDF) or combinations of these materials. Exemplary gas permeable membrane materials for use as a cap to the reservoir device and in other devices described herein include polydimethylsiloxane (PDMS), silicone, and polytetrafluoroethylene (PTFE), and combinations thereof.

The reservoir device as described herein comprises a reservoir, i.e., a receptacle, that is suitable for enclosing a liquid. In any embodiment, this reservoir comprises a wall that encloses the liquid. The wall of the reservoir is made of a biocompatible material and, in any embodiment, is impermeable to solids, liquids, and gases. Suitable reservoir wall materials are non-dissolvable, biocompatible, medical grade polymers, resins, and metals. Exemplary reservoir wall materials include, without limitation, a biocompatible resin, a medical grade alloy, titanium, titanium alloy, stainless steel, cobalt chrome alloy, nickel titanium alloy, gold, platinum, silver, iridium, tantalum, tungsten, and combinations thereof. Additional biocompatible materials that can be used alone or in combination with the aforementioned wall materials include anisotropic materials, polysulfone (PSF), nano-fiber mats, polyimide, tetrafluoroethylene/polytetrafluoroethylene (PTFE; also known as Teflon®), ePTFE (expanded polytetrafluoroethylene), polyacrylonitrile, polyethersulfone, acrylic resin, cellulose acetate, cellulose nitrate, polyamide, as well as hydroxylpropyl methyl cellulose (HPMC).

The reservoir encloses a gas permeable liquid. In any embodiment, the gas permeable liquid contained within the reservoir or the reservoir device is selected from perfluorocarbon (PFC) oil, mineral oil, silicone oil, and combinations thereof. Other suitable gas permeable liquids include, without limitation, perfluoropolyether (PFPE) synthetic lubricant, magnesium stearate, calcium stearate, zinc stearate, stearic acid, sodium stearyl fumarate, glyceryl di-behenate, hydrogenated vegetable oil, mineral oil, talc, and combinations thereof. The reservoir also comprises an oxygen-generating compound. In any embodiment, the oxygen-generating compound is immersed in the liquid contained within the reservoir. The oxygen-generating compound of the reservoir reacts with carbon dioxide to release oxygen. Suitable oxygen-generating compounds include, without limitation, lithium peroxide, sodium peroxide, potassium peroxide, potassium superoxide, calcium peroxide, magnesium peroxide, sodium percarbonate, and combinations thereof.

In any embodiment, the reservoir is configured to be refillable with oxygen-generating compound and/or the gas permeable liquid. In any embodiment, at least one of the one or more openings in the reservoir as described supra, are configured for coupling to a device suitable for providing replacement oxygen-generating compound and/or replacement gas permeable liquid (see e.g., FIG. 38B).

The reservoir device as described herein can take any shape, and the shape of the device may be dictated by its use in conjunction with a cell encapsulation device as described herein. For example, when the reservoir device is coupled to an implantable cell encapsulation device, the reservoir device may be designed to have a flattened configuration suitable for subcutaneous implantation. The area of implantation may also dictate a different configuration. Accordingly, the reservoir device as described herein may be produced in a flattened configuration, spherical, cylindrical configuration, a rectangular configuration or combinations thereof. In one embodiment, the device is configured for subcutaneous placement in a subject. In one embodiment, the device is configured for preperitoneal placement in a subject. In another embodiment, the device is configured for transperitoneal placement in a subject. In another embodiment, the device is configured for transcutaneous placement in a subject. In another embodiment, the device is configured for peritoneal placement in a subject. In another embodiment, the device is configured for intraperitoneal placement in a subject.

In any embodiment, the reservoir of the device has a diameter of about 10-80 mm. In any embodiment, the reservoir diameter is about 15-75 mm, about 20-70 mm, about 25-65 mm 30-60 mm, about 35-55 mm, about 40-50 mm, about 15-20 mm, about 15-25 mm, about 15-30 mm, about 15-35 mm, about 15-50 mm, about 50-55 mm, about 50-60 mm, about 50-65 mm, about 50-75 mm, or about 50-80 mm. In any embodiment, the reservoir of the device has a height of 3-30 mm. In any embodiment, the reservoir may have a height of about 5-30 mm, about 10-25 mm, about 15-20 mm, about 15-30 mm, about 20-30 mm, about 5-10 mm, or about 5-15 mm in height.

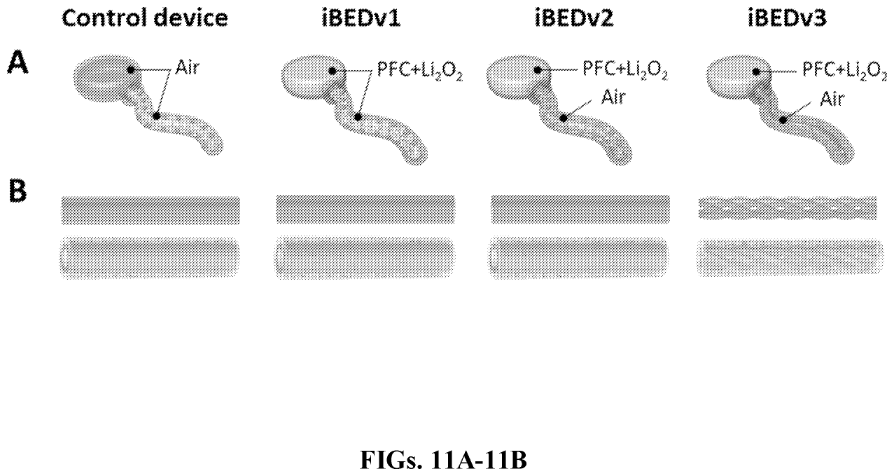

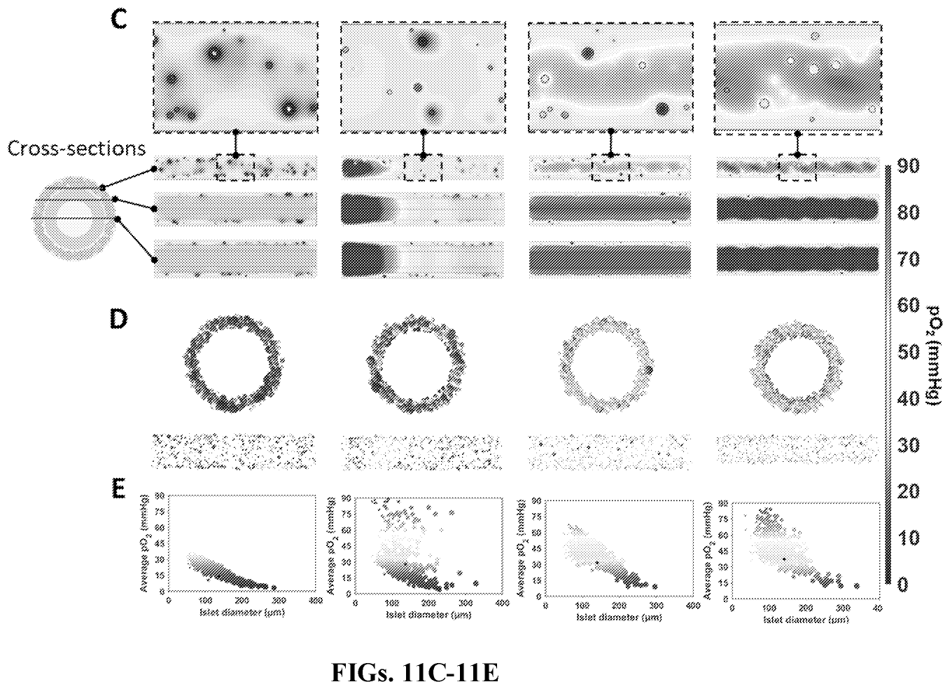

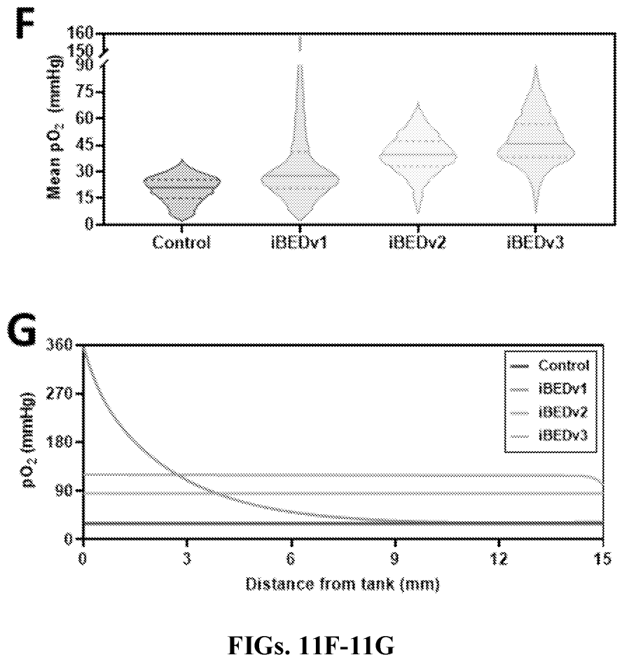

Another aspect of the present disclosure relates to cell encapsulation devices. The cell encapsulation devices described herein are configured to provide a source of oxygen to the encapsulated cells to enhance cell survival and function. In one embodiment, a cell encapsulation device of the present disclosure comprises a gas permeable membrane having proximal and distal ends. The gas permeable member encloses an inner space that extends longitudinally between the proximal and distal ends of the gas permeable membrane of the device. In one embodiment, the inner space is filled with an oxygen-generating compound (see e.g., FIG. 11A, iBEDv1). The cell encapsulation device further comprises a hydrogel layer that covers the outer surface of the gas permeable membrane. In one embodiment, the gas permeable membrane further comprises an opening at one of the proximal or distal ends. In any embodiment, the cell encapsulation device comprises two or more gas permeable membranes coupled together.

In another embodiment, the cell encapsulation device of the present disclosure comprises a gas permeable membrane having proximal and distal ends. The gas permeable member encloses an inner space that extends longitudinally between the proximal and distal ends of the gas permeable membrane of the device. In this embodiment, the inner space is at least partially filled with air (see e.g., FIG. 11A, iBEDv2). The cell encapsulation device further comprises a hydrogel layer that covers the outer surface of the gas permeable membrane. In one embodiment, the gas permeable membrane further comprises an opening at one of the proximal or distal ends. In any embodiment, the cell encapsulation device can comprise two or more gas permeable membranes coupled together.

In another embodiment, a cell encapsulation device of the present disclosure includes two or more elongated gas permeable membranes, where each of the gas permeable membranes has a proximal and distal end. The gas permeable membranes each enclose an inner space that extends longitudinally between the proximal and distal ends of the gas permeable membrane, where the inner space is at least partially filled with air. The proximal ends of the two or more elongated gas permeable membranes include an opening and the distal ends of the gas permeable are sealed. The two or more gas permeable membranes of the device are twisted helically about a longest axis shared by the membranes (see e.g., FIG. 11A-B, iBEDv3). The cell encapsulation device further comprises a hydrogel layer that covers the outer surfaces of the two or more helically twisted gas permeable membranes.

In accordance with this embodiment, the two or more elongated gas permeable membranes can be formed from two separate gas permeable membranes. Alternatively, the two or more gas permeable membranes can be formed from one gas permeable membrane that is folded in half to create two gas permeable membranes. In any embodiment, the proximal ends of the two or more elongated gas permeable membranes include an opening and the distal ends of the gas permeable are sealed. The distal ends of the gas permeable membranes may be sealed by a heat seal, a suture knot, a clamp, a rubber seal, or a screw closure.

The cell encapsulation device of this embodiment may comprise two helically twisted gas permeable membranes, three helically twisted gas permeable membranes, four helically twisted gas permeable membranes, five helically twisted gas permeable membranes, six helically twisted gas permeable membranes, seven helically twisted gas permeable membranes, eight helically twisted gas permeable membranes, nine helically twisted gas permeable membranes, ten helically twisted gas permeable membranes or more than ten helically twisted gas permeable membranes.

Suitable gas permeable materials for use in all of the cell encapsulation devices of the present disclosure are described supra, and include, for example and without limitation, polystyrene, polyethylene, polycarbonate, polyolefin, ethylene vinyl acetate, polypropylene, polysulfone, polydimethylsiloxane (PDMS), polytetrafluoroethylene (PTFE) or compatible fluoropolymer, silicone or silicon copolymer, poly(styrene-butadiene-styrene), nylon, polycarbonate (PCTE), polyether ether ketone (PEEK), polyethersulfone (PES), polyester (PETE), polypropylene, polyvinylidene fluoride (PVDF) or combinations of these materials. Exemplary gas permeable membrane materials for use as a cap to the reservoir device and in other devices described herein include polydimethylsiloxane (PDMS), silicone, and polytetrafluoroethylene (PTFE), and combinations thereof.

The gas permeable membranes of the cell encapsulation devices enclose an inner space that extends longitudinally between the proximal and distal ends of the membrane. In any embodiment, the gas permeable membrane forms a tube or tube-like structure to enclose the inner space. The tube or tube-like structure formed from the gas permeable membrane can have any geometrical cross-section, e.g., circular, triangular, square, rectangular, pentagonal hexagonal, heptagonal, octagonal, etc. In any embodiment, the tube is a cylindrical tube and has a tube diameter of about 0.5 mm to about 3 mm. In any embodiment, the cylindrical tube has a diameter of about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, and about 5 mm.

In any embodiment, the length of the gas permeable membranes, i.e., distance between proximal and distal ends, is about 1 centimeter to about 1 meter. For example, the length of the gas permeable membrane(s) of a cell encapsulation device is about 1 cm, 2 cm, 3 cm, 4 cm, 5 cm, 6 cm, 7 cm, 8 cm, 9 cm, 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, 1 meter. In some embodiments, length of the gas permeable membrane(s) of a cell encapsulation device is >1 meter.

In some embodiments, the inner space formed from the gas permeable membrane or membranes of the cell encapsulation device is partially filled with air. In some embodiments, 10%, 15%, 20%, 25%, 30%, 35%, 40%, 45%, 50%, 55%, 60%, 65%, 70%, 75%, 80%, 90%, 95%, >95% of the inner space is filled with air.

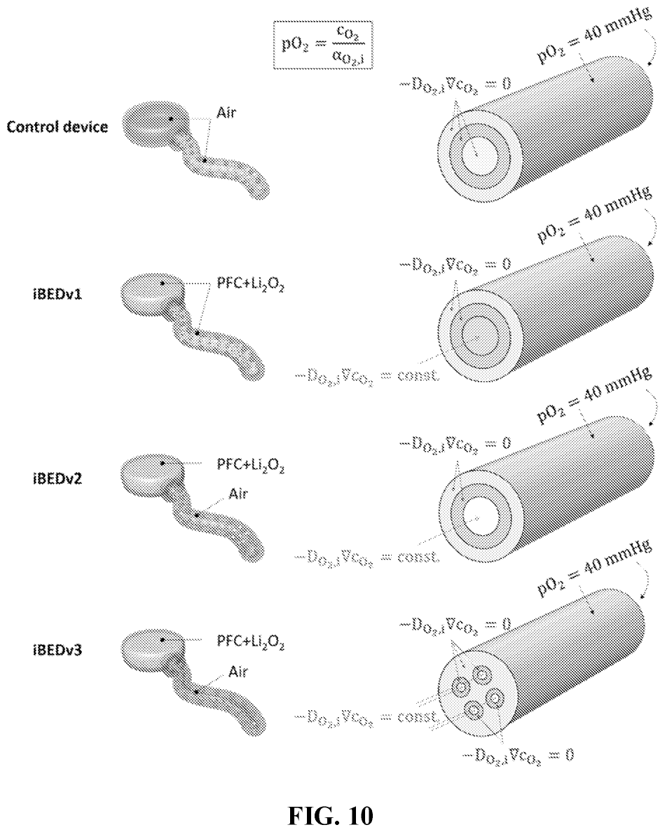

The openings of the gas permeable membranes of the cell encapsulation devices described herein optionally contain a fitting. The fitting, which is preferably permeable to oxygen and carbon dioxide, is suitable for coupling the cell encapsulation device to another component of a cell encapsulation system, for example, to a reservoir device comprising a source of oxygen for the encapsulated cells. In one embodiment, the cell encapsulation devices as described herein are coupled to a reservoir device as described herein (see e.g., FIGS. 10 and 38A). This reservoir device comprises a reservoir suitable for enclosing a liquid, a liquid contained within the reservoir, where the liquid is permeable to gas, and an oxygen-generating compound, where the oxygen-generating compound is immersed in the liquid contained within the reservoir as described supra. The various features of this reservoir device are described supra.

The cell encapsulation devices as described herein each comprise an outer hydrogel layer that is suitable for encapsulating living cells. In any embodiment, the outer surface of the gas permeable membrane is modified to accept the hydrogel layer. For example, in one embodiment, the outer surface of the gas permeable membrane is modified by salt leaching. In one embodiment, the outer surface of the gas permeable membrane is modified with polymer coating, e.g., a dopamine coating, to accept the hydrogel layer. In one embodiment, the outer surface of the gas permeable membrane is modified with salt leaching and polymer coating to accept the hydrogel layer.

The hydrogel layer of the cell encapsulation device is a cell growth matrix material. In any embodiment, the cell growth matrix material compromises a synthetic polymer selected from the group consisting of polyethylene glycol (PEG), poly(acrylic acid), poly(ethylene oxide), poly(vinyl alcohol), polyphosphazene, poly(hydroxyethyl methacrylate), triazole-zwitterion hydrogels, poly(sulfobetaine methacrylate), carboxybetaine methacrylate, poly[2-methacryloyloxyethyl phosphorylcholine, N-Hydroxyethyl acrylamide, copolymers thereof, derivatives thereof, and combinations thereof. In any embodiment, the cell growth matrix material compromises a natural polymeric material selected from the group consisting of collagen, elastin, fibrin, gelatin, gelatin-methacryloyl, silk fibroin, glycosaminoglycans, dextran, alginate, agarose, chitosan, bacterial cellulose, keratin, matrigel, decellularized hydrogels, and derivatives or combinations thereof. Exemplary hydrogel layer material comprise alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative, and combinations thereof.

In any embodiment, the hydrogel core comprises an alginate. In any embodiment, the alginate of the hydrogel core comprises SLG100 alginate. In any embodiment, the hydrogel core comprises a 0.5% to 4% (w/v) alginate solution. In any embodiment, the hydrogel core comprises a 1% to 3% (w/v) alginate solution. In any embodiment, the hydrogel core comprises a 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0% (w/v) alginate solution. In any embodiment, the hydrogel core comprises a 2% (w/v) alginate solution.

In any embodiment, the hydrogel layer comprises a thickness of 200-2000 μm. For example, and without limitation the hydrogel layer of the cell encapsulation device may comprise a thickness of 300-1900 μm, 400-1800 μm, 500-1700 μm, 600-1600 μm, 700-1500 μm, 800-1400 μm, 900-1300 μm, 1000-1200 μm, 300-400 μm, 300-500 μm, 300-600 μm, 300-700 μm, 300-800 μm, 300-900 μm, 300-1000 μm, 1000-2000 μm, 1000-1900 μm, 1000-1800 μm, 1000-1700 μm, 1000-1600 μm, 1000-1500 μm, 1000-1400 μm, 1000-1300 μm, 1000-1200 μm, or 1000-1100 μm.

In any embodiment, the cell growth hydrogel material further comprises one or more cell factors to enhance cell growth, differentiation, and/or survival of the cells positioned within the hydrogel material. Suitable cell factors include, without limitation glutamine, non-essential amino acids, epidermal growth factors, fibroblast growth factors, transforming growth factor/bone morphogenetic proteins, platelet derived growth factors, insulin growth factors, cytokines, fibronectin, laminin, heparin, collagen, glycosaminoglycan, proteoglycan, elastin, chitin derivatives, fibrin, and fibrinogen, FGF, bFGF, acid FGF (aFGF), FGF-2, FGF-4, EGF, PDGF, TGF-beta, angiopoietin-1, angiopoietin-2, placental growth factor (PlGF), VEGF, PMA (phorbol 12-myristate 13-acetate), and combinations thereof.

The hydrogel layer of the cell encapsulation device described herein comprises a preparation of cells. In any embodiment, the preparation of cells positioned or encapsulated in the hydrogel layer of the cell encapsulation device is a preparation of single cells. In any embodiment, the preparation of a cells is a preparation of cell aggregates. In any embodiment, the preparation of cells is a preparation of single cells and cell aggregates.

In any embodiment, the preparation of cells positioned or encapsulated in the hydrogel layer of the cell encapsulation device is a preparation of primary cells or a preparation of immortalized cells. In any embodiment, the preparation of cells is a preparation of mammalian cells. In any embodiment, the preparation of cells is a preparation of primate cells, rodent cells, canine cells, feline cells, equine cells, bovine cells, and porcine cells. In any embodiment, the preparation of cells is a preparation of human cells.

In any embodiment, the preparation of cells positioned or encapsulated in the hydrogel layer of the cell encapsulation device as described herein is a preparation of stem cells or stem cell derived cells. In any embodiment, the stem cells are pluripotent, multipotent, oligopotent, or unipotent stem cells. In any embodiment, the preparation of stem cells is selected from a preparation of embryonic stem cells, epiblast cells, primitive ectoderm cells, primordial germ cells, and induced pluripotent stem cells.

In any embodiment, the preparation of cells positioned or encapsulated in the hydrogel layer of the cell encapsulation device is a preparation of cells selected from a preparation of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme over-expressed feline kidney epithelial cells, myogenic cells, embryonic stem cell-derived neural progenitor cells, irradiated tumor cells, proximal tubule cells, neural precursor cells, astrocytes, genetically engineered cells.

In any embodiment, the preparation of cells positioned or encapsulated in the hydrogel layer of the cell encapsulation device are islet cells, stem cell-derived § cells, Factor VIII-producing fibroblasts, hepatocytes, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof, and combinations thereof.

In one embodiment, the cells produce one or more of insulin, coagulation factors, albumin, urea, human cytochrome P450 enzymes, and combinations thereof.

In any embodiment, the preparation of cells positioned in the hydrogel layer of the cell encapsulation device comprises a cell density of between 1×10³ to 1×10¹⁰ cells/mL. For example, the cell density may range from about 1×10³ cells/mL, 1×10⁴ cells/mL, 1×10⁵ cells/mL, 1×10⁶ cells/mL, 1×10⁷ cells/mL, 1×10⁸ cells/mL, or 1×10⁹ cells/mL up to about 1×10⁴ cells/mL, 1×10⁵ cells/mL, 1×10⁶ cells/mL, 1×10⁷ cells/mL, 1×10⁸ cells/mL, 1×10⁹ cells/mL or 1×10¹⁰ cells/mL, respectively.

In any embodiment, the preparation of cells positioned in the hydrogel layer of the cell encapsulation device as described herein are present at a concentration of 1%-40% v/v cells/hydrogel. For example, the cells are present in the hydrogel layer of the device at a concentration of about 5%-40% v/v, 10%-35% v/v, 15%-30% v/v, 20%-25% v/v, 5%-10% v/v, 5%-20% v/v, 5%-30% v/v, 35%-40% v/v, 30%-40% v/v, 25%-40% v/v, 20%-40% v/v cells/hydrogel.

In any embodiment, the preparation of cells positioned in the hydrogel layer of the cell encapsulation device as described herein is a preparation comprising islet cells that release insulin and glucagon. In any embodiment, the preparation of insulin producing cells is a preparation of human SC-β cells. In any embodiment, the preparation comprising islet cells and/or SC-β cells is a preparation of human islets and/or SC-β cells, porcine islets and/or SC-β cells, or rodent islets and/or SC-β cells.

In any embodiment, the preparation of cells comprises an islet density between 1×10³ to 6×10⁵ islet equivalents (IEQs)/mL. In any embodiment, the preparation of cells comprises an islet density between 1×10³ to 6×10⁴ islet equivalents (IEQs)/mL. For example, the islet equivalents may range from about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, or 5×10⁵ up to about 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, or 6×10⁵ islet equivalents (IEQs)/mL

In any embodiment, the hydrogel layer of the cell encapsulation device described herein further comprises a biologically active agent. Suitable biologically active agents include, without limitation, a protein, peptide, antibody or antibody fragment thereof, antibody mimetic, a nucleic acid, a small molecule, a hormone, a growth factor, an angiogenic factor, a cytokine, an anti-inflammatory agent, and any combination thereof. In one embodiment, the biologically active agent is an anti-inflammatory agent. Suitable anti-inflammatory agents include, without limitation, diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, salsalate, sulindac, tolmetin, and combinations thereof.

In any embodiment, the hydrogel layer of the cell encapsulation device described herein comprises one or more contrast agents to facilitate in vivo monitoring of the encapsulation device when implanted to determine device placement, location of the implanted device at some time point after implantation, health of the implanted device, deleterious effects on non-target cell types, inflammation, and/or fibrosis. Suitable contrast agents include, without limitation, nanoparticles, nanocrystals, gadolinium, iron oxide, iron platinum, manganese, iodine, barium, microbubbles, fluorescent dyes, and others known to those of skill in the art.

Methods of in vivo monitoring include but are not limited to confocal microscopy, 2-photon microscopy, high frequency ultrasound, optical coherence tomography (OCT), photoacoustic tomography (PAT), computed tomography (CT), magnetic resonance imaging (MRI), single photon emission computed tomography (SPECT), and positron emission tomography (PET). These alone or combined can provide useful means to monitoring the implantable device.

In any embodiment, the encapsulation device comprises an elongated configuration, a flattened configuration, a cylindrical configuration, a rectangular configuration, or combinations thereof. In any embodiment, the encapsulation device is configured for subcutaneous placement, transcutaneous placement, preperitoneal placement, transperitoneal placement, or intraperitoneal placement in a subject.

Another aspect of the present disclosure includes a system for providing oxygen to encapsulated cells. The system includes a reservoir device as described herein and any one of the cell encapsulation devices as described herein. The various features and characteristics of the reservoir device and cell encapsulation devices suitable for use in this system are described supra.

In accordance with this aspect of the disclosure, the reservoir device may be coupled to the cell encapsulation device. In one embodiment, the reservoir device is fluidically connected to the cell encapsulation device. In one embodiment, the reservoir device is in gaseous connection with the encapsulation device. In another embodiment, the encapsulation device is coupled to the reservoir device with a gas permeable fitting.

Another aspect of the present disclosure is directed to a method of delivering a therapeutic agent to a subject in need thereof. The method of delivery includes obtaining a cell encapsulation device or system as described herein, and implanting the cell encapsulation device or system in the subject. In a preferred embodiment, when carrying out the methods of delivering a therapeutic agent as described herein, the cell encapsulation device is a device that is coupled to a reservoir device suitable for providing oxygen to the cells of the cell encapsulation device.

Suitable subjects that can delivered a therapeutic agent in accordance with the methods described herein include, without limitation, a human, a mouse, a rat, a dog, a pig, a sheep, a cow, and a nonhuman primate.

In some embodiments, the subject in need of treatment thereof, is a subject having diabetes, and the method of delivering a therapeutic agent to the subject involves implanting a cell encapsulation device or system as described herein into the subject having diabetes.

In accordance with this embodiment, the one or more therapeutic agents of the cell encapsulation device or system is insulin, glucagon, or a combination thereof. In any embodiment, the insulin, glucagon, or combination thereof is released from a preparation of cells positioned in the hydrogel layer of the cell encapsulation device. In any embodiment, the preparation of cells comprises a preparation of islets. In any embodiment, the preparation of islets is a preparation of primate islets, rodent islets, canine islets, feline islets, equine islets, bovine islets, or porcine islets. In any embodiment, the preparation of islets is derived from a preparation of stem cells. In any embodiment, the preparation of stem cells is a preparation of pluripotent, multipotent, oligopotent, or unipotent stem cells. In any embodiment, the preparation of stem cells is a preparation comprising embryonic stem cells, epiblast cells, primitive ectoderm cells, primordial germ cells, and induced pluripotent stem cells.

In another embodiment, the subject in need of treatment thereof is a subject having a bleeding disorder, and the method of delivering a therapeutic agent to the subject involves implanting a cell encapsulation device or system as described herein into the subject having the bleeding disorder. In accordance with this embodiment, the bleeding disorder can be any bleeding disorder, such as hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency.

In accordance with this embodiment, the one or more therapeutic agents is a blood clotting factor released from a preparation of cells positioned in the hydrogel layer of the cell encapsulation device. In any embodiment, the preparation of cells comprises recombinant myoblasts, mesenchymal stromal cells, endothelial cells, induced pluripotent stem cell derived endothelial cells, induced pluripotent stem cell derived mesenchymal stromal cells, or a combination thereof. In any embodiment, the blood clotting factor is selected from the group consisting of Factor I, Factor II, Factor V, Factor VII, Factor VIII, Factor IX, Factor X, Factor XI, Factor XII, Factor XIII, and combinations thereof.

In another embodiment, the subject in need of treatment thereof is a subject having a lysosomal storage disorder, and the method of delivering a therapeutic agent to the subject involves implanting a cell encapsulation device or system as described herein into the subject having the lysosomal storage disorder. In any embodiment, the one or more therapeutic agents is an enzyme released from a preparation of cells positioned in the hydrogel layer of the cell encapsulation device. In any embodiment, the preparation of cells comprises hematopoietic stem cells, fibroblasts, myoblasts, Baby Hamster Kidney (BHK) cells, Chinese Hamster Ovary cells, Human Amniotic Epithelial (HAE) cells, mesenchymal stromal cells, induced pluripotent stem cell derived mesenchymal stromal cells, or combinations thereof. In any embodiment, the enzyme is selected from the group consisting of α-L-iduronidase, Iduronate-2-sulfatase, α-glucuronidase, Arylsulfatase A, alpha-Galactosidase A, and combinations thereof.

In another embodiment, the subject in need of treatment thereof is a subject having a neurological disorder, and the method of delivering a therapeutic agent to the subject involves implanting a cell encapsulation device or system as described herein into the subject having the neurological disorder. In any embodiment, neurological disorder is a sensory disorder. In any embodiment, the neurological disorder is selected from the group consisting of Parkinson's disorder, Alzheimer's disease, epilepsy, Huntington's disease, Amyotrophic lateral sclerosis, chronic pain, visual and hearing loss. In any embodiment, the one or more therapeutic agents is a therapeutic molecule released from a preparation of cells positioned in the hydrogel layer of the cell encapsulation device.

In accordance with this embodiment, the preparation of cells comprises choroid plexus cells, chromaffin cells, pheochomocytoma cell line PC12, human retinal pigment epithelial cells, NGF-secreting Baby Hamster Kidney (BHK) cells, myoblasts, human bone marrow-derived stem cells transfected with GLP-1, BDNF-producing fibroblasts, NGF-producing cells, CNTF-producing cells, adrenal chromaffin cells, BDNF-secreting Schwann cells, and combinations thereof. In any embodiment, the therapeutic molecule is selected from the group consisting of cerebrospinal fluid, extracellular fluid, levodopa, nerve growth factor (NGF), ciliary neurotrophic factor (CNTF), BLP-1, brain-derived neurotrophic factor (BDNF), vascular endothelial growth factor (VEGF), enkephalin, adrenaline, catecholamine, and combinations thereof.

In another embodiment, the subject in need of treatment thereof is a subject having cancer, and the method of delivering a therapeutic agent to the subject involves implanting a cell encapsulation device or system as described herein into the subject having cancer disorder. In any embodiment, the one or more therapeutic agents is a therapeutic molecule released from a preparation of cells positioned in the hydrogel layer of the cell encapsulation device. In any embodiment, the preparation of cells comprises IL-2-secreting myoblasts, endostatin-secreting cells, Chinese Hamster Ovary cells, and cytochrome P450 enzyme overexpressed feline kidney epithelial cells. In any embodiment, the therapeutic molecule is selected from IL-2, endostatin, cytochrome P450 enzyme, and combinations thereof.

In another embodiment, the subject in need of treatment thereof is a subject having chronic eye disease and the method of delivering a therapeutic agent to the subject involves implanting a cell encapsulation device or system as described herein into the subject having a chronic eye disease. In any embodiment, this method further involves administering one or more trophic factors to the subject to protect compromised retinal neurons and to restore neural circuits. In any embodiment, the chronic eye disease is selected from the group consisting of age-related macular degeneration, diabetic retinopathy, retinitis pigmentosa, glaucoma, macular telangiectasia, and combinations thereof.

In accordance with this embodiment, the one or more therapeutic agents is a therapeutic molecule released from a preparation of cells positioned in the hydrogel layer of the cell encapsulation device. In any embodiment, the preparation of cells comprises human retinal pigment epithelium cells, recombinant human retinal pigment epithelium cells, or a combination thereof. In any embodiment, the therapeutic molecule is selected from the group consisting of ciliary neurotrophic factor, antagonists against vascular endothelial growth factor and platelet-derived growth factor, and combinations thereof.

In another embodiment, the subject in need of treatment thereof is a subject having kidney failure and the method of delivering a therapeutic agent to the subject involves implanting a cell encapsulation device or system as described herein into the subject having kidney failure. In any embodiment, the one or more therapeutic agents is a therapeutic molecule released from a preparation of cells positioned in the hydrogel layer of the cell encapsulation device. In any embodiment, the preparation of cells comprises renal proximal tubule cells, mesenchymal stem cells, and a combination thereof.

In another embodiment, the subject in need of treatment thereof is a subject having chronic pain and the method of delivering a therapeutic agent to the subject involves implanting a cell encapsulation device or system as described herein into the subject having chronic pain. In any embodiment, chronic pain is chronic pain caused by degenerative back and knee, neuropathic back and knee, or cancer. In any embodiment, the one or more therapeutic agents is a therapeutic molecule released from a preparation of cells positioned in the hydrogel layer of the cell encapsulation device. In any embodiment, the preparation of cells comprises chromaffin cells, neural precursor cells, mesenchymal stem cells, astrocytes, and genetically engineered cells, or a combination thereof. In any embodiment, the therapeutic molecule is selected from the group consisting of catecholamine, opioid peptides, enkephalins, and combinations thereof.

In accordance with aspects of the disclosure related to delivering a therapeutic agent to a subject in need thereof, the method of delivering the therapeutic agent involves implanting a cell encapsulation device or system as described herein using a laparoscopic procedure. In some embodiments, the cell encapsulation device or system is implanted preperitoneally, intraperitoneally, transperitoneally, transcutaneously, or subcutaneously. In some embodiments, implanting the cell encapsulation device as described herein involves suturing the device or system to a body wall of the subject. In some embodiments, implanting the device or system involves anchoring the device to a body wall of the subject via a transabdominal portal. In some embodiments, implanting the cell encapsulation device involves wrapping the delivery device or system in omentum of the subject. In some embodiments, implanting the cell encapsulation device involves positioning the device in a cavity between the liver and the diaphragm. In some embodiments, implanting the device or system involves anchoring the device to the diaphragm.

In accordance with the methods of delivering a therapeutic agent to a subject in need thereof or treating one of the various conditions as described above, the method further involves retrieving the cell encapsulation device from the subject when no longer needed or when the device needs replacement. Accordingly, these methods can further involve implanting a replacement cell encapsulation device after the initial device is retrieved. In another embodiment. In any embodiment, the method of retrieving the cell encapsulation device involves retrieving one or more components of the implanted encapsulation device or system from the subject. For example, in one embodiment, the reservoir device of the cell encapsulation device or system is retrieved and replaced in the subject. In another embodiment, the cell encapsulation component of the device is retrieved and replaced in the subject.

### EMBODIMENTS

The invention provides also the following non-limiting embodiments.

Embodiment 1 of the present disclosure is directed to a reservoir device for providing oxygen to encapsulated cells. The device comprising a reservoir; an oxygen-generating compound contained within the reservoir; and a liquid contained within the reservoir, wherein the liquid is permeable to gas.

Embodiment 2 is the reservoir device of embodiment 1, wherein the reservoir further comprises an opening.

Embodiment 3 is the reservoir device of embodiment 2, wherein the opening further comprises a fitting.

Embodiment 4 is the reservoir device of any one of embodiments 2 or 3, wherein the opening is capped with a gas permeable membrane.

Embodiment 5 is the reservoir device of embodiment 4, wherein the gas permeable membrane is permeable to oxygen and carbon dioxide.

Embodiment 6 is the reservoir device of any one of embodiments 4-5, wherein the gas permeable membrane comprises a material selected from the group consisting of polydimethylsiloxane (PDMS), silicone-based membranes, and polytetrafluoroethylene (PTFE).

Embodiment 7 is the reservoir device of any one of embodiments 1-6, wherein the oxygen-generating compound reacts with carbon dioxide to release oxygen.

Embodiment 8 is the reservoir device of any one of embodiments 1-7, wherein the oxygen-generating compound comprises lithium peroxide, sodium peroxide, potassium peroxide, potassium superoxide, and combinations thereof.

Embodiment 9 is the reservoir device of any one of embodiment 1-8, wherein the liquid comprises one or liquids selected from the group consisting of perfluorocarbon (PFC) oil, mineral oil, silicone oil.

Embodiment 10 is the reservoir device of any one of embodiments 1-9, wherein the reservoir comprises a wall.

Embodiment 11 is the reservoir device of embodiment 10, wherein the wall is impermeable to solids, liquids, and gases.

Embodiment 12 is the reservoir device of any one of embodiments 10-11, wherein the wall comprises a biocompatible material.

Embodiment 13 is the reservoir device of embodiment 11, wherein the wall comprises a biocompatible resin, a medical grade alloy, titanium, titanium alloy, stainless steel, cobalt chrome alloy, nickel titanium alloy, gold, platinum, silver, iridium, tantalum, tungsten, and combinations thereof.

Embodiment 14 is the reservoir device of any one of embodiments 1-13, wherein the device comprises a flattened configuration, a cylindrical configuration, a rectangular configuration or combinations thereof.

Embodiment 15 is the reservoir device of any one of embodiments 1-14, wherein the device is configured for subcutaneous placement in a subject, transperitoneal placement in a subject, intraperitoneal placement in a subject, and/or preperitoneal placement in a subject.

Embodiment 16 is the reservoir device of any one of embodiments 1-15, wherein the device comprises a plurality of openings.

Embodiment 17 is the reservoir device of any one of embodiments 1-16, wherein the device has the dimensions of 10-80 mm diameter and 3-30 mm height.

Embodiment 18 is the reservoir device of any one of embodiments 1-17, wherein the oxygen-generating compound is submerged in the liquid.

Embodiment 19 is the reservoir device of any one of embodiments 1-18, wherein the reservoir is configured to be refillable with oxygen-generating compound and/or liquid.

Embodiment 20 is directed to a cell encapsulation device comprising: a permeable membrane enclosing an inner space; an oxygen-generating compound filling the inner space; and a liquid contained within the inner space, wherein the liquid is permeable to gas.

Embodiment 21 is the encapsulation device of embodiment 20, wherein the permeable membrane further comprises an opening.

Embodiment 22 is the encapsulation device of embodiment 21, wherein the opening further comprises a fitting.

Embodiment 23 is the encapsulation device of any one of embodiments 20 and 21, wherein the opening is capped with a gas permeable fitting.

Embodiment 24 is the encapsulation device of embodiment 23, wherein the gas permeable fitting is permeable to oxygen and carbon dioxide.

Embodiment 25 is the encapsulation device of any one of embodiments 23-24, wherein the gas permeable fitting comprises a material selected from the group consisting of polydimethylsiloxane (PDMS), silicone-based membranes, and polytetrafluoroethylene (PTFE).

Embodiment 26 is the encapsulation device of any one of embodiments 20-25, wherein the oxygen-generating compound reacts with carbon dioxide to release oxygen.

Embodiment 27 is the encapsulation device of any one of embodiments 20-26, wherein the oxygen-generating compound is lithium peroxide, sodium peroxide, potassium peroxide, potassium superoxide, and combinations thereof

Embodiment 28 is the encapsulation device of any one of embodiment 20-27, wherein the liquid comprises one or liquids selected from the group consisting of perfluorocarbon (PFC) oil, mineral oil, silicone oil.

Embodiment 29 is the encapsulation device of any one of embodiments 20-28, wherein the permeable membrane comprises a material selected from the group consisting of polydimethylsiloxane (PDMS), silicone-based membranes, and polytetrafluoroethylene (PTFE).

Embodiment 30 is the encapsulation device of any one of embodiments 20-29, further comprising a hydrogel layer on an outer surface of the permeable membrane.

Embodiment 31 is the encapsulation device of embodiment 20-30, wherein the hydrogel layer is selected from the group consisting of alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative and combinations thereof.

Embodiment 32 is the encapsulation device of any one of embodiments 30-31, wherein the hydrogel layer comprises a thickness of 200-2000 μm.

Embodiment 33 is the encapsulation device of any one of embodiments 30-32, wherein the outer surface of the permeable layer is modified to accept the hydrogel layer.

Embodiment 34 is the encapsulation device of embodiment 33, wherein the outer surface of the permeable layer is modified with salt leaching to accept the hydrogel layer.

Embodiment 35 is the encapsulation device of any one of embodiments 31-34, wherein the hydrogel layer contains cells.

Embodiment 36 is the encapsulation device of embodiment 35, wherein the cells comprise cells selected from the group consisting of islet cells, stem cell-derived § cells, Factor VIII-producing fibroblasts, hepatocytes, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof, and combinations thereof.

Embodiment 37 is the encapsulation device of embodiment 36, wherein the cells produce one or more of insulin, coagulation factors, albumin, urea, human cytochrome P450 enzymes.

Embodiment 38 is the encapsulation device of any one of embodiments 35-37, wherein the cells are at a concentration of 1%-40% v/v cells/hydrogel.

Embodiment 39 is the encapsulation device of any one of embodiments 20-38, wherein the device comprises an elongated configuration, a flattened configuration, a cylindrical configuration, a rectangular configuration, or combinations thereof.

Embodiment 40 is the encapsulation device of any one of embodiments 20-39, wherein the device is configured for subcutaneous placement, transcutaneous placement, preperitoneal placement, transperitoneal placement, or intraperitoneal placement in a subject.

Embodiment 41 is the encapsulation device of any one of embodiments 20-40, wherein the device comprises a plurality of openings.

Embodiment 42 is directed to an encapsulation device comprising: a gas permeable membrane enclosing an inner space, wherein the inner space is at least partially filled with air; and a hydrogel layer on an outer surface of the permeable membrane.

Embodiment 43 is the encapsulation device of claim 42, wherein the permeable membrane further comprises an opening.

Embodiment 44 is the encapsulation device of claim 43, wherein the opening further comprises a fitting.

Embodiment 45 is the encapsulation device of any one of claims 43-44, wherein the opening is capped with a gas permeable fitting.

Embodiment 46 is the encapsulation device of claim 45, wherein the gas permeable fitting is permeable to oxygen and carbon dioxide.

Embodiment 47 is the encapsulation device of any one of claims 45-46, wherein the gas permeable fitting comprises a material selected from the group consisting of polydimethylsiloxane (PDMS), silicone-based membranes, and polytetrafluoroethylene (PTFE).

Embodiment 48 is the encapsulation device of any one of claims 42-47, wherein the hydrogel layer comprises a material selected from the group consisting of alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative, and combinations thereof.

Embodiment 49 is the encapsulation device of any one of claims 42-48, wherein the hydrogel layer comprises a thickness of 200-2000 μm.

Embodiment 50 is the encapsulation device of any one of claims 42-49, wherein the outer surface of the permeable layer is modified to accept the hydrogel layer.

Embodiment 51 is the encapsulation device of claim 50, wherein the outer surface of the permeable layer is modified with salt leaching to accept the hydrogel layer and/or dip coating in a polymer solution (e.g. PMMA in a DMF solution, PVDF-HFP in acetone solution) followed by drying to accept the hydrogel layer.

Embodiment 52 is the encapsulation device of any one of claims 42-51, wherein the hydrogel layer contains cells.

Embodiment 53 is the encapsulation device of claim 52, wherein the cells comprise cells selected from the group consisting of islet cells, stem cell-derived § cells, Factor VIII-producing fibroblasts, hepatocytes, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof, and combinations thereof.

Embodiment 54 is the encapsulation device of claim 54, wherein the cells produce one or more of insulin, coagulation factors, albumin, urea, human cytochrome P450 enzymes.

Embodiment 55 is the encapsulation device of any one of claims 53-54, wherein the cells are at a concentration of 1%-40% v/v cells/hydrogel.

Embodiment 56 is the encapsulation device of any one of claims 42-55, wherein the device comprises an elongated configuration, a flattened configuration, a cylindrical configuration, a rectangular configuration, or combinations thereof

Embodiment 57 is the encapsulation device of any one of claims 42-56, wherein the device is configured for subcutaneous placement, transcutaneous placement, preperitoneal placement, transperitoneal placement, or intraperitoneal placement in a subject.

Embodiment 58 is the encapsulation device of any one of claims 42-57, wherein the device comprises a plurality of openings.

Embodiment 60 is directed to an encapsulation device comprising: an elongated gas permeable membrane comprising a first opening and a second opening; and a hydrogel layer on an outer surface of the gas permeable membrane, wherein the membrane encloses an inner space, wherein the inner space is at least partially filled with air, wherein the gas permeable membrane is twisted helically about a longest axis of the membrane.

Embodiment 61 is the encapsulation device of embodiment 60, further comprising a fitting, wherein the first opening and second opening are connected to the fitting.

Embodiment 62 is the encapsulation device of embodiment 61, wherein the fitting is permeable to oxygen and carbon dioxide.

Embodiment 63 is the encapsulation device of any one of embodiments 61-62, wherein the fitting comprises a material selected from the group consisting of polydimethylsiloxane (PDMS), silicone-based membranes, and polytetrafluoroethylene (PTFE).

Embodiment 64 is the encapsulation device of any one of embodiments 60-63, wherein the hydrogel layer comprises a material selected from the group consisting of alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative, and combinations thereof.

Embodiment 65 is the encapsulation device of any one of embodiments 60-64, wherein the hydrogel layer comprises a thickness of 200-2000 μm.

Embodiment 66 is the encapsulation device of any one of embodiments 60-65, wherein the outer surface of the permeable layer is modified to accept the hydrogel layer.

Embodiment 67 is the encapsulation device of embodiment 66, wherein the outer surface of the permeable layer is modified with salt leaching to accept the hydrogel layer and/or dip coating in a polymer solution (e.g. PMMA in a DMF solution, PVDF-HFP in acetone solution) followed by drying to accept the hydrogel layer.

Embodiment 68 is the encapsulation device of any one of embodiments 60-67, wherein the hydrogel layer contains cells.

Embodiment 69 is the encapsulation device of embodiment 68, wherein the cells comprise cells selected from the group consisting of islet cells, stem cell-derived § cells, Factor VIII-producing fibroblasts, hepatocytes, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof, and combinations thereof.

Embodiment 70 is the encapsulation device of embodiment 69, wherein the cells produce one or more of insulin, coagulation factors, albumin, urea, human cytochrome P450 enzymes.

Embodiment 71 is the encapsulation device of any one of embodiments 68-70, wherein the cells are at a concentration of 1%-40% v/v cells/hydrogel.

Embodiment 72 is the encapsulation device of any one of embodiments 60-71, further comprising: a second elongated gas permeable membrane comprising a third opening and a fourth opening; and a second hydrogel layer on an outer surface of the second gas permeable membrane, wherein the second membrane encloses a second inner space, wherein the second inner space is at least partially filled with air, wherein the second membrane is twisted helically about a longest axis of the first membrane.

Embodiment 73 is the encapsulation device of embodiment 72, wherein the third opening and fourth opening are connected to a fitting.

Embodiment 74 is the encapsulation device of any one of embodiments 60-73, wherein the device further comprises a plurality of elongated gas permeable membranes.

Embodiment 75 is the encapsulation device of any one of embodiments 60-74, wherein the device is configured for subcutaneous placement, transcutaneous placement, preperitoneal placement, transperitoneal placement, or intraperitoneal placement in a subject.

Embodiment 76 is a system for providing oxygen to encapsulated cells, the system comprising: a reservoir device of any one of embodiments 1-19; and an encapsulation device of any one of claims 20-41.

Embodiment 77 is the system of embodiment 76, wherein the reservoir device is in fluid connection with the encapsulation device.

Embodiment 78 is the system of embodiment 76, wherein the reservoir device is in gas connection with the encapsulation device.

Embodiment 79 is the system of any one of embodiments 76-78, wherein the encapsulation device is selectively coupled to the reservoir device.

Embodiment 80 is the system of any one of embodiments 76-79, wherein the encapsulation device is selectively coupled to the reservoir device with a gas permeable fitting.

Embodiment 81 is a system for providing oxygen to encapsulated cells, the system comprising: a reservoir device of any one of embodiments 1-19; and an encapsulation device of any one of embodiments 42-58.

Embodiment 82 is the system of embodiment 81, wherein the reservoir device is in gas connection with the encapsulation device.

Embodiment 83 is the system of any one of embodiments 81-82, wherein the encapsulation device is selectively coupled to the reservoir device.

Embodiment 84 is the system of any one of embodiments 81-83, wherein the encapsulation device is selectively coupled to the reservoir device with a gas permeable fitting.

Embodiment 85 is a system for providing oxygen to encapsulated cells, the system comprising: a reservoir device of any one of embodiments 1-19; and an encapsulation device of any one of claims 60-75.

Embodiment 86 is the system of embodiment 85, wherein the reservoir device is in gas connection with the encapsulation device.

Embodiment 87 is the system of any one of embodiments 85-86, wherein the encapsulation device is selectively coupled to the reservoir device.

Embodiment 88 is the system of any one of embodiments 85-87, wherein the encapsulation device is selectively coupled to the reservoir device with a gas permeable fitting.

Embodiment 89 is a method of treating a subject, the method comprising: obtaining a reservoir device of any one of embodiments 1-19 and/or an encapsulation device of any one of embodiments 20-41, 42-58, 60-75; and implanting the reservoir device and/or encapsulation device in the subject.

Embodiment 90 is the method of embodiment 89, wherein the subject is selected from the group consisting of a human, a mouse, a rat, a dog, a pig, a sheep, a cow, and a nonhuman primate.

Embodiment 91 is the method of any one of embodiments 89-90, wherein the reservoir device and/or encapsulation device is implanted subcutaneously.

Embodiment 92 is the method of any one of embodiments 89-91, wherein the reservoir device and encapsulation device are selectively coupled.

Embodiment 93 is the method of any one of embodiments 89-92, wherein the subject suffers from diabetes.

Embodiment 94 is the method of any one of embodiments 89-93, wherein the encapsulation device provides insulin to the subject.

Embodiment 95 is the method of any one of embodiments 89-94, wherein the encapsulation device provides insulin for at least 15, 30, 45, 60, 75, or 90 days.

Embodiment 96 is the method of any one of embodiments 89-95, wherein further comprising replacing the reservoir device.

Embodiment 97 is a device for encapsulated cells, the device comprising: a reservoir comprising an opening; an oxygen-generating compound contained within the reservoir; a gas-permeable membrane fitted to the opening; and a hydrogel disposed on an outer surface of the gas-permeable membrane.

Embodiment 98 is the device of embodiment 97, further comprising a liquid within the reservoir.

Embodiment 99 is the device of embodiment 98, wherein the liquid comprises one or liquids selected from the group consisting of perfluorocarbon (PFC) oil, mineral oil, silicone oil.

Embodiment 100 is the device of any one of embodiments 97-99, wherein the oxygen-generating compound is submerged in the liquid.

Embodiment 101 is the device of any one of embodiments 97-99, wherein the oxygen-generating compound reacts with carbon dioxide to release oxygen.

Embodiment 102 is the device of any one of embodiments 97-101, wherein the oxygen-generating compound comprises lithium peroxide, sodium peroxide, potassium peroxide, potassium superoxide, and combinations thereof.

Embodiment 103 is the device of any one of embodiments 97-102, wherein the gas permeable membrane is permeable to oxygen and carbon dioxide

Embodiment 104 is the device of any one of embodiments 97-103, wherein the gas permeable membrane comprises a material selected from the group consisting of polydimethylsiloxane (PDMS), silicone-based membranes, and polytetrafluoroethylene (PTFE).

Embodiment 105 is the device of any one of embodiments 97-104, wherein the reservoir comprises a wall.

Embodiment 106 is the device of embodiment 105, wherein the wall is impermeable to solids, liquids, and gases.

Embodiment 107 is the device of any one of embodiments 105-106, wherein the wall comprises a biocompatible material.

Embodiment 108 is the device of any one of embodiments 105-107, wherein the wall comprises a biocompatible resin, a medical grade alloy, titanium, titanium alloy, stainless steel, cobalt chrome alloy, nickel titanium alloy, gold, platinum, silver, iridium, tantalum, tungsten, and combinations thereof.

Embodiment 109 is the device of any one of claims 97-108, wherein the device comprises a flattened configuration, a cylindrical configuration, a rectangular configuration or combinations thereof.

Embodiment 110 is the device of any one of embodiments 97-109, wherein the device is configured for subcutaneous placement in a subject, preperitoneal placement in a subject, transcutaneous placement in a subject, intraperitoneal placement in a subject, and/or preperitoneal placement in a subject.

Embodiment 111 is the device of any one of embodiments 97-110, wherein the device comprises a plurality of openings.

Embodiment 112 is the device of any one of embodiments 97-111, wherein the device has the dimensions of 10-80 mm diameter and 3-30 mm height.

Embodiment 113 is the device of any one of embodiments 97-112, wherein the hydrogel layer is selected from the group consisting of alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative and combinations thereof.

Embodiment 114 is the device of any one of embodiments 97-113, wherein the hydrogel layer comprises a thickness of 200-2000 μm.

Embodiment 115 is the device of any one of embodiments 97-114, wherein the outer surface of the gas permeable membrane is modified to accept the hydrogel layer.

Embodiment 116 is the device of embodiment 115, wherein the outer surface of the permeable layer is modified with salt leaching to accept the hydrogel layer.

Embodiment 117 is the device of any one of claims 97-116, wherein the hydrogel layer contains cells.

Embodiment 118 is the device of embodiment 117, wherein the cells comprise cells selected from the group consisting of islet cells, stem cell-derived § cells, Factor VIII-producing fibroblasts, hepatocytes, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof, and combinations thereof.

Embodiment 119 is the device of any one of embodiments 117-118, wherein the cells produce one or more of insulin, coagulation factors, albumin, urea, human cytochrome P450 enzymes.

Embodiment 120 is the device of any one of embodiments 117-118, wherein the cells are at a concentration of 1%-40% v/v cells/hydrogel.

Embodiment 121 is the method of treating a subject, the method comprising: obtaining a device of any one of embodiments 97-120; and implanting the device in the subject.

Embodiment 122 is the method of embodiment 121, wherein the subject is selected from the group consisting of a human, a mouse, a rat, a dog, a pig, a sheep, a cow, and a nonhuman primate.

Embodiment 123 is the method of any one of embodiments 121-122, wherein the device is implanted subcutaneously.

Embodiment 124 is the method of any one of embodiments 121-122, wherein the device is implanted transcutaneously.

Embodiment 125 is the method of any one of embodiments 121-124, wherein the subject suffers from diabetes.

Embodiment 126 is the method of any one of embodiments 121-125, wherein the device provides insulin to the subject.

Embodiment 127 is the method of any one of embodiments 121-126, wherein the device provides insulin for at least 15, 30, 45, 60, 75, or 90 days.

### EXAMPLES

The examples below are intended to exemplify the practice of embodiments of the disclosure but are by no means intended to limit the scope thereof.

### Materials and Methods of Examples 1-7

Chemicals. Sodium chloride (NaCl), calcium chloride dihydrate (CaCl₂·2H₂O), barium chloride dihydrate (BaCl₂·2H₂O), calcium sulfate dihydrate (CaSO₄·2H₂O), lithium carbonate (Li₂CO₃), and D-glucose were purchased from Sigma-Aldrich. Lithium peroxide (Li₂O₂) was purchased from Alfa Aesar. Perfluorocarbon (PFC) oil (Krytox® GPL103) was purchased from DuPont. Polydimethylsiloxane (PDMS, Sylgard 184) was purchased from Dow Corning. Ultrapure sodium alginate (Pronova SLG100) was purchased from NovaMatrix. The Dental LT Clear resin and Flexible resin for 3D printing were purchased from Formlabs. Water was deionized to 18.2 MΩ·cm with a Synergy UV purification system (Millipore Sigma).

Animals. 8-week-old male C57BL/6J mice were purchased from the Jackson Laboratory (Bar Harbor, ME). Male Sprague-Dawley rats (weight ˜300 g) were purchased from Charles River Laboratories (Wilmington, MA). 6-month-old female Gottingen Minipigs were purchased from Marshall Bioresources (North Rose, NY). All animal procedures were approved by the Cornell Institutional Animal Care and Use Committee.

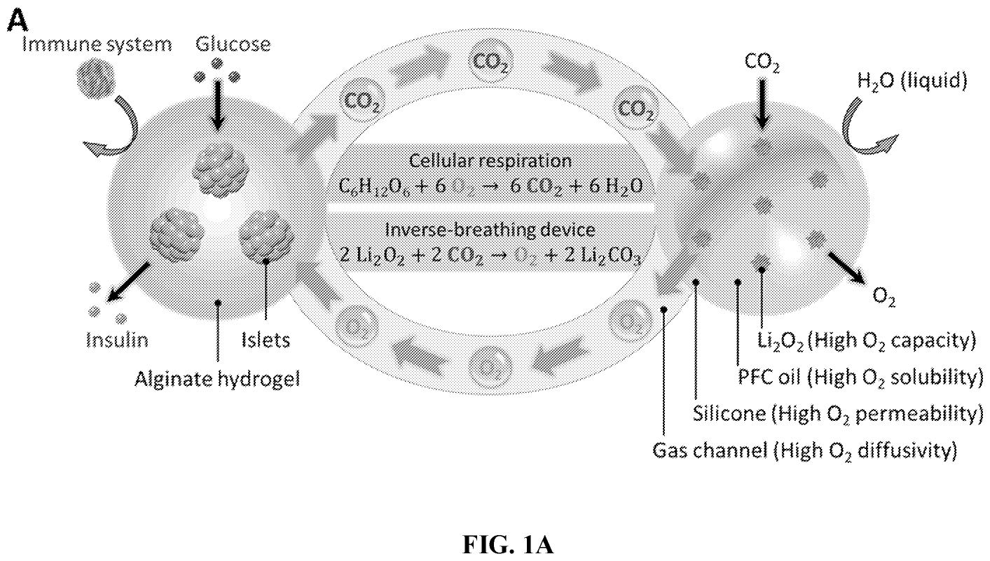

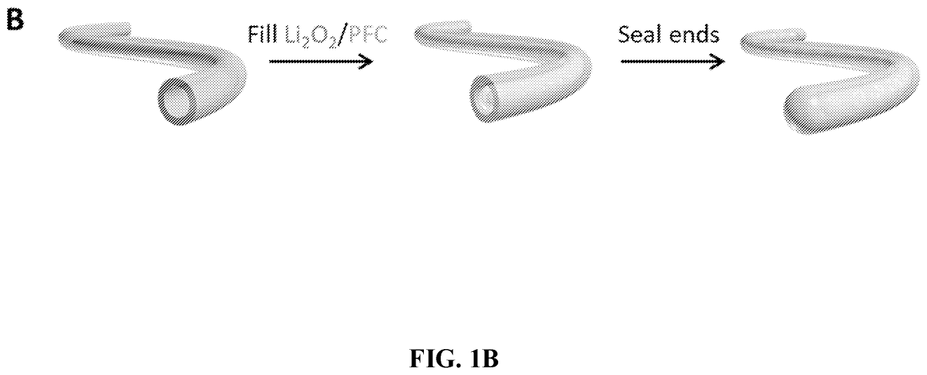

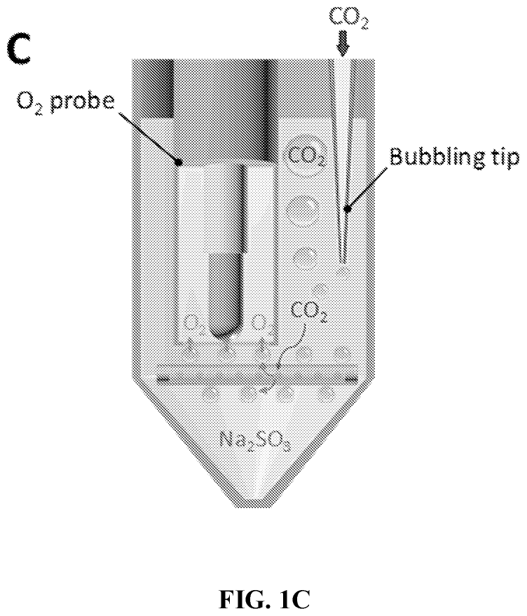

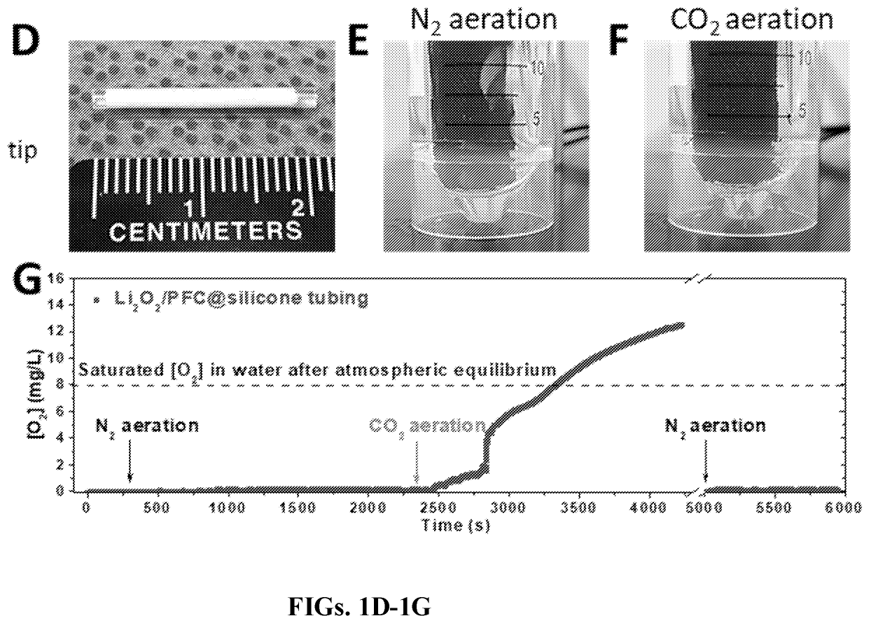

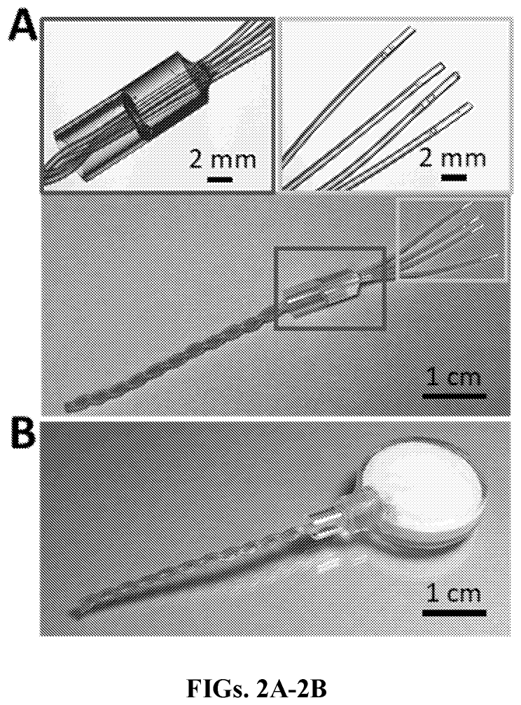

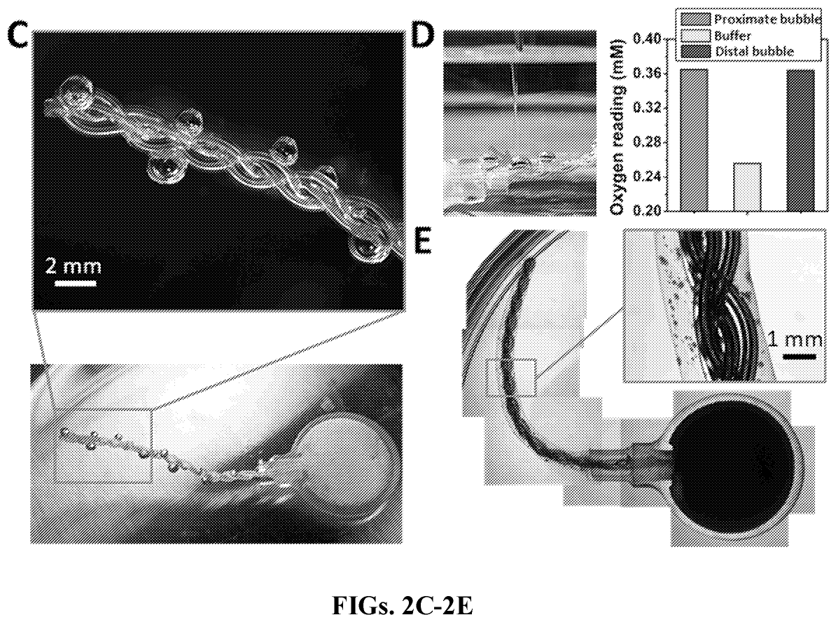

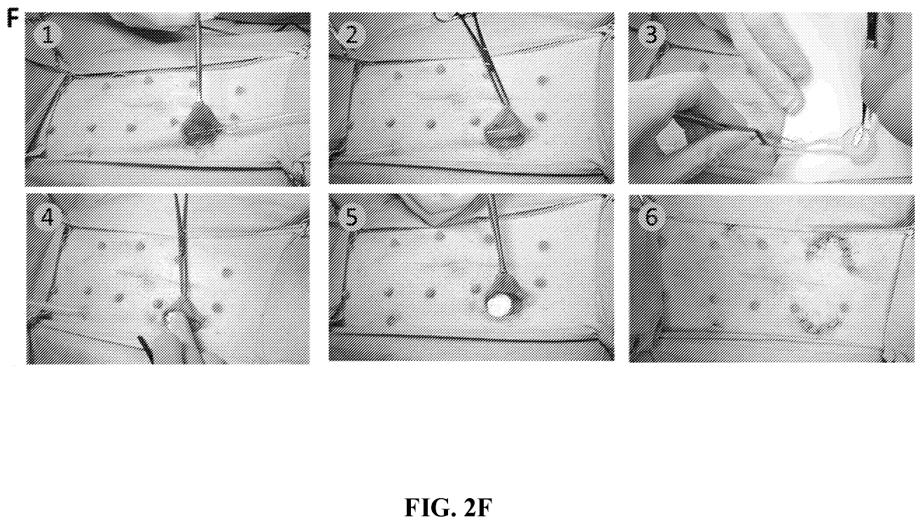

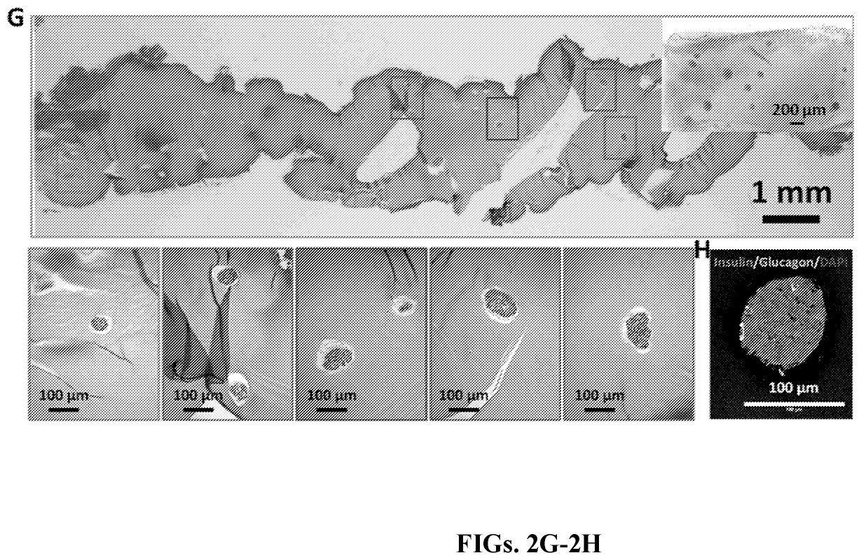

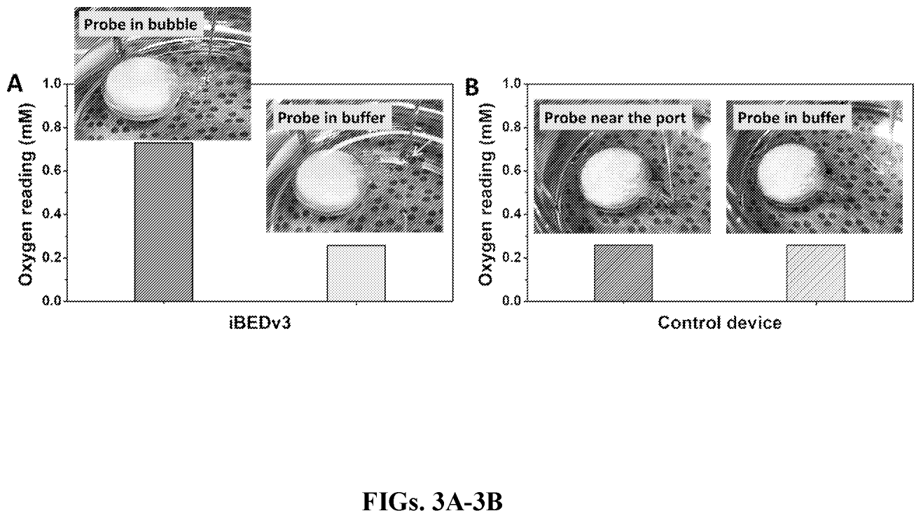

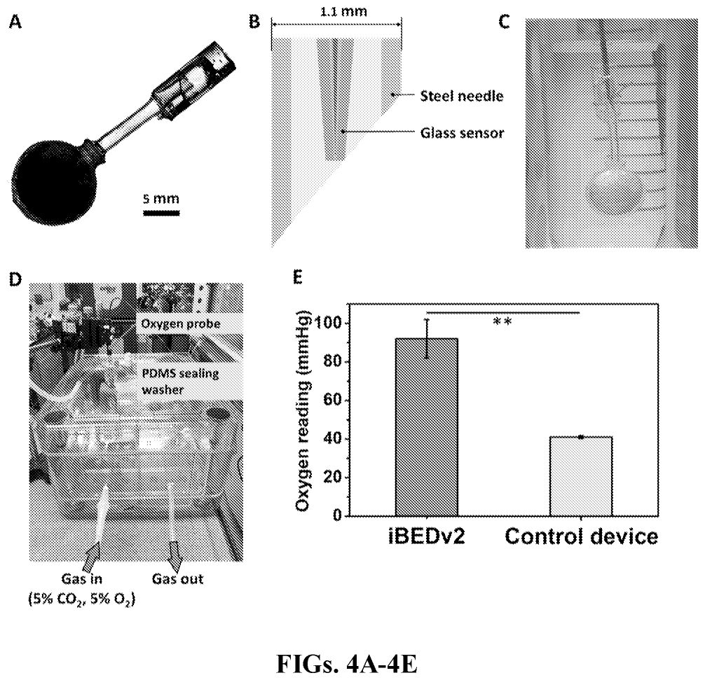

Characterizations. H&E staining images were taken using an Aperio Scanscope (CS2). Optical and fluorescent microscope images were taken using a digital inverted microscope (EVOS FL). Stereo microscope images were taken using a stereomicroscope (Olympus SZ61). Confocal images were taken using a laser scanning confocal microscope (LSM 710). Three different Clark-type O₂ sensors were used for O₂ measurement. An O₂ sensor (Vernier) equipped with a wide tip (˜12 mm) was used to test the CO₂ responsiveness of the Li₂O₂/PFC@silicone tubing construct (FIG. 1C and FIGS. 1E-1G). An O₂ microsensor (OX-100, Unisense) equipped with a 100 μm glass tip was used to measure the pO₂ in the bubbles generated around the device (FIG. 2D and FIG. 3). An O₂ needle sensor (OX-N, Unisense) equipped with a 1.1×40 mm piercing needle was used to measure the pO₂ in the lumen of device (FIG. 4).

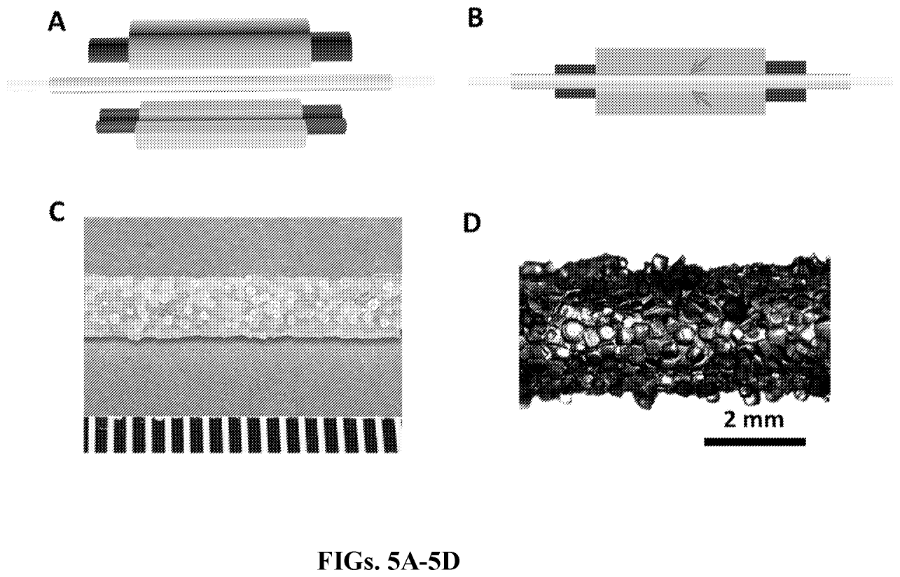

Surface roughness modification of silicone tubing by salt leaching. A two-part hollow mold with an inner diameter of 3 mm was printed on a 3D printer (Form 2, Formlabs) using a flexible resin. A thin layer of NaCl salt was placed at the bottom of each half piece, and then curable PDMS resin was applied on the top of NaCl salt. After the PDMS resin settled down and permeated through the NaCl salt layer, a silicone tubing (Specialty Manufacturing, Inc., Class VI, i.d.˜1.47 mm, o.d.˜1.96 mm) was placed between two half round pieces (FIG. 5A). The mold was fastened and put into an oven at 60° C. After curing for 3 h, the tubing was unmolded. The coated tubing was soaked in hot water to leach out the NaCl.

Fabrication of different cell encapsulation devices designs and the corresponding control devices. The cell encapsulation devices described in the examples are referred to as Inverse Breathing Encapsulation Devices (iBEDs). To fabricate the Li₂O₂/PFC@silicone tubing device, the silicone tubing (2 cm length, i.d.˜1.47 mm, o.d.˜1.96 mm) surface was modified via the salt-leaching method (described in another section). Then, the surface-roughness modified silicone tubing was incubated in dopamine solution (2 mg/mL in 10 mM tris buffer, pH 8.5) overnight to create a hydrophilic external polydopamine coating (Lee et al., “Mussel-inspired Surface Chemistry for Multifunctional Coatings,” Science 318:426-430 (2007), which is hereby incorporated by reference in its entirety). Next, the lumen was filled with 20% (w/w) Li₂O₂/PFC (or 20% w/w LiCO₃/PFC in controls) and sealed with PDMS resin at both ends. CaSO₄ was then deposited onto the tubing surface by dipping the tubing in a 1% (w/v) CaSO₄/ethanol suspension and allowing excess ethanol to evaporate. Finally, a layer (˜500 μm) of ultrapure sodium alginate (Pronova SLG100) was created by filling a cylindrical mold with alginate solution and inserting the tubing into the mold; alginate crosslinking occurred by the outward radial diffusion of Ca²⁺ ions from the scaffold surface. Constructs which contained islets or cells were fabricated by premixing the alginate solution with the desired cells before application onto the modified silicone tubing.

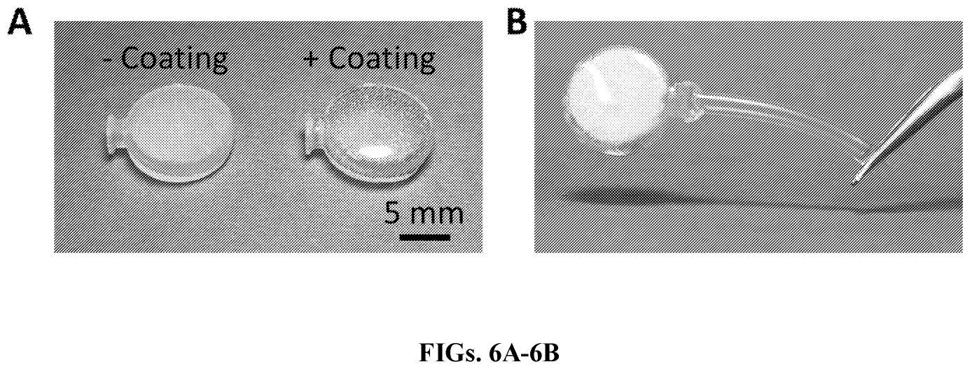

To fabricate iBEDv1, a reserve tank (12 mm diameter, 4 mm thickness) was printed on a 3D printer (Form 2, Formlabs) using a Dental LT Clear resin. A thin layer of liquid uncured resin was then painted onto the tank and cured at 70° C. under blue light (405 nm) for 20 min, providing a smoother surface and patching over any potential gaps or defects between the printed layers (FIG. 6A). The tank was filled with 20% (w/w) Li₂O₂/PFC (or 20% w/w Li₂CO₃/PFC in controls) and attached with a 1.5 cm Li₂O₂/PFC@silicone tubing construct (fabricated as previously described) via an adapter. The adapter was comprised of an unmodified silicone tubing (4 mm in length) filled and cured with PDMS resin. To facilitate attachment to the terminal tank, a layer of PDMS resin was applied around the adapter and cured to seal the gap between adapter and tank neck. 500 IEQ of rat islets distributed in a layer of ˜500 μm-thick alginate at a density of ˜6250 IEQ/mL were incorporated in the iBEDv1.

Fabrication of iBEDv2 was identical to that of iBEDv1, with the exception that in iBEDv2, the lumen of the silicone tubing was left empty. To fabricate an iBEDv2 to support lumen pO2 measurements, an unmodified silicone tubing was attached to the reserve tank. The free end of the tubing was then capped with a half-sealed larger silicone tubing (i.d.˜3.35 mm), and the gap between two cylinders was sealed with PDMS resin (FIG. 4A).

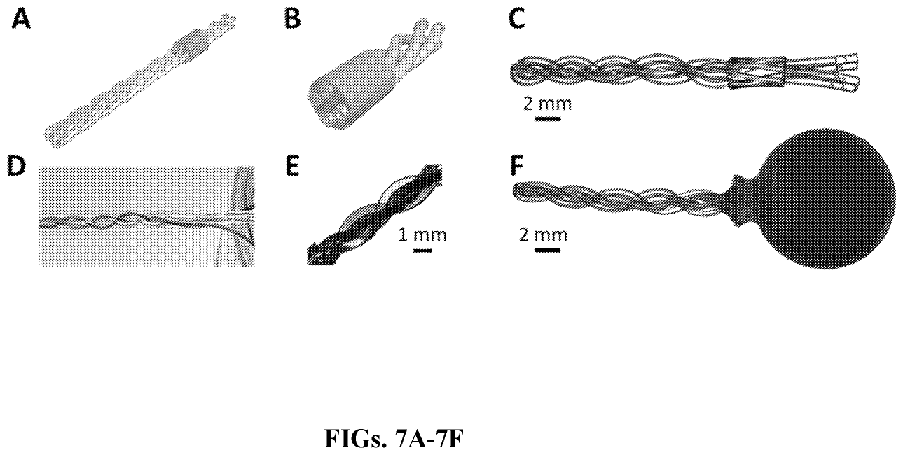

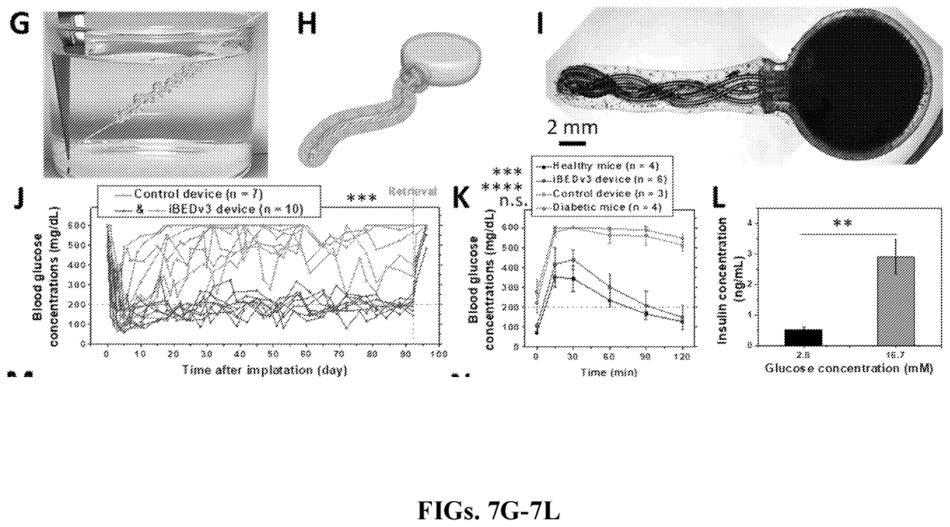

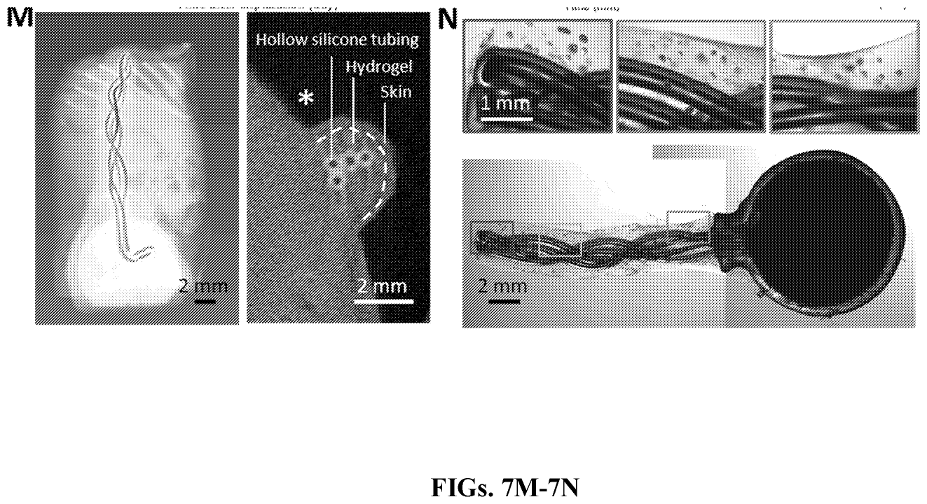

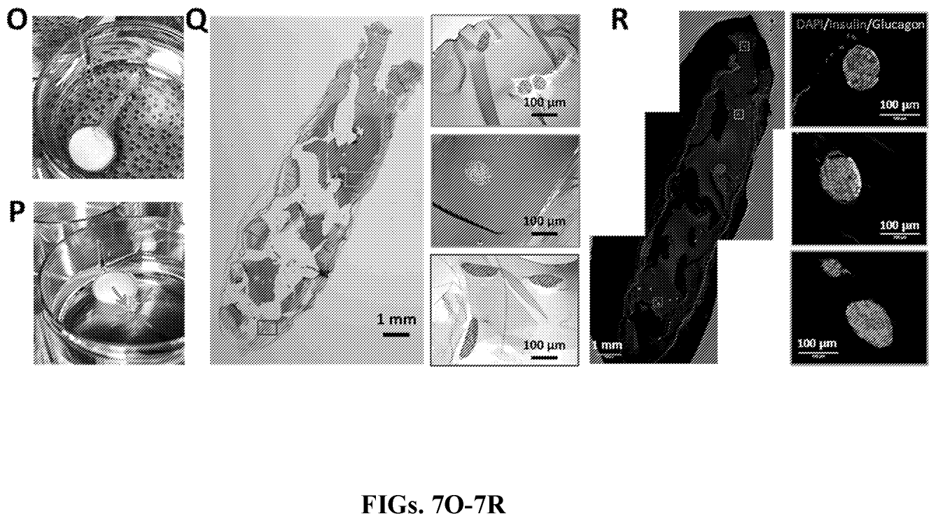

To fabricate iBEDv3, two hollow silicone tubes (i.d.˜0.34 mm, o.d.˜0.64 mm) were twisted and folded at one end and sealed at the other, creating a four-thread twisted structure resembling the simulated quadruple helix (FIG. 7A). An adapter, which connected the terminal tank to the cell encapsulation domain, was fabricated as follows: the twisted silicone hollow tubes were fit through a short, larger hollow silicone tube (4 mm length, i.d.˜1.47 mm) and fixed into place by filling the gap among these four small tubing and the large tubing with PDMS resin. 500 IEQ of rat islets distributed in a layer of ˜500 μm-thick alginate at a density of ˜8330 IEQ/mL were incorporated in the iBEDv3.

To fabricate the scaled-up third generation iBED design (iBEDv3S), an enlarged reserve tank (22 mm diameter, 8 mm thickness) was 3D printed as described previously. The length of twisted silicone tubing was extended to 40 mm. The twisted silicone tubing was fixed in a larger adapter (12 mm length, i.d.˜3.35 mm) as described in iBEDv3. The adapter was half-filled with PDMS prior to curing, providing protection from mechanical stress to the alginate near the tank (FIG. 2A). 1500 IEQ of rat islets were incorporated in the iBEDv3S at a same density as in iBEDv3.

To fabricate the controls of different iBED designs, each experimental control device is identical to its corresponding iBED design, with the exception that the Li₂O₂/PFC filling was replaced with Li₂CO₃/PFC which does not produce oxygen.

All devices were sterilized using a hydrogen peroxide plasma sterilizer before the cell encapsulation procedure.

Rat islet isolation and purification. Sprague-Dawley rats (˜300 g) were used for harvesting islets. The rats were anesthetized using 3% isoflurane in O₂, and the anesthesia was maintained throughout the whole surgery. Briefly, the bile duct was cannulated, and the pancreas was distended with 10 mL 0.15% Liberase (Roche) in M199 media (Gibco). The pancreas was digested at 37° C. circulating water bath for ˜28 mins (digestion time varied slightly for different batches of Liberase). The digestion was stopped by adding cold M199 media with 10% FBS (Gibco). After vigorously shaking, the digested pancreases were washed twice with media (M199+10% FBS), filtered through a 450 m sieve, and then suspended in a Histopaque 1077 (Sigma)/M199 media gradient and centrifuged at 1,700 RCF with 0 break and 0 acceleration for 17 min at 4° C. This gradient centrifugation step was repeated for higher purity. Finally, the islets were collected from the gradient and further isolated by a series of gravity sedimentations, in which each top supernatant was discarded after 4 min of settling. Islet equivalent number (IEQ) of purified islets was counted by reported IEQ conversion factors (Buchwald et al., “Quantitative Assessment of Islet Cell Products: Estimating the Accuracy of the Existing Protocol and Accounting for Islet Size Distribution,” Cell Transplant. 18:1223-1235 (2009), which is hereby incorporated by reference in its entirety). Islets were then washed once with islet culture media (RPMI 1640+10% FBS+10 mM HEPES+1% penicillin/streptomycin) and cultured in this medium overnight before further use.

Hypoxic cell culture. Hypoxic cell culture was performed in a New Brunswick™ Galaxy® CO-170 incubator which has dynamic control over CO₂ and O₂ levels. The incubator was equipped with both compressed CO₂ and N₂ gas cylinders. The CO-170 incubator controlled internal pO₂, when set below ambient levels, by modulating N₂ inflow.

In vitro cell viability study. INS-1 cells were cultured in RPMI 1640 medium (Gibco) supplemented with 2 mM glutamine (Gibco), 1 mM sodium pyruvate (Gibco), 10 mM HEPES (Gibco), 10% FBS (Gibco), 50 μM β-mercaptoethanol (Gibco), and 1% penicillin/streptomycin (Gibco). INS-1 cells (2.5 million cells/mL) were incorporated into Li₂O₂/PFC@silicone constructs and Li₂CO₃/PFC@silicone controls and incubated in a hypoxic incubator with 1% O₂, 5% CO₂. After 24 h of incubation, the cells were stained with a LIVE/DEAD™ viability/cytotoxicity kit (Invitrogen).

Immunochemical detection of islet hypoxia. Li₂O₂/PFC@silicone tubing devices containing encapsulated rat islets were cultured in a hypoxic incubator with 1% O₂, 5% CO₂. After 24 h of incubation, pimonidazole (Hypoxyprobe) was added to the culture media at a final concentration of 200 μmol/L, and then the samples were returned to the incubator for an additional 2 h of incubation. The samples were then fixed in 10% formalin and were permeabilized with 0.5% Triton X-100 for 30 min at room temperature. After cells were blocked for unspecific binding in 5% donkey serum, the samples were incubated overnight at 4° C. with FITC-conjugated anti-pimonidazole mouse IgG1 monoclonal antibody (Hypoxyprobe, 1:200). The formation of pimonidazole-protein adducts were analyzed by a fluorescent microscope (EVOS fl) and a confocal microscope (LSM 710).

Morphology and immunohistochemistry of islets and retrieved samples. Li₂O₂/PFC@silicone tubing devices containing encapsulated rat islets were cultured in a hypoxic incubator with 1% O₂, 5% CO₂. After 24 h of incubation, the samples were then fixed in 10% formalin, embedded in paraffin, and sectioned by Cornell's Histology Core Facility. 5 μm sections were stained with hematoxylin and eosin. For immunofluorescent insulin and glucagon staining, paraffin-embedded sections were deparaffinized in xylene and sequentially rehydrated in 100% ethanol, 95% ethanol, 75% ethanol, and PBS. Slides were then boiled in citric acid buffer (10 mM citric acid, 0.05% Tween 20, pH 6.0) for 30 min for antigen retrieval. After blocking with 5% donkey serum, primary rabbit anti-rat insulin (Abcam, ab63820, 1:200) and mouse anti-rat glucagon (Abcam, ab10988, 1:200) antibodies were applied and incubated overnight at 4° C. After washing with PBS, Alexa Fluor 594-conjugated goat anti-rabbit IgG (Thermofisher, A11037, 1:400) and Alexa Fluor 488-conjugated donkey anti-mouse IgG (Thermofisher, A21202, 1:400) were applied and incubated for 60 min. Finally, slides were washed with PBS, applied with antifade/DAPI, and covered with glass coverslips.

Samples retrieved from animals were fixed in 10% formalin, embedded in paraffin, and sectioned by Cornell's Histology Core Facility. 5 μm sections were stained with hematoxylin and eosin. Insulin and glucagon staining were performed as described above. α-Smooth Muscle-Cy3 (Sigma-Aldrich, C6198, 1:200) was used for myofibroblast staining. Anti-mouse CD68-AF488 (BioLegend, Cat #137012, 1:200) was used for macrophage staining. Primary antibody rabbit anti-CD3 (Abcam, ab5690, 1:100) and second antibody Alexa Fluor 594-conjugated goat anti-rabbit IgG (Thermofisher, A11037, 1:400) were used for T cell staining.

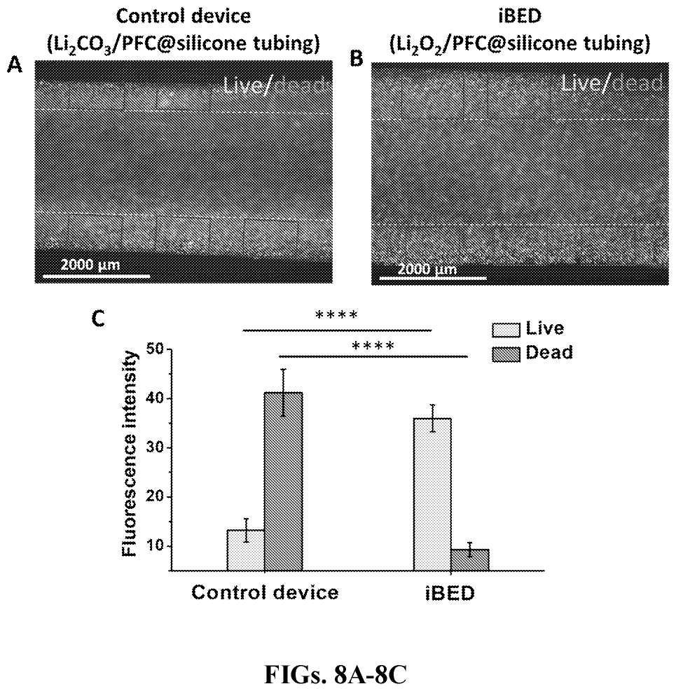

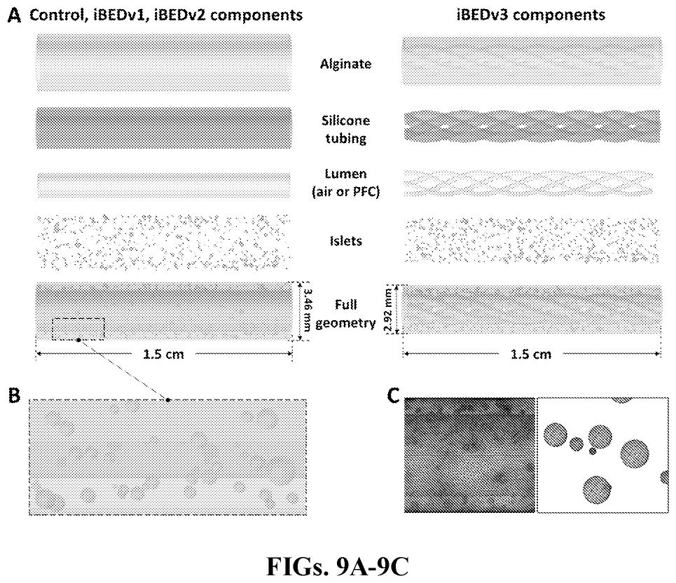

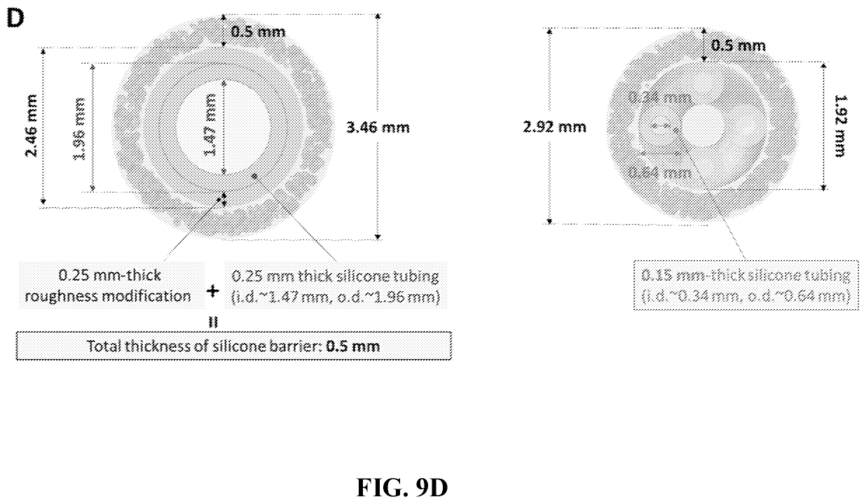

Computational modeling. A finite element model was developed to study the impact of device design on islet oxygenation. The control, iBEDv1, and iBEDv2 geometries were analogous, featuring concentric cylinders representing the alginate, PDMS tubing, and lumen content (i.e. air in the control and iBEDv2, and PFC in iBEDv1) respectively; the iBEDv3 geometry was implemented as a cylinder, representing the alginate, and an internal quadruple helix representing the silicone tubing (i.d.˜0.34 mm, o.d.˜0.64 mm) and internal air (FIG. 8B and FIG. 9). Transport in the terminal storage tank was not considered, rather, O₂ production was implemented at a constant rate of 3.2×10⁻⁹ mol/min (4.7×10⁻⁶ mol/d), converted to a surface flux by dividing by surface area at the lumen-tank interface (FIG. 10). This production rate was determined as follows: (1) iBEDv2 devices were incubated in a hypoxia incubator with 5% O₂, 5% CO₂ for 2 weeks, then the devices were transferred into a pre-equilibrated hypoxia chamber (5% O₂, 5% CO₂, FIG. 4D) to measure the lumen pO₂ in devices with a Clark electrode, providing values of 85-99 mmHg (FIG. 4E); (2) an in silico iBEDv2 model was developed which omitted the encapsulated islets and a production rate was determined such that the model-predicted lumen pO₂ was on the conservative end of this range at ˜90 mmHg. Note, this value is consistent with the expected O₂ generation rate from 50 mg Li₂O₂ (the amount loaded in the iBEDv2 tank) constitutively produced over the observed period of O₂ generation of ˜4 months. It was assumed that a constant pO₂ of 40 mmHg was maintained at all locations of the device-host boundary (FIG. 10).

Islet size and seeding were carefully considered. Optical microscope images from three rat islet isolations (n=1,660 islets) were collected, and islet perimeters were traced manually using ImageJ and converted into effective diameters (d_{eff}) by the area method. These were sorted by size, an exact cumulative frequency curve of d_{eff} was then calculated, and finally fit to a lognormal cumulative distribution function; a robust fit was found with shape factor σ=0.36 and scale factor m=119.7 (R²=0.998; the probability density function for a lognormal distribution is given by Equation 3):

$$f ( {d}_{eff} ) = \frac{1}{{d}_{eff} σ \sqrt{2 π}} exp ( - \frac{{ln ( \frac{{d}_{eff}}{m} )}^{2}}{2 {σ}^{2}} ) \qquad Equation 3$$

This is comparable with distributions in other species and is consistent with theoretical models of islet growth kinetics (Buchwald et al., “Quantitative Assessment of Islet Cell Products: Estimating the Accuracy of the Existing Protocol and Accounting for Islet Size Distribution,” Cell Transplant. 18:1223-1235 (2009), which is hereby incorporated by reference in its entirety)). A total of 500 IEQ of islets with diameters selected randomly from this distribution were included each iteration.

Oxygen concentration (c_{O}_{₂}) was governed by the steady state diffusion-reaction mass balance equation with assumed negligible convection (Equation 4):

$${D}_{{O}_{2} , i} ( \frac{1}{r} \frac{\partial }{\partial r} ( r \frac{\partial {c}_{{O}_{2}}}{\partial r} ) + \frac{1}{{r}^{2}} \frac{{\partial }^{2} {c}_{{O}_{2}}}{\partial {θ}^{2}} + \frac{{\partial }^{2} {c}_{{O}_{2}}}{\partial {z}^{2}} ) = {R}_{{O}_{2} , i} \qquad Equation 4$$

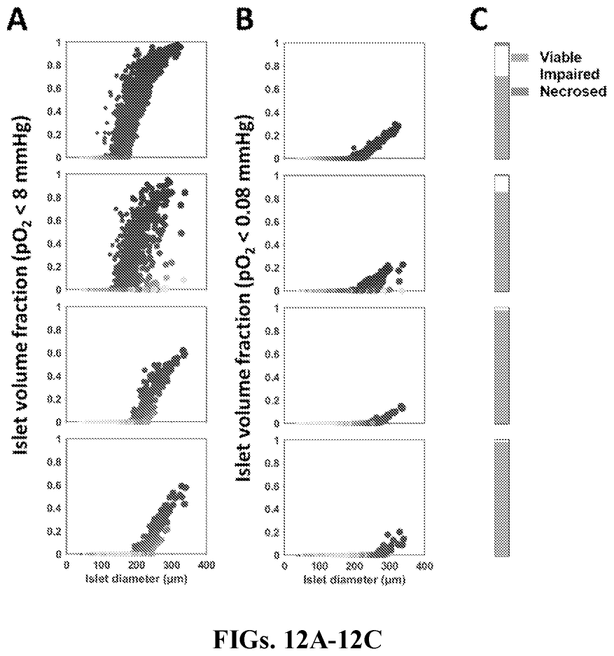

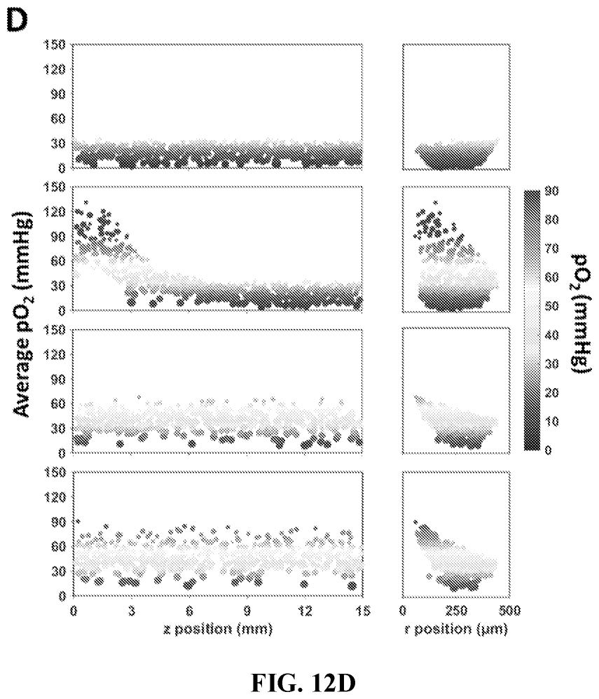

In Equation 4, diffusivity (D_{O}_{₂}_{,i}) represents the diffusivity of O₂ in domain i (i.e. in silicone, PFC, air, alginate, or islets) at 37° C., and were selected according to values reported in the literature: D_{O}_{₂}_{,silicone}=3.25×10⁻⁹ m²/s (Markov et al., “Variation in Diffusion of Gases Through PDMS Due to Plasma Surface Treatment and Storage Conditions,” Biomed. Microdevices 16:91-96 (2014), which is hereby incorporated by reference in its entirety), D_{O}_{₂}_{,PFC}=5.6×10⁻⁹ m²/s (O'Brien et al., “Diffusion Coefficients of Respiratory Gases in a Perfluorocarbon Liquid,” Science: 153-155 (1982), which is hereby incorporated by reference in its entirety), D_{O}_{₂}_{,air}=1.8×10⁻⁵ m²/s (Chapman et al., “The Mathematical Theory of Non-Uniform Gases: An Account of the Kinetic Theory of Viscosity, Thermal Conduction and Diffusion in Gases,” Cambridge university press (1990), which is hereby incorporated by reference in its entirety) D_{O}_{₂}_{aginate}=2.7×10⁻⁹ m²/s (Mehmetoglu et al., “Oxygen Diffusivity in Calcium Alginate Gel Beads Containing Gluconobacter Suboxydans,” Artif Cells Blood Substit. Immobil. Biotechnol. 24:91-106 (1996), which is hereby incorporated by reference in its entirety), and D_{O}_{₂}_{,isiets}=2.0×10⁻⁹ m²/s (Avgoustiniatos et al., “Measurements of the Effective Diffusion Coefficient of Oxygen in Pancreatic Islets,” Ind. Eng. Chem. Res. 46:6157-6163 (2007), which is hereby incorporated by reference in its entirety). Finally, R_{O}_{₂}_{,i} represents O₂ consumption in domain i. R_{O}_{₂}_{,i} was zero in all domains except the islets, where it was implemented using Michaelis-Menten kinetics and a step-down function to simulate the lack of O₂ consumption in necrosed cells (Equation 5), as in other models (Buchwald et al., “Glucose-stimulated Insulin Release: Parallel Perifusion Studies of Free and Hydrogel Encapsulated Human Pancreatic Islets,” Biotechnol. Bioeng. 115:232-245 (2018) and Suszynski et al., “Oxygenation of the Intraportally Transplanted Pancreatic Islet,” J. Diabetes Res. 2016 (2016), which are hereby incorporated by reference in their entirety):

$${R}_{{O}_{2} , islets} = \{ \begin{matrix}0 , & {c}_{{O}_{2}} < {c}_{necrosis} \\ \frac{{R}_{max} {c}_{{O}_{2}}}{{c}_{{O}_{2}} + {K}_{m}} , & {c}_{{O}_{2}} \geq {c}_{necrosis}\end{matrix} \qquad Equation 5$$

where Rₘₐₓ=0.034 mol/(m³ s) represents the maximum O₂ uptake rate of rat islets (Avgoustiniatos et al., “Measurements of the Effective Diffusion Coefficient of Oxygen in Pancreatic Islets,” Ind. Eng. Chem. Res. 46:6157-6163 (2007), which is hereby incorporated by reference in its entirety), Kₘ=1.0×10⁻³ mol/m³ represents the half-maximal coefficient (obtained from studies of mitochondrial respiration) (Wilson et al., “The Oxygen Dependence of Mitochondrial Oxidative Phosphorylation Measured by a New Optical Method for Measuring Oxygen Concentration,” J. Biol. Chem. 263:2712-2718 (1988), which is hereby incorporated by reference in its entirety), and c_{necrosis}=1.0×10⁻⁴ mol/m³ represents a viability threshold (Dulong and Legallais., “A Theoretical Study of Oxygen Transfer Including Cell Necrosis for the Design of a Bioartificial Pancreas,” Biotechnol. Bioeng. 96:990-998 (2007) and Wijaranakula, “Solubility of Interstitial Oxygen in Silicon.” Appl. Phys. Lett. 59:1185-1187 (1991), which are hereby incorporated by reference in their entirety). Solving Equation 4 for c_{O}_{₂} yields spatial O₂ distributions; c_{O}_{₂} was converted into pO₂ in presented data (FIG. 11 and FIG. 12) by dividing c_{O}_{₂} by Bunsen solubility values for each respective domain, or via the ideal gas law in the case of air (Equation 6):

$${pO}_{2} = \frac{{c}_{{O}_{2}}}{{α}_{{O}_{2} , i}} \qquad Equation 6$$

where α_{O}_{₂}_{,silicone}=7.3×10⁻⁵ mol/(m³ Pa) (Wijaranakula, “Solubility of Interstitial Oxygen in Silicon.” Appl. Phys. Lett. 59:1185-1187 (1991), which is hereby incorporated by reference in its entirety), α_{O}_{₂}_{,PFC}=1.9×10⁻⁴ mol/(m³ Pa) (Lewis, “Eliminating Oxygen Supply Limitations for Transplanted Microencapsulated Islets in the Treatment of Type 1 Diabetes,” Thesis, Massachusetts Institute of Technology (2008) and Tham et al., “Diffusion Coefficients of Molecular Oxygen, Molecular Nitrogen, and Carbon Dioxide in Fluorinated Ethers,” J. Chem. Eng. Data 18:411-412 (1973), which are hereby incorporated by reference in their entirety), α_{O}_{₂}_{,air}=3.9×10⁻⁴ mol/(m³ Pa), α_{O}_{₂}_{,aiginate}=9.3×10⁻⁶ mol/(m³ Pa) Lewis, “Eliminating Oxygen Supply Limitations for Transplanted Microencapsulated Islets in the Treatment of Type 1 Diabetes,” Thesis, Massachusetts Institute of Technology (2008), which is hereby incorporated by reference in its entirety) and α_{O}_{₂}_{,isiets}=7.36×10⁻⁶ mol/(m³ Pa) (Lewis, “Eliminating Oxygen Supply Limitations for Transplanted Microencapsulated Islets in the Treatment of Type 1 Diabetes,” Thesis, Massachusetts Institute of Technology (2008) and Avgoustiniatos et al., “Measurements of the Effective Diffusion Coefficient of Oxygen in Pancreatic Islets,” Ind. Eng. Chem. Res. 46:6157-6163 (2007), which are hereby incorporated by reference in their entirety). At all internal interfaces, a partition coefficient (K_{i|j}) represented by the Bunsen solubility ratios was implemented (Equation 7):

$${K}_{i \vert j} = \frac{{α}_{{O}_{2} , i}}{{α}_{{O}_{2} , j}} \qquad Equation 7$$

Meshes were generated using the “Free Tetrahedral” tool in COMSOL Multiphysics 5.4 (Burlington, MA) with the following settings: maximum element size=400 μm, minimum element size=0.3 μm, curvature factor=0.4, resolution of narrow regions=1.1, and maximum element growth rate=1.125. The fitting of the lognormal distribution parameters was performed with the Curve Fitting toolbox in MATLAB (Natick, MA). Random size selection and seeding was performed also performed with MATLAB. For each design, the size and seeding of the simulated islets was re-randomized and the results were re-computed 100 times (i.e. a Monte Carlo simulation) using the COMSOL Livelink for MATLAB software. Results in FIGS. 11C-11E, FIG. 11G, and FIG. 12D were collected from one iteration selected at random; results in FIG. 11F and FIG. 12A-12C were collected from the aggregate of all iterations.

EPR O₂ distribution mapping. All pO₂ maps were obtained using a JIVA-25 instrument (O2M Technologies, LLC) at the JDRF supported “Oxygen Measurement Core” facility. JIVA-25 operates at 720 MHz. The trityl radical, OX063-d24 (methyl-tris[8-carboxy-2,2,6,6-tetrakis[(2-hydroxyethyl]benzo[1,2-d:4,5-d′]bis[1,3]dithiol-4-yl]-trisodium salt), was obtained from the N.N. Vorozhtsov Novosibirsk Institute of Organic Chemistry. The devices (control or iBEDv3 devices) were added to 3 mL of solution (either 1% gelatin or 100 mM CaCl₂) in water) in a glass tube (VWR, 15×85 mm). 72 mM OX063-d24 was added to the solution to achieve a final concentration of 1 mM. N₂, CO₂, and mixed-gas (5% CO₂, 5% O₂, 90% N₂) cylinders for these studies were purchased from Medox, Inc. The gas mixture of 5% CO₂, 95% N₂ was prepared using MC gas mass flow controllers (Alicat Scientific, model numbers MC-50SCCM-D/5M and MC-5SCCM-D/5M). Deoxygenation was achieved using 100% N₂ using a submerged tubing at the rate of 10 sccm. Dissolved gas equilibration was achieved by bubbling of gas mixture (either 5% CO₂, 95% N₂ or 5% CO₂, 5% O₂, 90% N₂) at the rate of 6 sccm. Thereafter, gas mixture overhead flow was at the rate of 6 sccm. Average pO₂ measurements of the whole system (solution and device) were performed using inversion recovery electron spin echo (IRESE) sequence with the following parameters: pulse lengths 60 ns, 8 phase cycles scheme with FID suppression, spin echo delay 500 ns, 80 logarithmically spaced delays from 350 ns-40 μs, 55 us repetition time. The curves were fitted using single exponential recovery to extract spin-lattice relaxation rates R₁ (1/T₁) values that were converted to pO₂. The pO₂ calibration details are as follows: O₂ relaxation rate at 0 mmHg 0.115×10⁶ s⁻¹, the slope 124.6×10⁻⁶ mmHg/s⁻¹. pO₂ imaging was performed using IRESE sequence with the following parameters: pulse lengths 60 ns, 8 phase cycles scheme with FID suppression, spin echo delay 500 ns, equal solid angle spaced 654 projections, 67 baselines, 1.5 G/cm gradient, 8 time delays from 350 ns-30 μs, 45 us repetition time, overall 10 min image duration. Images were reconstructed using filtered back-projection in isotropic 64×64×64 cube with 0.66 mm voxel linear size.

In vivo X-ray micro-computed tomography (Micro-CT) imaging in mice. Micro-CT analyses were performed on a SkyScan 1276 system (Bruker). The mice were anesthetized using 3% isoflurane in oxygen, and then were placed in an exchangeable animal cassette and maintained the anesthesia throughout the whole scanning process. During the scans, the X-ray source was set to a voltage of 80 kV and a current of 200 μA. The images were obtained using a binning mode of 2×2. The resolution of the obtained images is around 20 μm per pixel. The obtained images were reconstructed into 2D transverse cross sections using NRecon software (version 1.7.4.2). Subsequently, 3D reconstruction was performed using Avizo software (version 8.1.1). A segmentation process was conducted to visualize the maintained hollow structure of the silicone tubing based on the different absorption contrasts between the gas phase, and the silicone tubing, hydrogel, and mouse tissue.

Implantation and retrieval in mice. 8-week-old male C57BL/6J mice were used for device implantations. To create diabetic mice, healthy mice were administered an IP injection of freshly prepared STZ (Sigma-Aldrich) solution (22.5 mg/mL in 100 mM sodium citrate buffer, pH 4.5) at a dosage of 150 mg STZ/kg mouse. The BG levels of all mice were retested prior to transplantation. Only mice with non-fasted blood glucose levels above 350 mg/dL were considered as diabetic. The diabetic mice were anesthetized with 3% isoflurane in O₂ and their dorsal skin were shaved and sterilized using betadine and 70% ethanol.

A lateral transverse incision (0.5 cm for a tubing device, 1.2 cm for a device featured a terminal tank) was made on the dorsum. A pocket (0.5 cm×2.5 cm for a tubing construct, 1.2 cm×3 cm for a device featured a terminal tank) was created in the SC space using a blunt surgical tool. The tubing device was inserted into the pocket, and the incision was closed using a nylon suture. Reflex wound clips (Roboz) were applied in certain cases when deemed necessary.

For retrieval, some devices were excised along with the surrounding skin and fibrotic capsule. For the retrieval of other devices, a 1 cm incision was made along the implanted device, and the devices were pulled out after cutting open the surround fibrotic capsule. The incision was closed using 5-0 absorbable polydioxanone (PDS II) sutures.

Implantation and retrieval in Gottingen minipigs. Each animal received two implants in the ventral deep SC space. Two subjects received one iBEDv3S and one control device (i.e. the device without the inverse-breathing feature) with retrieval at one month, one received two iBEDv3S devices with retrieval also at one month, and one received two iBEDv3S devices with retrieval at 2 months.

The minipigs were premedicated with glycopyrrolate and butorphanol, induced with propofol, and anesthetized with isoflurane in O₂. The ventral skin of the minipig was shaved and prepared for sterile surgery. A 3 cm diameter semicircular incision was made using a scalpel, and a deep subcutaneous pocket was created for the terminal tank of iBEDv3S. Another 1 cm long transverse incision was made roughly 10 cm away from the semicircular incision. A pipet was inserted through the deep SC space connecting the two incisions acting as a guide wire. Subsequently, a hollow silicone tube featuring an inner diameter larger than the diameter of the device was fed along the guide wire from the semicircular incision. The guide wire was then withdrawn, and the cell encapsulation unit (i.e. the silicone tubing and attached rat islet encapsulation hydrogel) of the device was inserted into the lumen of the hollow silicone tube. The hollow silicone tube was then pulled out through the transverse incision, leaving the device situated within the SC space. Finally, the SC tissue around the tank was sutured using 3-0 polyglactin 910 sutures, then the skin was closed using 3-0 nylon sutures.

For retrieval, a 3 cm incision was made along the tank, and the devices were pulled out after cutting open the surround fibrotic capsule. The subcutaneous tissue was sutured using 3-0 polyglactin 910 sutures, and then the skin incision was closed using 3-0 nylon sutures.

BG monitoring & intraperitoneal glucose tolerance tests (IPGTT). Mouse BG levels were measured by a commercial glucometer (Contour Next EZ, Bayer) with a drop of blood collected from tail vein. For the IPGTT, mice were fasted for 16 h and administered an intraperitoneal injection of 20% glucose solution (2 g of glucose/kg mouse). BG levels were measured at 0, 15, 30, 60, 90, and 120 min (an additional recording at 180 min was made for iBEDv1) following glucose injection.

Ex vivo static GSIS assay. Krebs Ringer Bicarbonate (KRB) buffer was prepared as follows: 2.6 mM CaCl₂·2H₂O_{, 1.2} mM MgSO₄·7H₂O_{, 1.2} mM KH₂PO₄, 4.9 mM KCl, 98.5 mM NaCl, and 25.9 mM NaHCO₃ (all from Sigma-Aldrich), supplemented with 20 mM HEPES (Gibco) and 0.1% BSA (Sigma-Aldrich). The retrieved devices (without the surrounding tissue) were incubated in KRB buffer supplemented with 2.8 mM glucose for 2 h at 37° C., 5% CO₂. Devices were transferred and incubated in KRB buffer supplemented with 2.8 mM, then 16.7 mM glucose for 75 min each. The buffer was collected after each incubation step, and insulin concentration was measured using an ultrasensitive rat insulin ELISA kit (ALPCO).

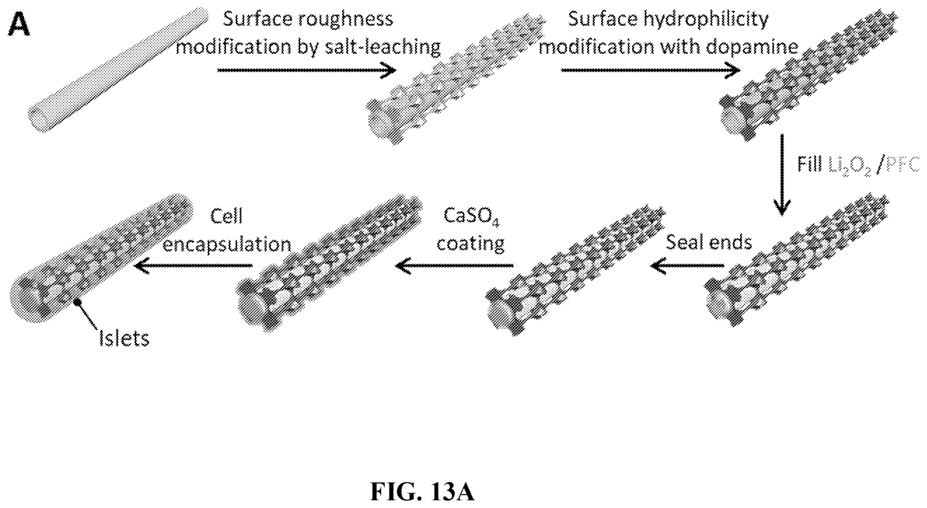

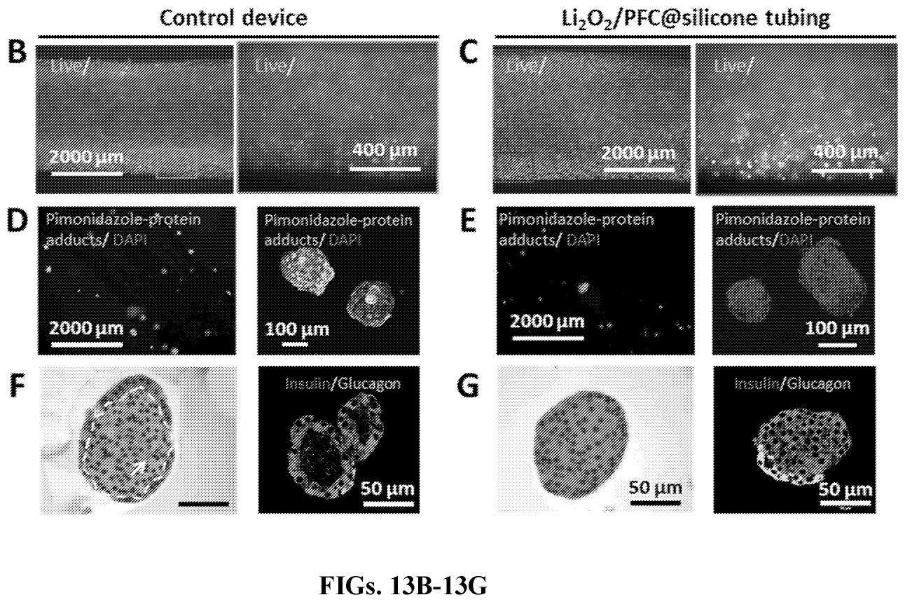

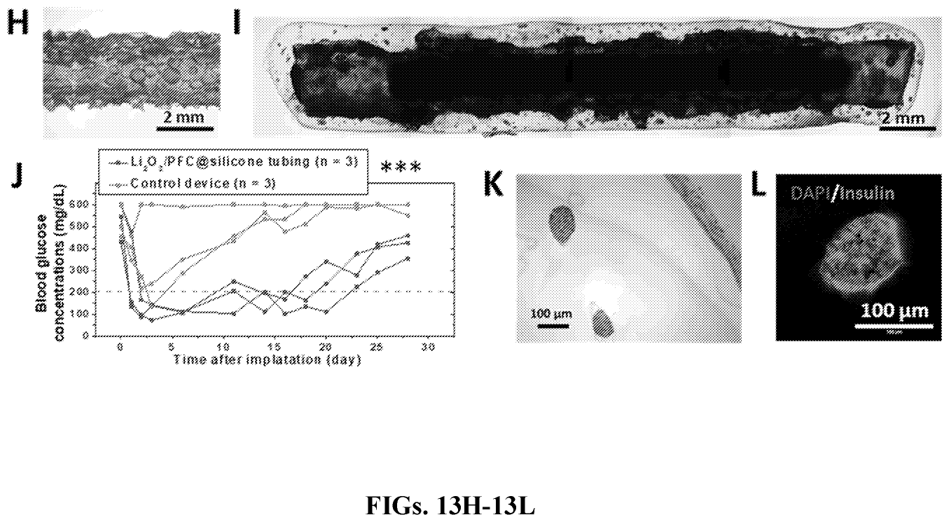

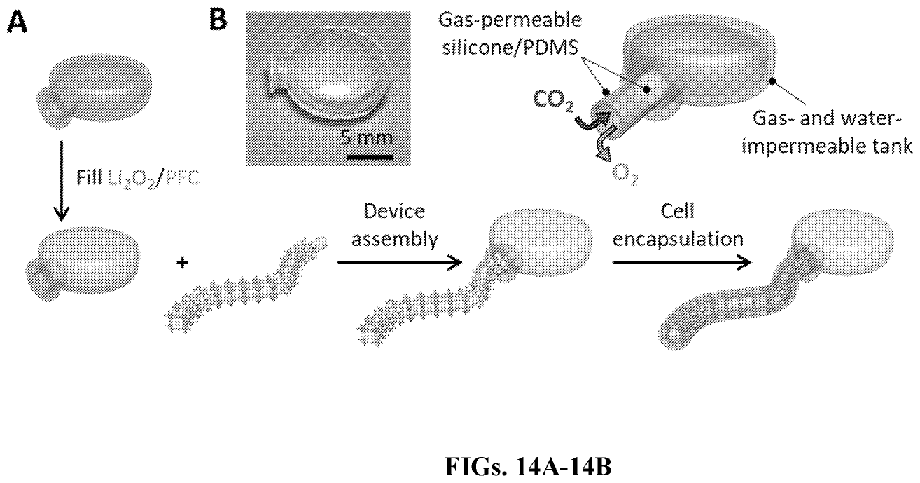

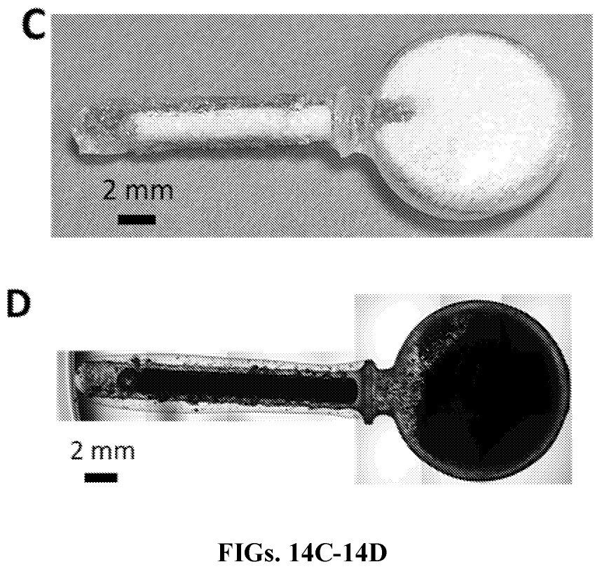

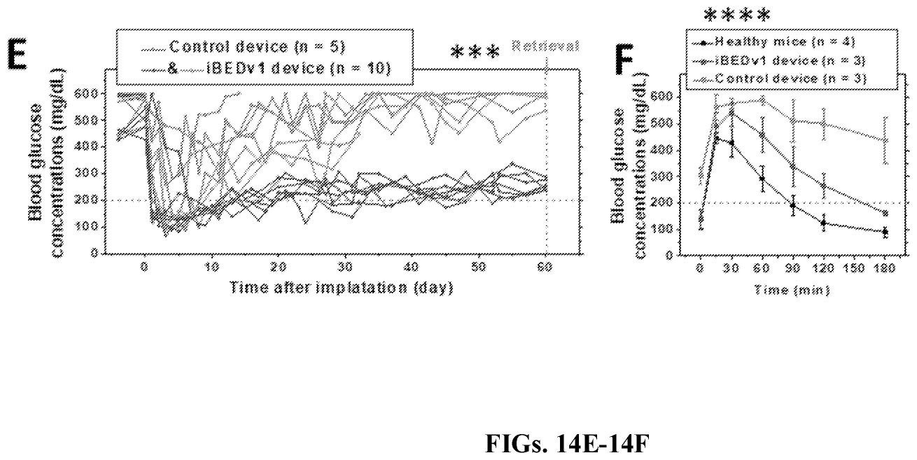

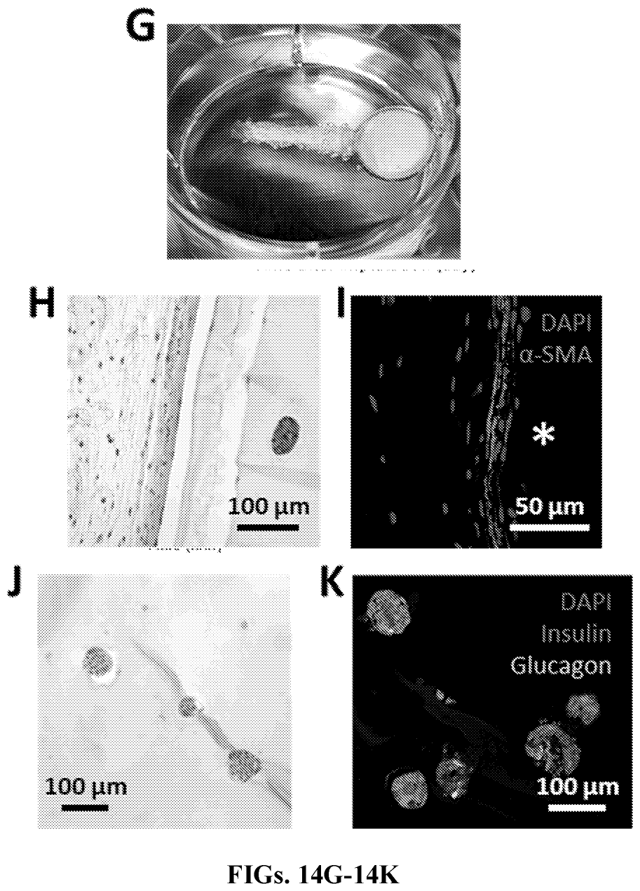

Statistics. Results are expressed as raw data or mean±SD. For random BG measurements (FIG. 13J, FIG. 14E and FIG. 7J), a one-way analysis of covariance (ANCOVA) was performed for measurements between day 2 and the day of device retrieval, where treatment condition (i.e. iBED-treated mice versus control-treated mice) were considered independent variables and time was considered a continuous covariate. Inclusion or omission of the failed devices in the iBED-treated group did not change the significance conclusion for the analysis of data in both FIG. 14E and FIG. 7J. For FIG. 14E and FIG. 7J, the data was reanalyzed via a two-way analysis of variance (ANOVA) followed by a Tukey's post-hoc p-value adjustment, where three treatment groups (iBED-treated mice and control-treated mice) were considered independent variables and time, in this case, was considered a discrete factor to test for differences between treatment groups. This analysis also did not change the significance conclusion for these data. For the IPGTT tests (FIG. 14F and FIG. 7K), data was analyzed via a two-way ANOVA followed by a Tukey's post-hoc p-value adjustment, where treatment (i.e. healthy mice, diabetic mice, control-treated mice, and iBED-treated mice) were considered independent variables and time was considered a discrete factor. For the GSIS test (FIG. 7L), data was analyzed via a one-tailed paired students t-test. Statistical significance was concluded at p<0.05. All statistical analyses were performed in R software.

### Example 1—A CO₂-Regulated O₂ Delivery System for Encapsulated Cells

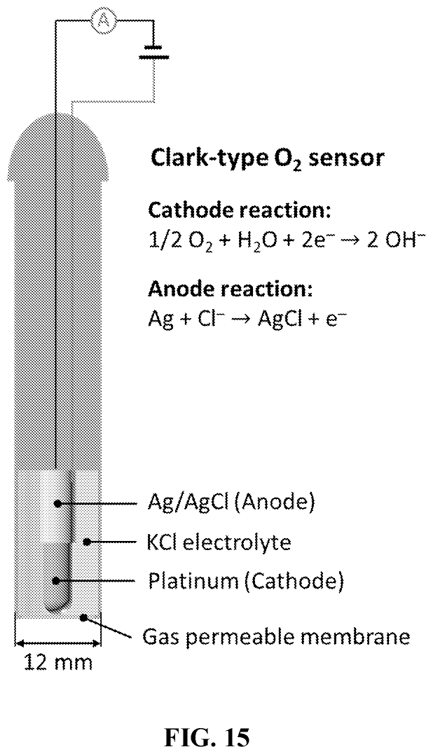

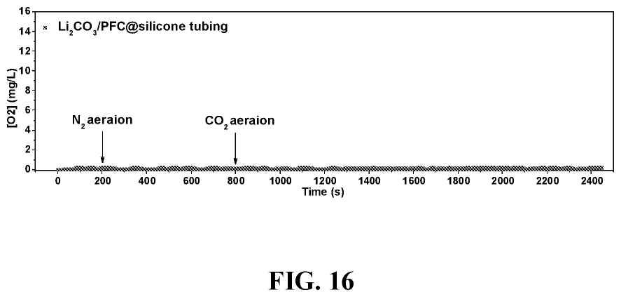

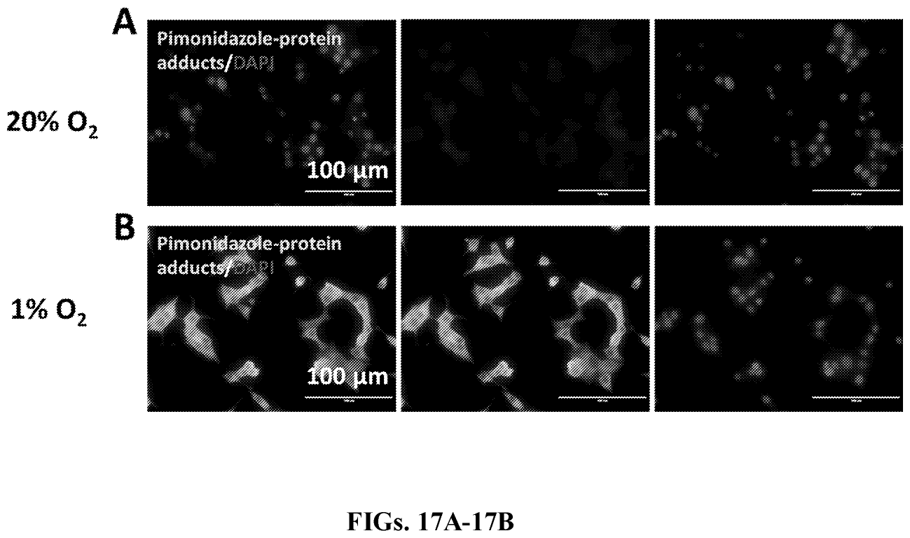

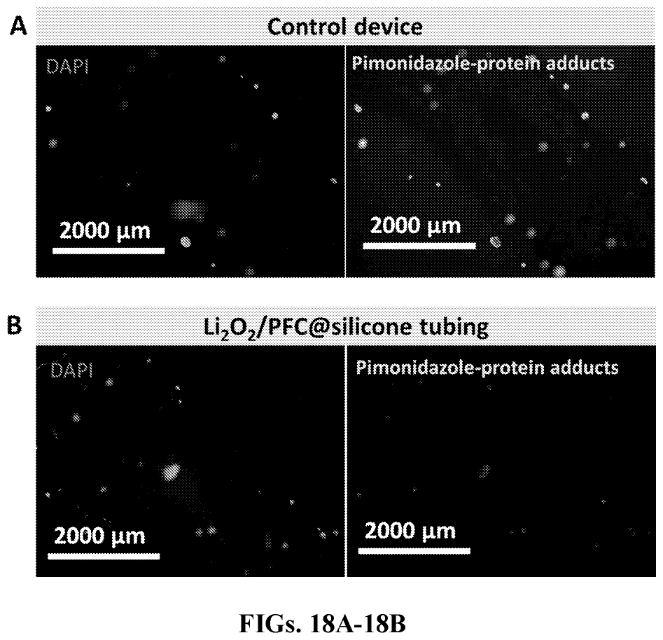

A simple construct was developed to demonstrate the ability of silicone membrane-isolated Li₂O₂ to recycle CO₂ into O₂. Biocompatible (USP Class VI) silicone tubing (2 cm length, i.d.˜1.47 mm, o.d.˜1.96 mm) was filled with 20% (w/w) Li₂O₂ particulates immersed in PFC oil (Li₂O₂/PFC) and sealed at the ends with curable PDMS resin (FIG. 1). Silicone-based materials were desirable due to their high gas (e.g. O₂ and CO₂) permeability (Merkel et al., “Gas Sorption, Diffusion, and Permeation in Poly (Dimethylsiloxane),” J. Polym. Sci., Part B: Polym. Phys. 38:415-434 (2000), which is hereby incorporated by reference in its entirety), good corrosion resistance (Lee et al., “Solvent Compatibility of Poly (Dimethylsiloxane)-based Microfluidic Devices,” Anal. Chem. 75:6544-6554 (2003), which is hereby incorporated by reference in its entirety), and ability to physically isolate the particulate suspension. PFC oil was an optimal medium for Li₂O₂ because of its high O₂ and CO₂ solubility and its water-resistant properties (Riess et al., “Fluorocarbon-based in Vivo Oxygen Transport and Delivery Systems,” Vox Sang. 61:225-239 (1991), which is hereby incorporated by reference in its entirety), as the presence of water may unwantedly accelerate the O₂ generating reaction (Earnshaw, “Chemistry of the Elements.” (1997), which is hereby incorporated by reference in its entirety). In concept, CO₂ from the encapsulated cells and the surrounding tissues diffuses through the silicone tubing and is recycled into O₂ via Li₂O₂, which, in turn, diffuses radially to be consumed by the encapsulated cells. To test this concept, the Li₂O₂/PFC@silicone construct was submerged in an aqueous solution containing sodium sulfite (Na₂SO₃), an O₂ scavenging agent used to maintain negligible dissolved O₂ levels in the solution, and subjected to three aeration phases: (1) a 100% nitrogen gas (N₂) phase, (2) a 100% CO₂ phase, and (3) a second 100% N₂ phase (FIGS. 1C-1F). A lithium carbonate (Li₂CO₃)/PFC formulation, which is inert to CO₂, was used in place of Li₂O₂/PFC as a negative control. O₂ measurements with a Clark electrode (FIG. 15) showed baseline O₂ levels (˜0 mg/L) during the first N₂ aeration phase (FIG. 1E), a rapid increase from bubbles released from the tubing surface during the CO₂ aeration phase (FIG. 1F), and a return to near-zero levels during the second N₂ aeration phase after the purging of bubbles formed in the second phase (FIG. 1G). No O₂ production was observed in control Li₂CO₃/PFC@silicone samples following testing under this aeration regime (FIG. 16). In addition, incubation of the Li₂O₂/PFC@silicone construct in saline for several weeks did not change the solution pH. These findings validated that the silicone-encapsulated Li₂O₂/PFC formulation was CO₂-responsive and securely packed.

### Example 2—Improvement of Cell Survival During In Vitro Hypoxic Incubation and In Vivo

The Li₂O₂/PFC@silicone construct was subsequently adapted to support cell encapsulation and investigated in its ability to improve cell viability during hypoxic incubation in vitro (FIG. 13). Here, the surface of the silicone tubing was modified ensure robust attachment of the cell-laden hydrogel coating (FIG. 13A). Briefly, a layer of macroporous PDMS was attached to the silicone tubing to increase the surface roughness (FIG. 5), and a polydopamine coating was then added to provide hydrophilicity. Alginate (a ˜500 μm layer) was chosen for cell encapsulation due to its ease of crosslinking and biocompatibility (de Vos et al., “Alginate-based Microcapsules for Immunoisolation of Pancreatic Islets,” Biomaterials 27:5603-5617 (2006), which is hereby incorporated by reference in its entirety).

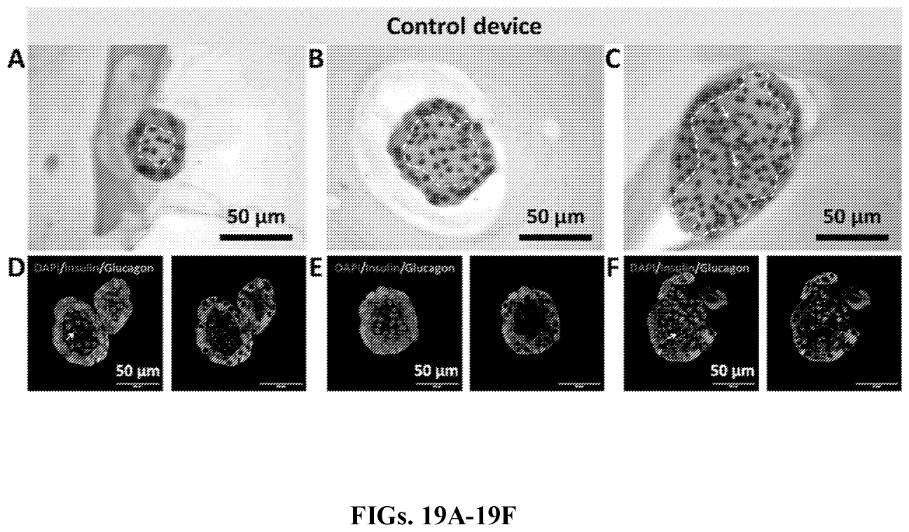

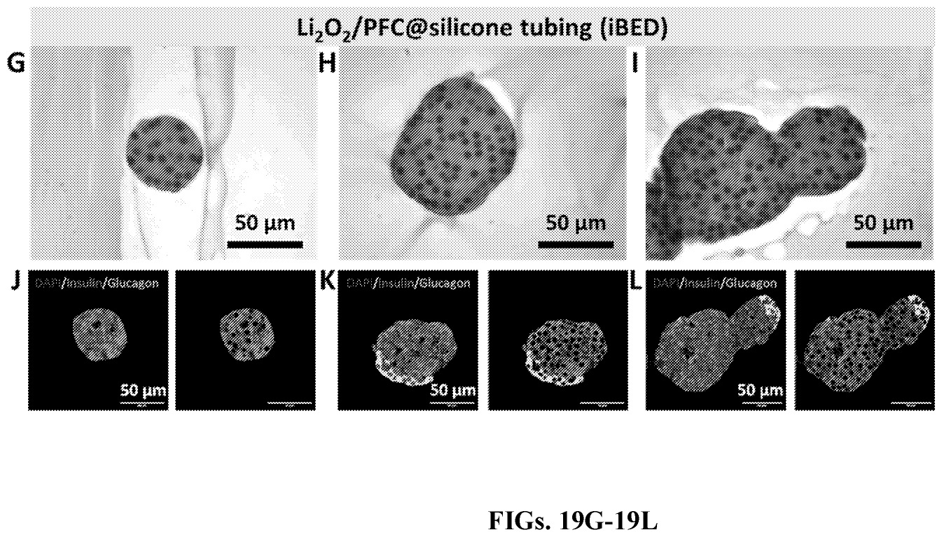

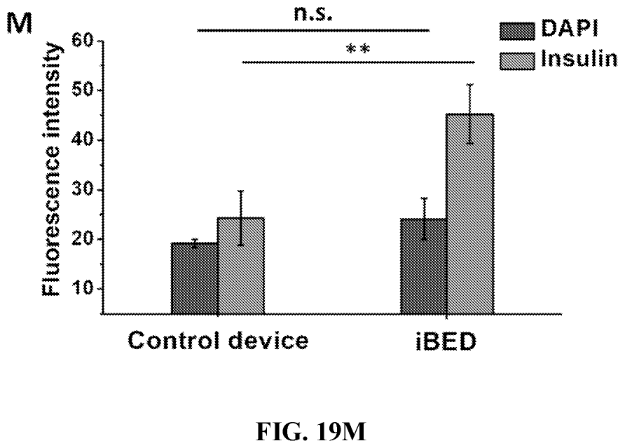

We tested the ability of the Li₂O₂/PFC formulation to enhance cellular O₂ supply by incubating this construct in hypoxic conditions. INS-1 cells (2.5 million cells/mL alginate) were incorporated into Li₂O₂/PFC@silicone constructs and Li₂CO₃/PFC@silicone controls and incubated at 1% O₂, 5% CO₂. Live/dead staining of samples after 24 h of hypoxic incubation revealed that only a thin layer of cells near the hydrogel-buffer interface survived in the controls (FIG. 13B), whereas most cells in the Li₂O₂/PFC@silicone iBED samples were viable (FIG. 13C). The iBED yielded a 2.7-fold improvement in cell viability in comparison to the control device (FIG. 8). This study was also performed with rat islets (500 IEQ within 100 μL of alginate). After 24 h of hypoxic incubation, pimonidazole (FIG. 17) was used to evaluate hypoxia in islets. While a significant accumulation of pimonidazole-protein adducts was observed in the control sample islets (FIG. 13D and FIG. 18A), only minimal amounts were detected in the Li₂O₂/PFC@silicone islets (FIG. 13E and FIG. 18B). Furthermore, hematoxylin and eosin (H&E) and insulin/glucagon immunostaining revealed only 1 to 3 layers of intact and insulin-positive cells in control islets (FIG. 13F and FIGS. 19A-19F), whereas virtually all Li₂O₂/PFC@silicone islets were intact and positive for insulin throughout and glucagon in peripheral cells (FIG. 13G and FIGS. 19G-19L). Additionally, in core cells of nearly all control islets, pyknosis (shrunken and dark nuclei) was detected (FIG. 13F and FIGS. 19 A-19C), and karyorrhexis (fragmented nuclei) were seen in some larger islets (FIG. 13F and FIGS. 19C-19F), signs of apoptosis and necrosis (Moritz et al., “Apoptosis in Hypoxic Human Pancreatic Islets Correlates with HIF-1α Expression,” The FASEB Journal 16:745-747 (2002), which is hereby incorporated by reference in its entirety). Specifically, islets in the control devices showed a 1.86-fold reduction of insulin expression due to the non-insulin secretion cells in the hypoxic core and 1.25-fold reduction in DAPI content because of the shrunken or fragmented nuclei compared to islets in the iBED devices (FIG. 19M). These outcomes indicated that O₂ production from Li₂O₂/PFC-containing constructs improved cell survival and function under hypoxic culture conditions.

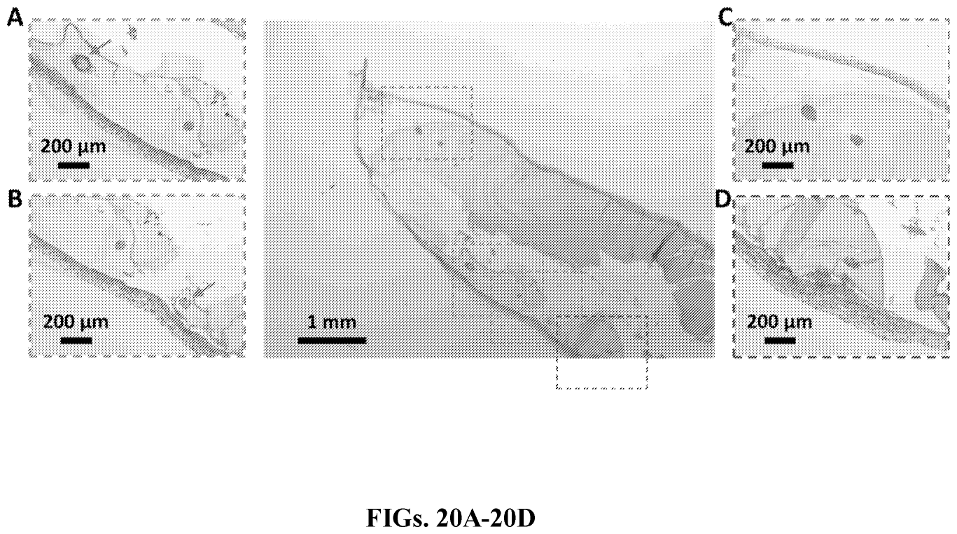

In vivo outcomes of the Li₂O₂/PFC@silicone cell encapsulation construct were explored next. The SC space was selected as the site of implantation for its clinical desirability. Its low pO₂ levels, reported to range from <8-40 mmHg (Carreau, “Why is the Partial Oxygen Pressure of Human Tissues a Crucial Parameter? Small Molecules and Hypoxia,” J. Cell. Mol. Med. 15:1239-1253 (2011) and Najdahmadi et al., “Non-Invasive Monitoring of Oxygen Tension and Oxygen Transport Inside Subcutaneous Devices After H2S Treatment,” Cell Transplant. 29:0963689719893936 (2020), which are hereby incorporated by reference in their entirety), also provided a challenging hypoxic environment to test the capability of the construct. Rat islets (500 IEQ/transplant) in 100 μL of alginate were incorporated in Li₂O₂/PFC@silicone constructs (n=3) and Li₂CO₃/PFC@silicone controls (n=3) and transplanted in the dorsolateral SC space of STZ-induced diabetic C57BL6/J mice (FIGS. 13H-13I). BG lowering was observed in control-treated mice for only 1-3 days following transplantation, whereas hyperglycemia reversal (<200 mg/dL) was sustained for 16-20 days in the group treated with the Li₂O₂/PFC@silicone samples (FIG. 13J). H&E staining of retrieved islets from Li₂O₂/PFC@silicone samples showed several well-defined islets with no observable signs of hypoxia (FIG. 13K), though a few unhealthy islets were found as well (FIGS. 20A-20B). Additionally, immunostaining revealed insulin-positive cells (FIG. 13L), suggestive of maintained islet function.

These studies confirmed that the Li₂O₂/PFC formulation, regulated by physiological CO₂, could mitigate hypoxia in cell encapsulation devices, providing a foundational proof-of-concept of the inverse-breathing O₂ generating system.

### Example 3—iBED Prototype Reverses Hyperglycemia in Mice

Encouraging results from the proof-of-concept studies prompted the design of a prototype with the intention of prolonging the duration of O₂ supply (FIG. 14). A first-generation iBED prototype (iBEDv1) was developed, featuring a 3-dimensional (3D)-printed terminal Li₂O₂/PFC reserve tank to increase the formulation storage capacity (FIG. 14A). A dental resin was used for the printing of the terminal tank because it is both biocompatible and impermeable to gas, which prevented O₂ leakage (FIG. 14B and FIG. 6). Briefly, iBEDv1 was fabricated by filling the terminal tank with 20% (w/w) Li₂O₂/PFC and attaching it to the Li₂O₂/PFC@silicone construct (1.5 cm; fabricated as previously described) at the tank neck via a gas-permeable PDMS adapter (FIGS. 14A-14D). The terminal reserve tank increased the Li₂O₂/PFC storage capacity, and likewise theoretical total O₂ output, 8-fold, and featured a deliberately low surface area of gas exchange at the tank neck and was therefore hypothesized to significantly extend the duration of O₂ supply.

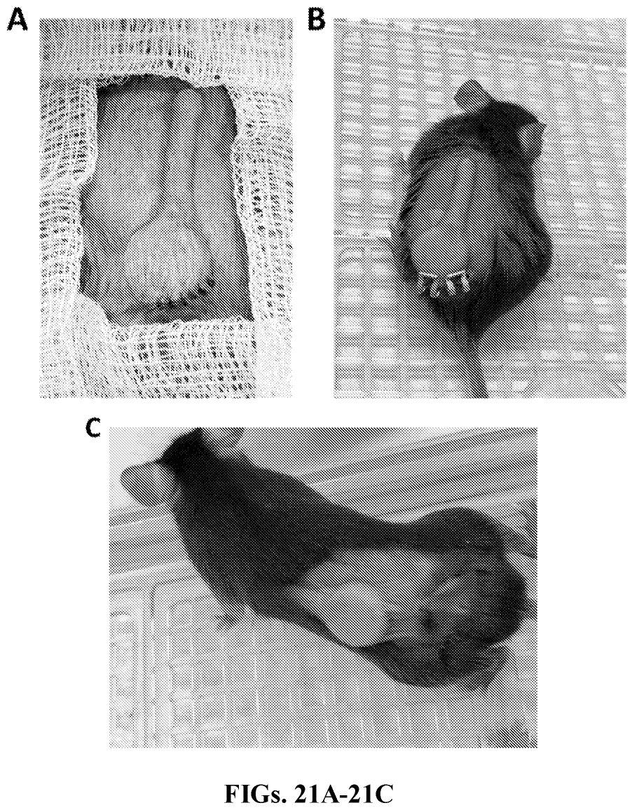

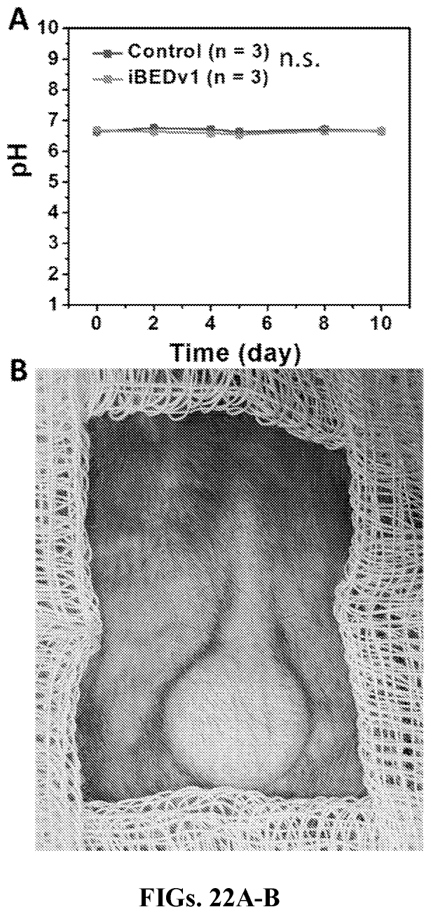

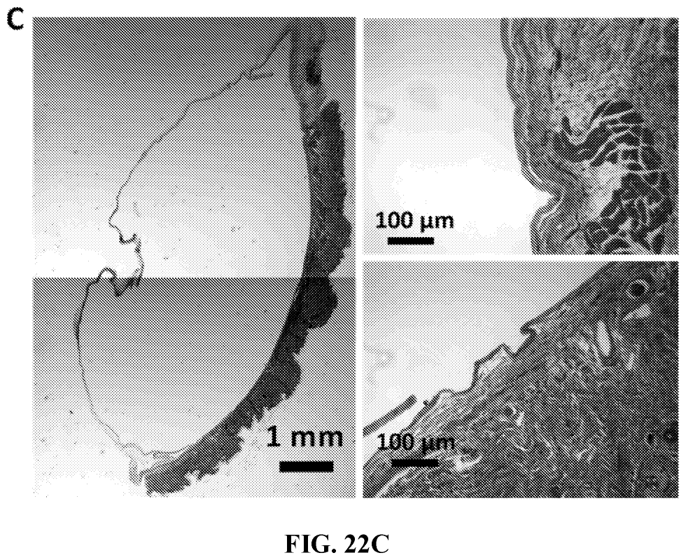

Rat islets (500 IEQ/transplant) within 80 μL of alginate were incorporated in iBEDv1 prototypes (n=10) and controls which did not include the Li₂O₂/PFC formulation (n=5). The devices were transplanted in the dorsolateral SC space of STZ-induced diabetic C57BL6/J mice (FIG. 21 and FIG. 22). Control-treated mice exhibited lowered random BG levels for 1-10 d before returning to a hyperglycemic state, whereas 7 of 10 iBEDv1-treated mice exhibited lowered BG levels over 60 d (FIG. 14E). While normoglycemia (<200 mg/dL) was restored for approximately 15 d in 7 of 10 iBEDv1-treated samples, the BG rose steadily to a range between 200-300 mg/dL following this initial period. This coincided with the time frame of normal BG restoration for the simple Li₂O₂/PFC@silicone construct (FIG. 13J), suggesting a possible drop in efficacy once the particulates within the lumen of the silicone tubing were consumed.

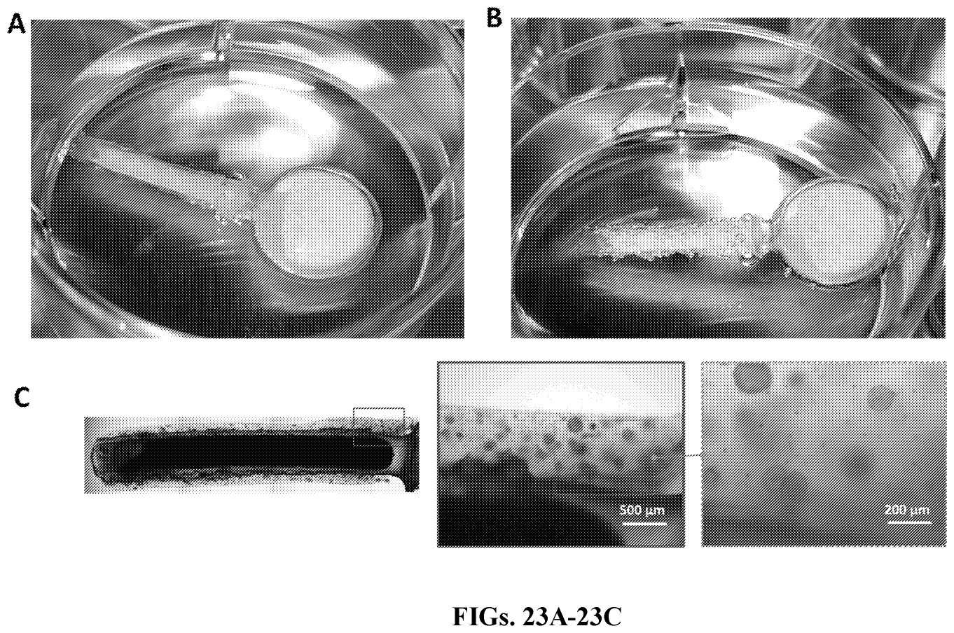

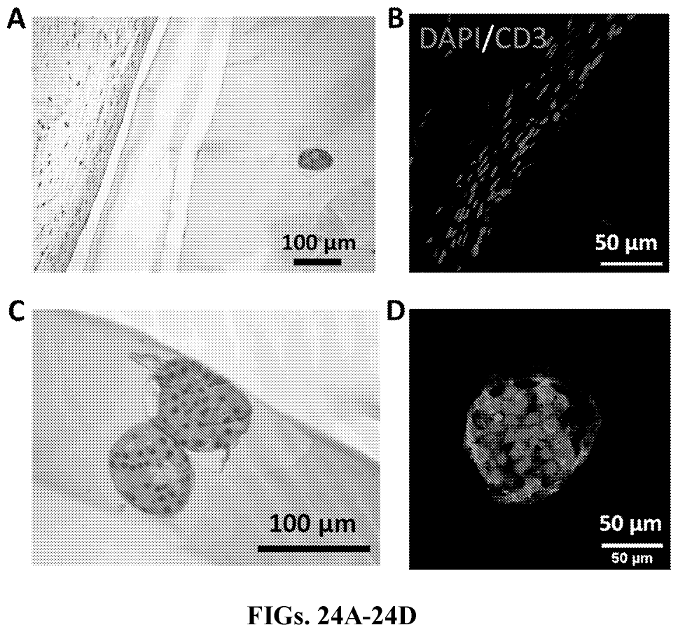

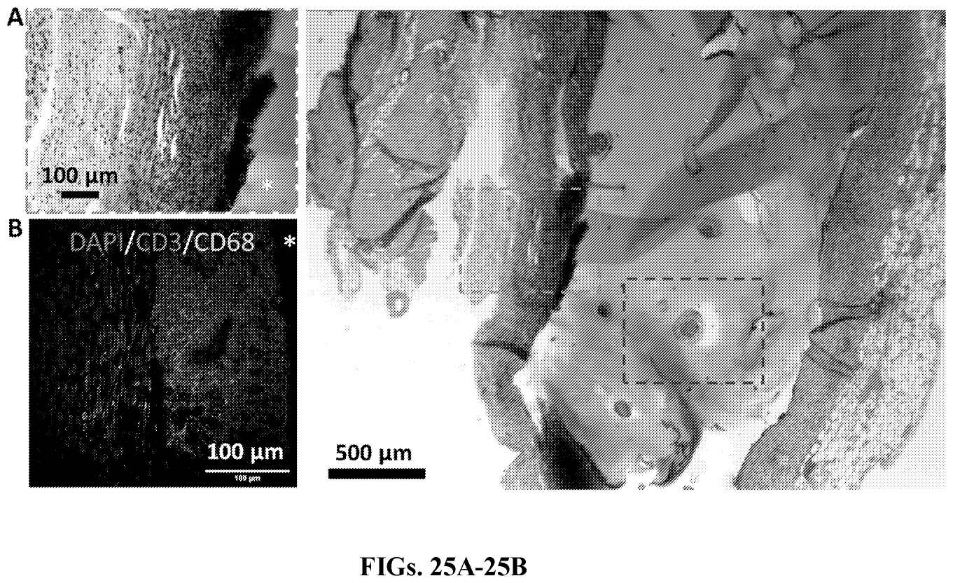

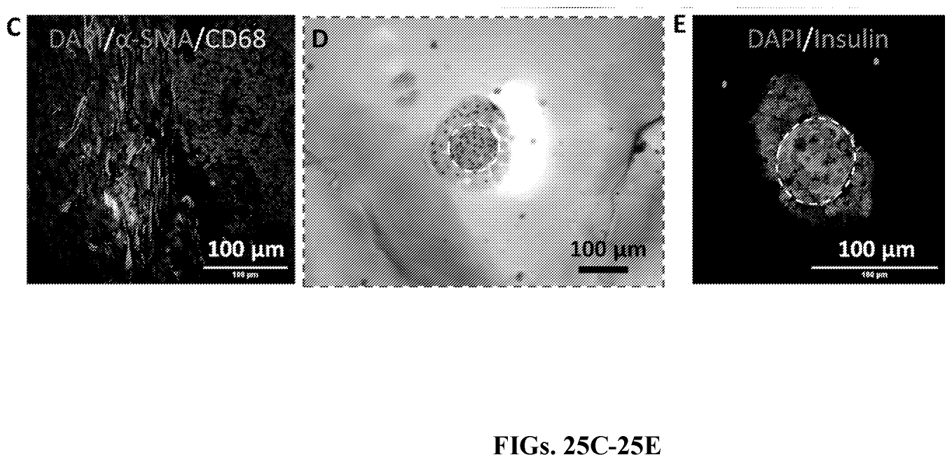

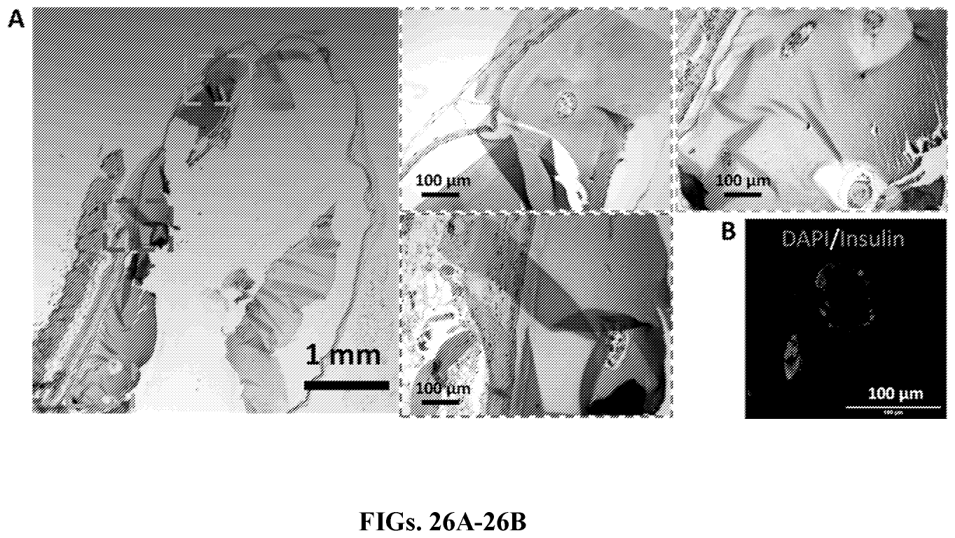

An intraperitoneal glucose tolerance test (IPGTT) was administered at 58 d to three iBEDv1-treated subjects, each of which exhibited moderate glycemia at this stage (FIG. 14F). The BG of the treated group returned to normoglycemia after 120-180 min (slightly delayed in comparison to healthy control mice), whereas the BG of the control device-treated mice did not fall below 400 mg/dL during this period. Following retrieval, the hydrogel was stripped from one device for imaging and staining and the tank/silicone tubing assembly was incubated in saline buffer at 5% CO₂ for 3 h. Bubbles were observed at the tubing surface, suggesting that O₂ generation was still active at 60 d (FIG. 14G). Interestingly, bubbles first appeared on the tubing closer to the tank and gradually formed down the lumen over time, indicating the possible presence of an O₂ gradient in z direction of the tubing lumen (FIG. 23). According to H&E staining, a modest deposition of fibrotic tissue was found adjacent to the hydrogel (FIG. 14H). The fibrotic tissue showed no signs of inflammation, myofibroblasts were identified by alpha smooth muscle actin (α-SMA) staining (FIG. 14I), and only rare and sporadic T cells were found in CD3 staining (FIG. 24B). H&E-stained iBEDv1 islets in animals with moderately controlled BG were observed with preserved morphology (FIG. 14J), and immunostaining revealed several insulin- and glucagon-positive cell clusters (FIG. 14K), though a few unhealthy islets were found far away from the tank, possibly due to limited O₂ transport to this region (FIGS. 24C-24D). Some islets in a retrieved iBEDv1 from a mouse with elevated BG levels (FIG. 25) showed necrosis in the mantle of the islet, rather than the core, suggesting that the observed cell death might not be due to hypoxia (FIGS. 25D-25E). Indeed, higher amounts of T cells and macrophages were found in the fibrotic tissue surrounding failed iBEDv1 samples (FIGS. 25A-25C) in comparison to successful ones. Control islets were largely fragmented, and several were totally necrosed (FIG. 26).

The promising results from this study further confirmed the benefit of this CO₂-responsive O₂ delivery system to encapsulated islets and suggested that the duration of O₂ supply could be extended by attaching an additional Li₂O₂/PFC reservoir in connection with the device.

### Example 4—Model-Aided Design Optimization

Computational modeling was used to estimate the impact of design modifications on islet oxygenation (FIG. 11). The iBEDv1 prototype featured a silicone tubing filled with the Li₂O₂/PFC formulation. However, the Li₂O₂ content within the lumen was only sufficient to produce O₂ to support the encapsulated rat islets for ˜1 week. After the O₂ content within the lumen was consumed, it was expected that slow O₂ diffusion in the formulation would retard transport between the tank and cells. The second-generation device design (iBEDv2) thus considered the replacement of the Li₂O₂/PFC formulation in the lumen with air (by simply not filling it), hypothesizing that this would significantly improve overall O₂ delivery, as diffusion in air is roughly 10,000-fold faster than in PFC (O'Brien et al., “Diffusion Coefficients of Respiratory Gases in a Perfluorocarbon Liquid,” Science: 153-155 (1982) and Chapman et al., “The Mathematical Theory of Non-Uniform Gases: An Account of the Kinetic Theory of Viscosity, Thermal Conduction and Diffusion in Gases,” Cambridge University Press (1990), which are hereby incorporated by reference in their entirety). Expanding the advantage of fast gas-phase O₂ diffusion, a hollow channel through-port from the lumen into the tank was incorporated to accelerate gas exchange. Decreasing the silicone tubing thickness to reduce the diffusive resistance of O₂ over this silicone barrier and increasing its surface area to improve O₂ distribution throughout the hydrogel phase was considered. The third-generation design (iBEDv3) featured a quadruple-helix of thinner hollow silicone tubing (i.d.˜0.34 mm, o.d.˜0.64 mm). O₂ transport was simulated in a finite element model to compare the relative efficacy of these designs in comparison to a control device without O₂ supply (FIGS. 11A-11B, FIG. 9, and FIG. 10).

In general, the iBEDv2 and iBEDv3 designs performed more desirably than the control device and iBEDv1. Cross-sectional surface plots showed a uniformly low pO₂ distribution in the control device, as expected due to the lack of an exogenous O₂ supply system (FIG. 11C). Interestingly, a steep pO₂ gradient was visualized along the z direction in iBEDv1, with pO₂ levels resembling that of the control levels by the midpoint of the device. This indicated that the formulation did indeed significantly restrict O₂ transfer to regions far away from the tank. The iBEDv2 and iBEDv3 designs, however, displayed high and uniform oxygenation throughout, attributed to the vastly superior gas-phase diffusivity within the tubing lumens. Distributions of pO₂ on the islet surfaces further show that only islets near the tank were well oxygenated in iBEDv1, whereas islet oxygenation was independent of tank distance in iBEDv2 and iBEDv3 (FIG. 11D and FIG. 12). The volume-average pO₂ of each islet was calculated, showing that oxygenation was strongly inversely correlated with islet diameter (FIG. 11E), a result of the high O₂ consumption rate of rat islets (Avgoustiniatos et al., “Measurements of the Effective Diffusion Coefficient of Oxygen in Pancreatic Islets,” Ind. Eng. Chem. Res. 46:6157-6163 (2007), which is hereby incorporated by reference in its entirety). Nonetheless, volume-average islet pO₂ fell below 8 mmHg (the threshold necessary for insulin secretion) (Dionne et al., “Effect of Hypoxia on Insulin Secretion by Isolated Rat and Canine Islets of Langerhans,” Diabetes 42:12-21 (1993), which is hereby incorporated by reference in its entirety), in a large fraction of control and iBEDv1 islets, whereas even the largest iBEDv2 and iBEDv3 islets were above this threshold. Organizing volume-average islet pO₂ by frequency showed that a majority of islets in the control or iBEDv1 were poorly oxygenated, whereas only a small fraction of poorly oxygenated islets was observed in iBEDv2, and even smaller fraction in iBEDv3 (FIG. 11F). The low frequency of well oxygenated iBEDv1 islets can be attributed to the fact that those near the tank received substantial O₂ whereas those further away did not. The slight advantage of iBEDv3 in comparison to iBEDv2 may be due to the increased surface area and decreased thickness of the silicone tubing, though the slightly smaller alginate diameter may contribute some effect as well. The comparison between each design may be summarized by comparing the pO₂ measured along a centerline of the device (FIG. 11G and FIG. 4). iBEDv1 centerline pO₂ was higher than iBEDv2 or iBEDv3 levels for ˜20% of the device length, but inferior in the remaining ˜80%, and virtually no better than the control in 50%. Additionally, centerline pO₂ was slightly higher in iBEDv3 than in iBEDv2 throughout. Collectively, these results indicated the combined advantages of O₂ supply by the inverse-breathing system and rapid gas-phase O₂ diffusion in the tubing lumens.

Model results provided several critical insights. Of primary concern was the question of whether the magnitude of O₂ generation would contribute a notable benefit. Simulated islet oxygenation showed a substantial improvement in iBED islets in comparison to control levels for all designs. Results also revealed the limitations of filling the silicone tubing lumen with the Li₂O₂/PFC formulation and indicated that the simple adjustment of maintaining a gas phase therein would improve uniformity of O₂ delivery. Finally, the model suggested that further improvements could be achieved by using thinner silicone tubing in a high surface area configuration. In effect, modeling herein enabled the expedient identification of an optimized design.

### Example 5-02 Distribution Mapping Using an Electron Paramagnetic Resonance Imager

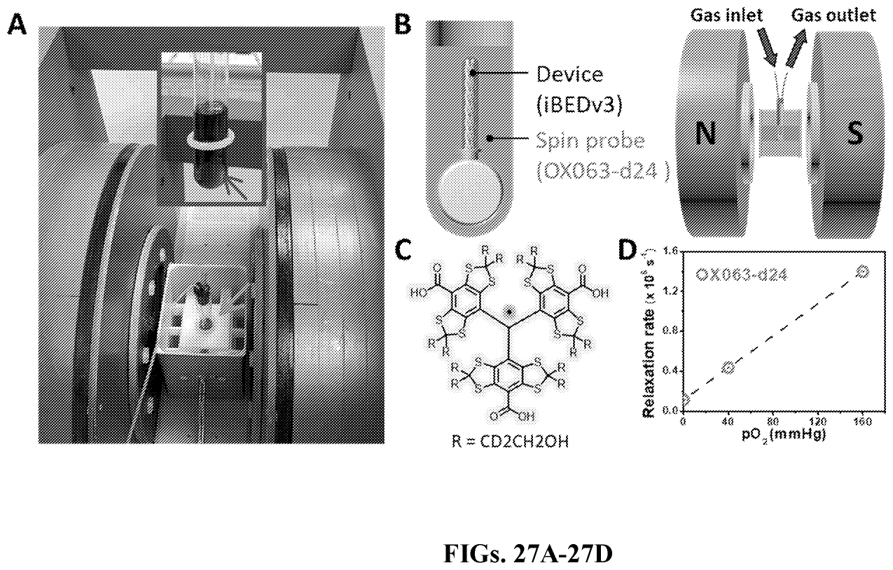

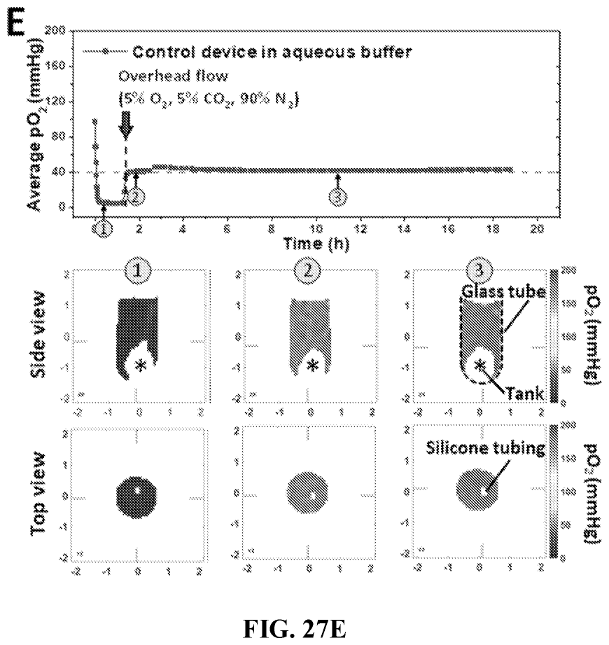

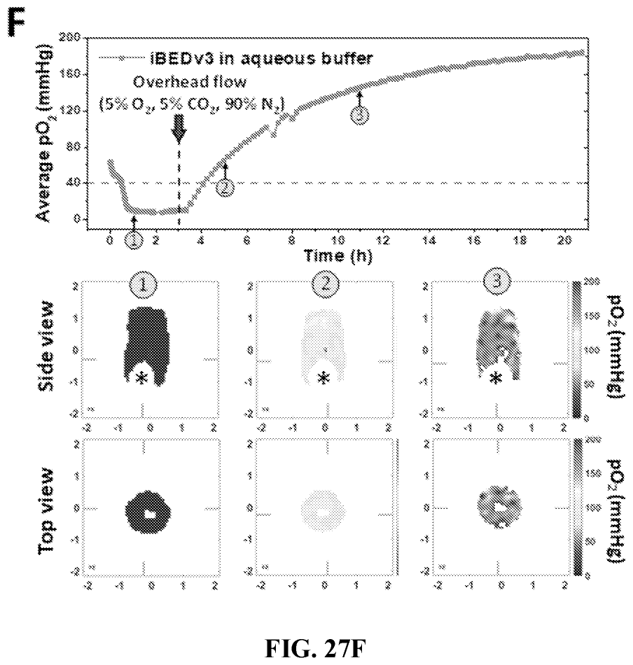

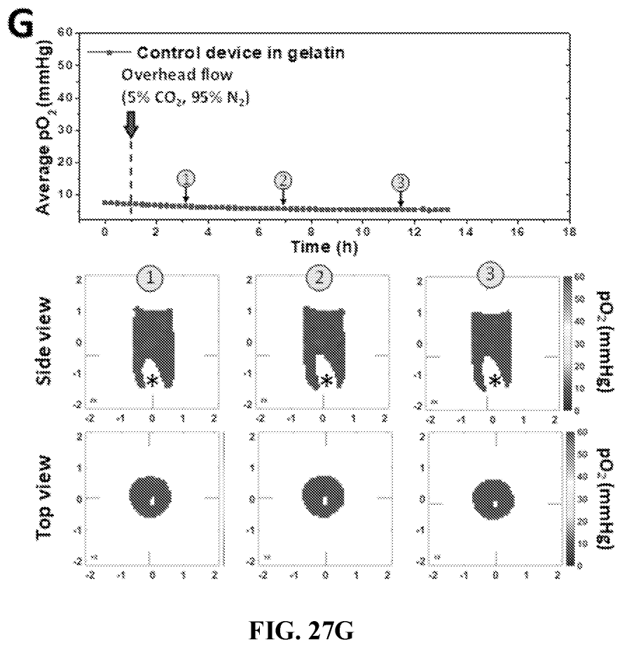

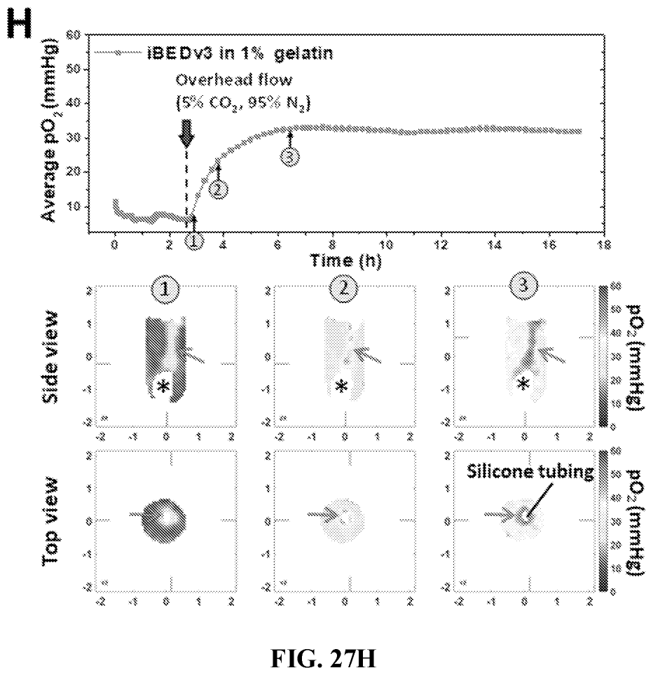

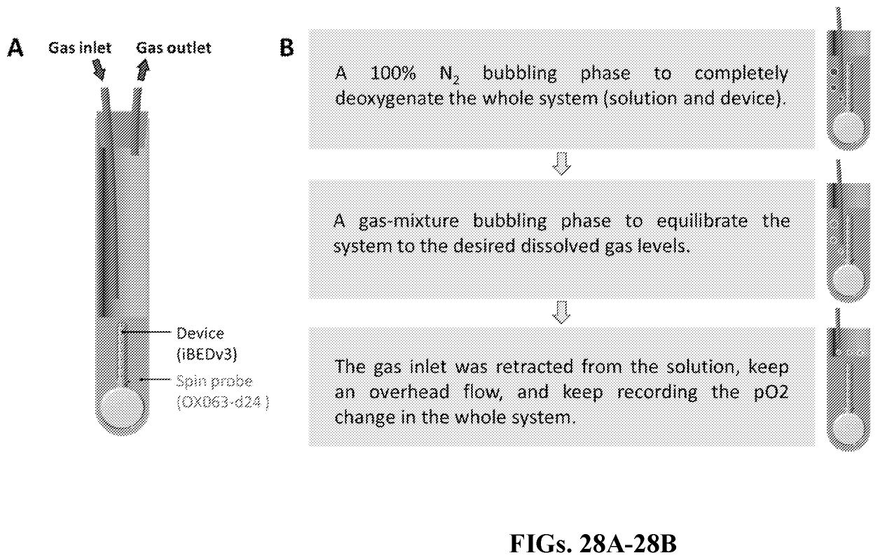

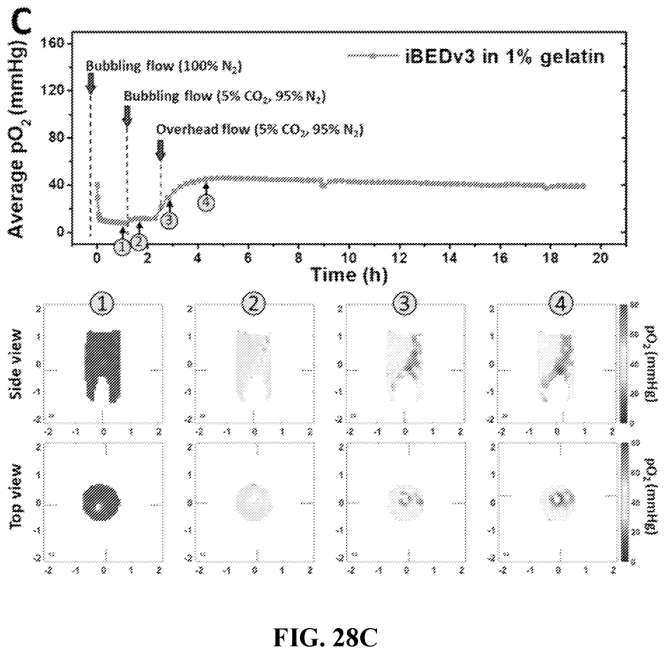

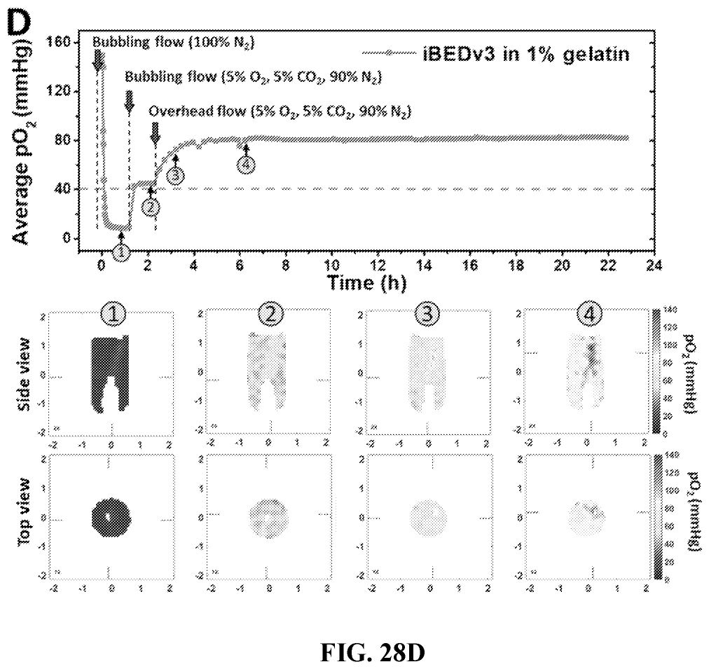

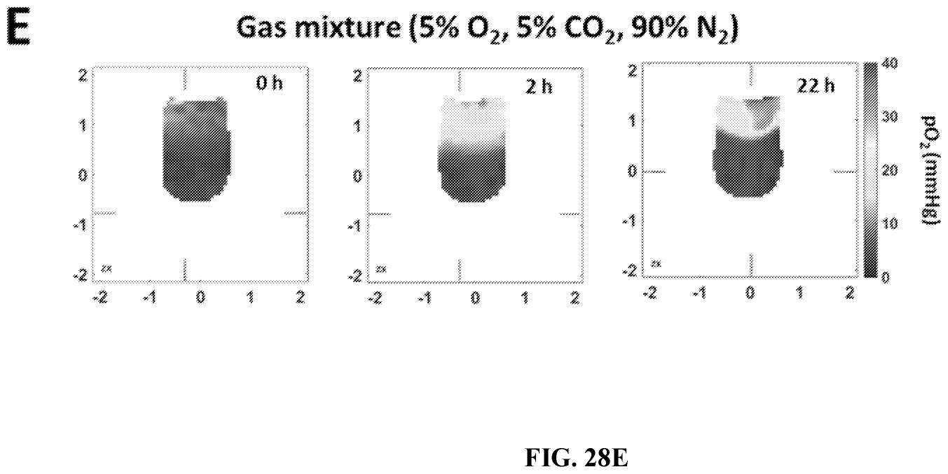

Real-time 3D pO₂ distribution mapping was performed to measure CO₂-regulated O₂ release in the iBEDv3 (FIG. 27 and FIG. 28). These measurements were acquired using pulse electron paramagnetic resonance (EPR) O₂ imaging (Kotecha et al., “Noninvasive Absolute Electron Paramagnetic Resonance Oxygen Imaging for the Assessment of Tissue Graft Oxygenation,” Tissue Eng. Part C Methods 24:14-19 (2018); Epel et al., “In Vivo Preclinical Cancer and Tissue Engineering Applications of Absolute Oxygen Imaging Using Pulse EPR,” J. Magn. Reson. 280:149-157 (2017); and Epel and Halpern, “In Vivo Po2 Imaging of Tumors: Oxymetry with Very Low-Frequency Electron Paramagnetic Resonance,” Methods Enzymol (Elsevier), Vol. 564:501-527 (2015), which are hereby incorporated by reference in their entirety) via a 25 mT preclinical O₂ imager, the JIVA-25 instrument (02M Technologies, LLC, Chicago, IL) (FIGS. 27A-27B). Briefly, devices were submerged in 3 mL of solution in a sealed glass test tube with a water-soluble trityl radical molecular probe (1 mM OX063-d24) (Kuzhelev et al., “Room-temperature Electron Spin Relaxation of Triarylmethyl Radicals at the X- and Q-bands,” J Phys. Chem. B 119:13630-13640 (2015), which is hereby incorporated by reference in its entirety) (FIG. 27C). O₂-sensitive electron spin-lattice relaxation rates (R₁) of the trityl radical in phosphate-buffered saline (at 1 mM) were measured by JIVA-25 and converted into pO₂ according to a calibration curve (FIG. 27D). A gas inlet and a gas outlet were introduced to the sealed glass test tube (FIG. 28A) to subject the devices to an aeration regime (FIG. 28B): (1) a 100% N₂ bubbling phase to deoxygenate the system, (2) a gas-mixture bubbling phase to equilibrate the system to the desired dissolved gas levels, and (3) an overhead flow phase, whereby the gas inlet was retracted from the liquid to the space above the sample and the gas mixture was circulated over the solution, to maintain exposure to the supplied gas mixture after equilibration while reducing perturbance of the fluid.

This procedure was first performed with a control device and an iBEDv3 in an aqueous solution, using a gas mixture of 5% CO₂, 5% O₂, 90% N₂. Average pO₂ measurements of the whole system (i.e. solution and device) and distribution mapping showed that the control sample rose from ˜5 mmHg after deoxygenation to near equilibrium levels (pO₂˜40 mmHg) for the remainder of the experiment (FIG. 27E). On the other hand, the pO₂ in the iBEDv3 sample exceeded equilibrium pO₂ rapidly during the overhead flow phase and continued rising even at 20 h to ˜180 mmHg (FIG. 27F), confirming that O₂ production in iBEDv3 was indeed responsive to physiological CO₂ levels (i.e. pCO₂˜40 mmHg).

We then repeated this study with the devices submerged in 1% gelatin, which is more viscous than water, and was thus expected to reduce fluid mixing and therefore resolve pO₂ gradients in the system. Here, an O₂-free gas mixture of 5% CO₂, 95% N₂ was used. In the control sample, average pO₂ levels trended towards ˜0 mmHg after deoxygenation over the duration of the experiment, and pO₂ distributions were uniform and low (FIG. 27G). In the iBEDv3 sample, average pO₂ reached a steady state of 30-40 mmHg after ˜2 h of overhead flow (FIG. 27H and FIG. 28C). A clear pO₂ gradient was distinguishable at all time points, where pO₂ values were highest near the silicone tubing and decreased with distance. At steady state (>2 h), the pO₂ values near the silicone tubing were between 60-80 mmHg (FIG. 27H and FIG. 28C); notably, they also appeared to be constant with distance from the tank, suggesting uniform oxygenation throughout the silicone tubing lumens, corroborating model predictions. Repeating iBEDv3 measurements in 1% gelatin with a gas mixture of 5% CO₂, 5% O₂, 90% N₂ showed an average steady state pO₂ of ˜80 mmHg, with steady state pO₂ levels near the silicone tubing at ˜140 mmHg (FIG. 28D). It was suspected that, in studies with 1% gelatin, the rise in average sample pO₂ did not continue for as long as in studies with water because of poor CO₂ penetration from overhead flow; this was supported by measurements showing almost no O₂ penetration beyond ˜0.5 cm in 1% gelatin at ˜22 h during 5% O₂ overhead flow (FIG. 28E). Nonetheless, these pO₂ distribution studies clearly indicated robust responsiveness to physiological CO₂ levels, and thorough O₂ transport along the silicone tubing of the iBEDv3, supporting its capability to enhance oxygenation in vivo.

### Example 6—iBEDv3 Design Enables 3-Month Diabetes Correction in Mice Via Subcutaneous Implantation

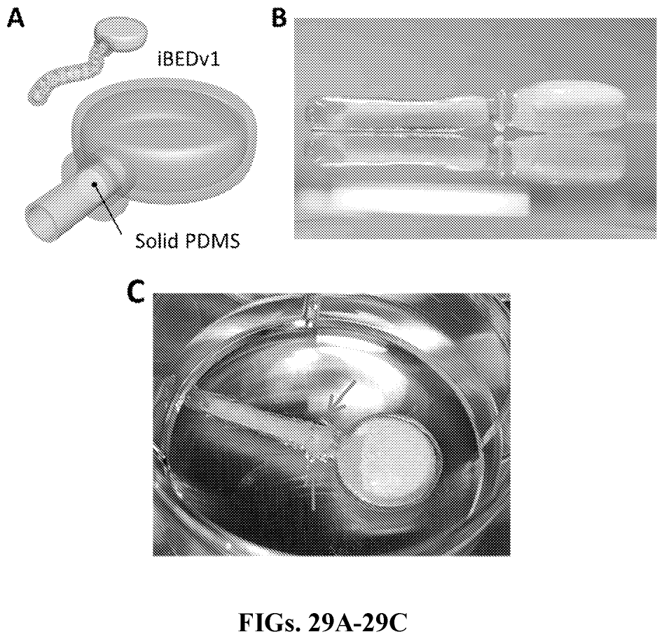

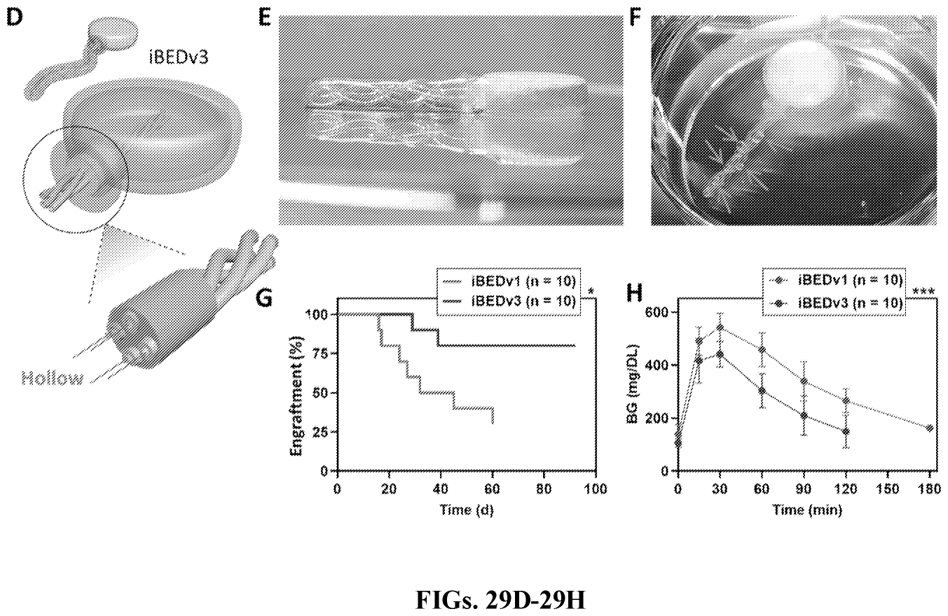

Following O₂ mapping, the iBEDv3 was studied in vivo (FIG. 7). The simulation-guided design improvements and additional modifications were implemented in iBEDv3. Two hollow silicone tubes (i.d.˜0.34 mm, o.d.˜0.64 mm) were twisted, folded at the middle, and then sealed at the ends, creating a four-thread twisted structure resembling the simulated quadruple helix (FIG. 7A). This twisted structure was then fit through a larger, short silicone hollow cylinder (length ˜4 mm, i.d.˜1.47 mm), and fixed into place by filling the larger silicone tubing with PDMS resin and curing. This reduced O₂ and CO₂ diffusive resistance by allowing gas-phase transport through the adapter in the twisted tubing lumens (FIG. 7B-7C), instead of through the solid-phase PDMS as in iBEDv1 and iBEDv2 (FIG. 29A). A liquid perfusion study was performed, confirming that the lumens of the tubing remained hollow after twisting, signaling that gas-phase O₂ transport would not be interrupted (FIGS. 7D-7E). Thereafter, the adapter was affixed to the terminal tank via PDMS resin (FIG. 7F). Rapid formation of bubbles at the tubing surface following a manual injection of air illustrate the high gas permeability of the silicone tubing (FIG. 7G). Finally, islet encapsulation in alginate was performed, yielding the completed device (FIGS. 7H-7I). Note, silicone surface roughness modification was not needed, as hydrogel attachment to the twisted structure was sufficiently robust, consistent with previous findings (An et al., “Designing a Retrievable and Scalable Cell Encapsulation Device for Potential Treatment of Type 1 Diabetes,” Proc. Natl. Acad. Sci. U.S.A. 115, E263-E272 (2018) and Ernst et al., “Interconnected Toroidal Hydrogels for Islet Encapsulation,” Adv. Healthc. Mater. 8, 1900423 (2019), which are hereby incorporated by reference in their entirety).

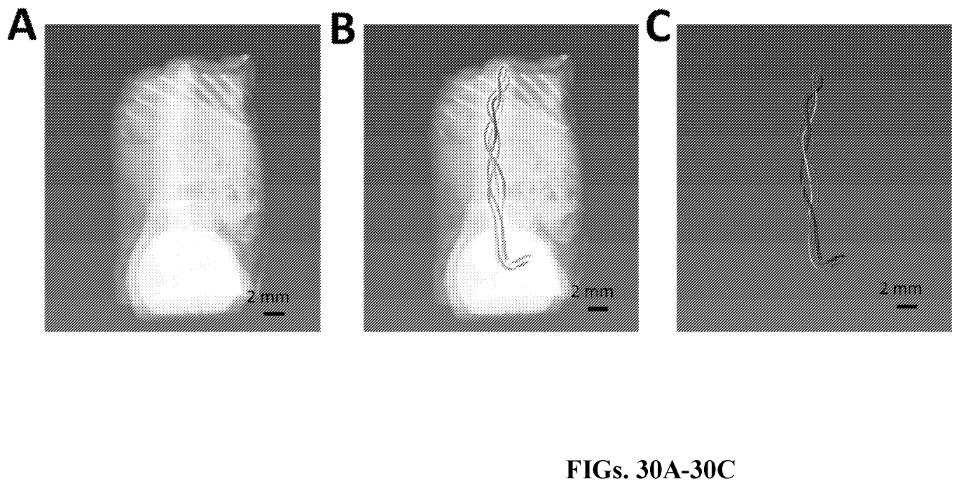

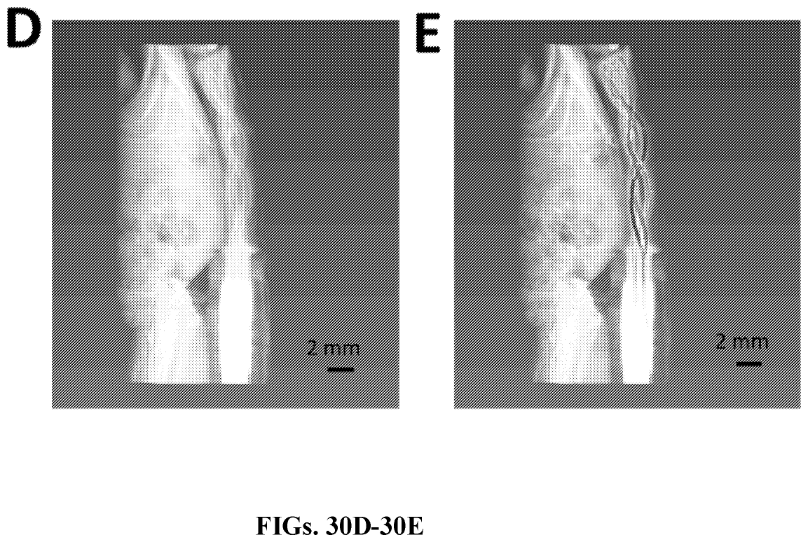

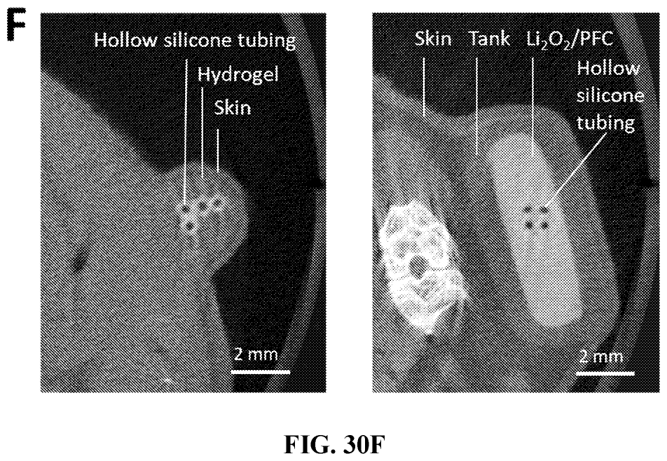

iBEDv3 devices containing Li₂O₂/PFC (n=10) or Li₂CO₃/PFC (controls; n=7), both encapsulating 500 IEQ of rat islets (within 60 μL alginate), were transplanted in the dorsolateral SC space of STZ-induced diabetic C57BL6/J mice. Normoglycemia was achieved in 8 out of 10 iBEDv3-treated mice for 92 d, whereas all control subjects reverted to hyperglycemia quickly after transplantation (FIG. 7J). The engraftment percent of the iBEDv3 was maintained at 80% over 3 months, which was significantly better than the that of the iBEDv1 with the engraftment dropping to 30% at 2 months (FIG. 29G). BG levels in the iBEDv3-treated animals returned to hyperglycemic levels after device retrieval, indicating that the device was responsible for the observed BG regulation. In an IPGTT at 90 d, iBEDv3-treated animals showed lowered BG levels after 90 min, similar to the healthy control mice, whereas control-treated mice did not exhibit lowered BG levels similar to the diabetic control mice (FIG. 7K). Again, the iBEDv3 showed significantly better function than the iBEDv1, showing a faster glucose clearance during the IPGTT (FIG. 29H). In addition, iBEDv3 devices retrieved at 92 d were glucose responsive (stimulation index: 5.6±1.3) in a static glucose-stimulated insulin secretion (GSIS) assay (FIG. 7L). Micro-computed tomography (Micro-CT) imaging of a device implanted in a mouse showed that the hollow lumen structure was maintained (FIG. 7M and FIG. 30).

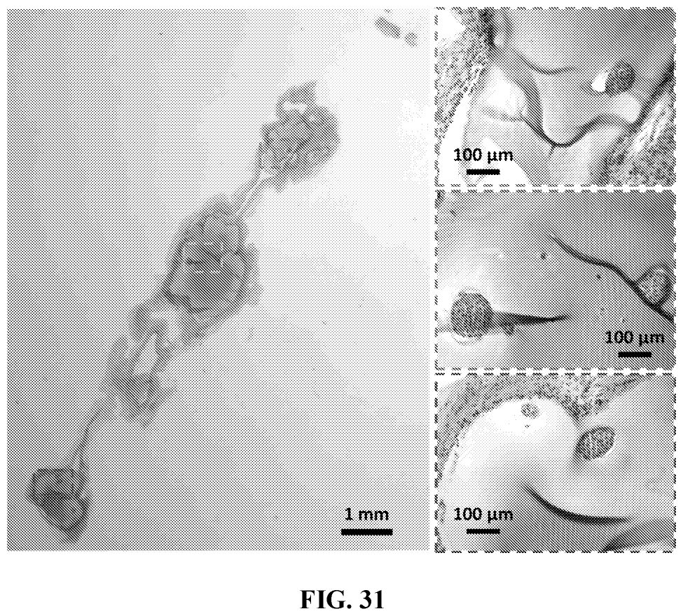

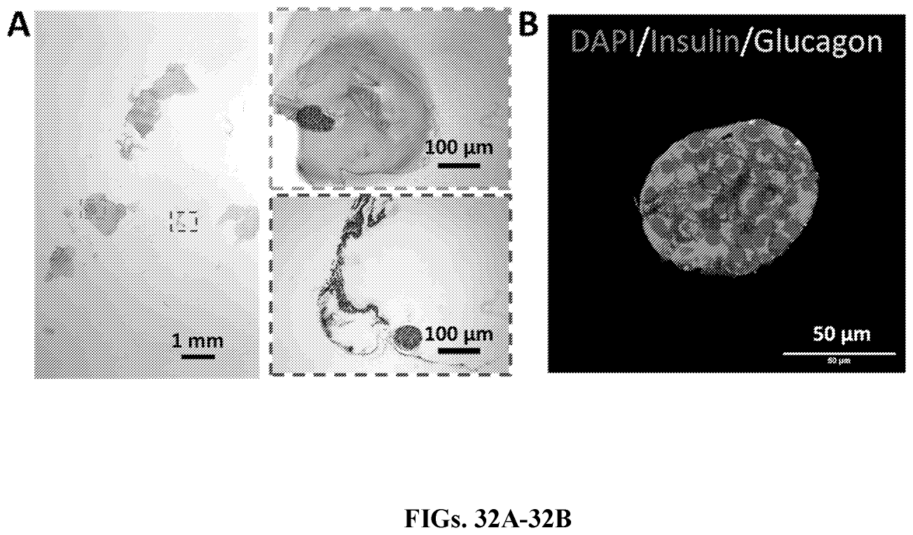

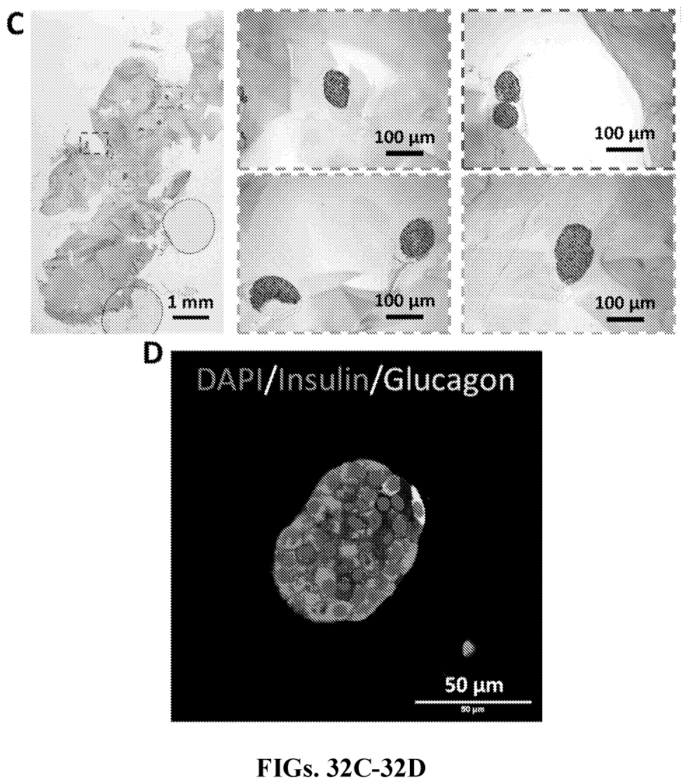

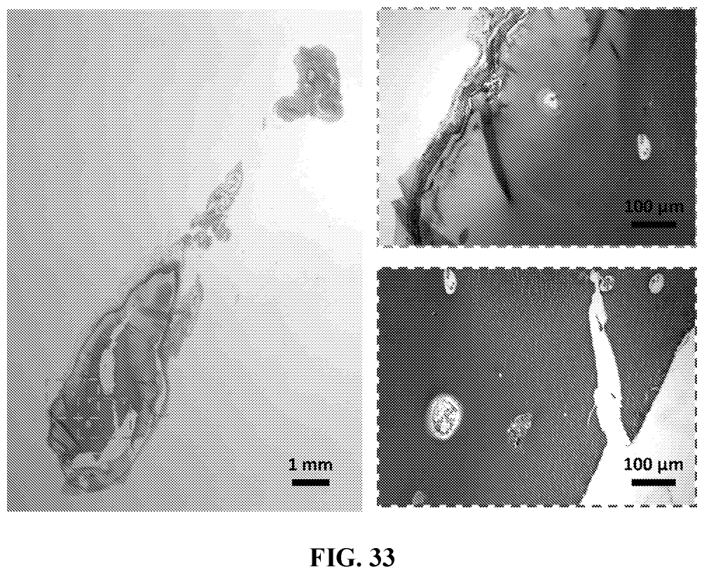

Following retrieval at 92 d, stereo microscope imaging showed that islets appeared as yellow with maintained smooth and intact morphology at all distances from the terminal tank, suggesting that islet health was preserved (FIG. 7N). Incubation of the device in 5% CO₂ buffer resulted in the formation of bubbles at the tubing surface, which indicated that O₂ production from the supply tank was maintained at this time point as well (FIGS. 70-7P and FIG. 3). H&E-stained slides and insulin/glucagon immunostaining confirmed that robust islets were observed at locations proximal, intermediate, and distal from the tank (FIGS. 7Q-7R), as well as in both superficial (FIG. 31) and central (FIG. 32) sections. On the other hand, control islets were largely fragmented and necrosed (FIG. 33). These results demonstrated the improved, long-term performance of the optimized iBEDv3.

### Example 7—Improved Cell Survival in Pigs

We next pursued an exploratory large animal study involving xenotransplantation of rat islets in Gottingen minipigs (FIG. 2). The SC space of pigs is anatomically similar to that of humans (Zheng et al., MAbs (Taylor & Francis), Vol. 4:243-255 (2012), which is hereby incorporated by reference in its entirety), thus this transplantation model provided the opportunity to study the potential for clinical translation. Additionally, the xenogeneic transplantation may exacerbate immune cell infiltration and likewise fibrotic deposition near the device and thus worsen graft oxygenation. It was hypothesized that enhanced O₂ supply was especially critical in this transplantation model. The primary intent of this proof-of-concept investigation was to derive a clinically feasible transplantation procedure for the iBED design and to study the potential for iBED to support surviving, functional cells in large animals.

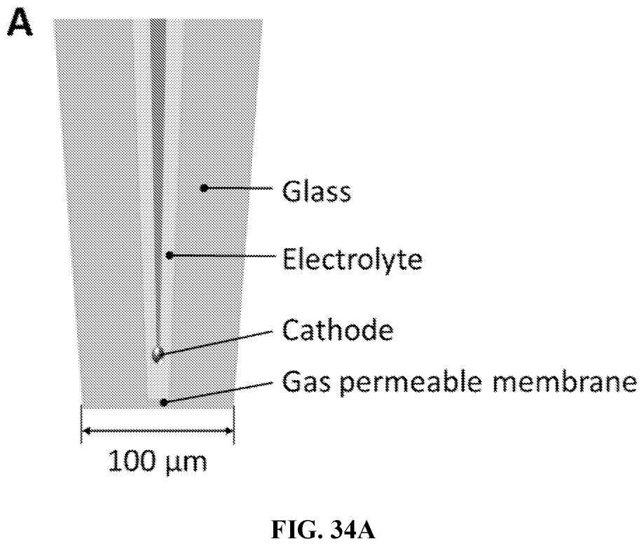

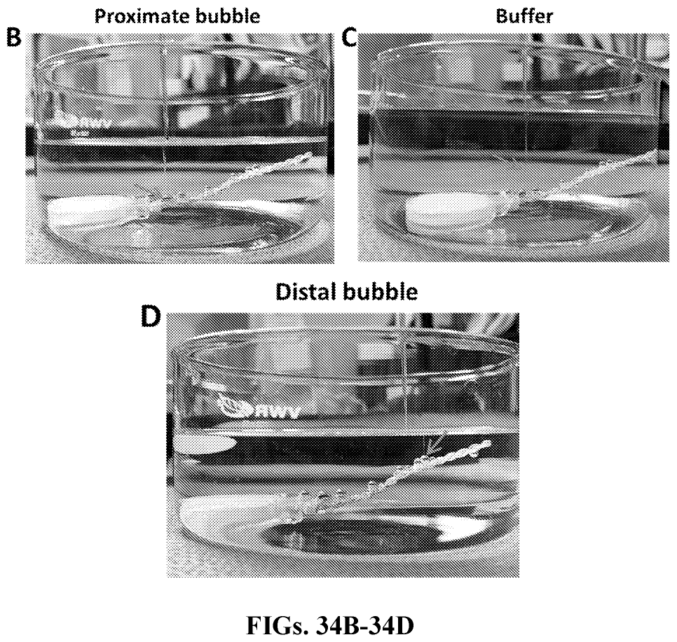

First, the iBEDv3 was modified and scaled to support higher islet payloads (iBEDv3S). The twisted silicone tubing was fixed in the adapter as previously described, though, in this design, the silicone tubing was only half-filled with PDMS prior to curing, providing protection from mechanical stress to alginate near the tank (FIG. 2A). In addition, the tank was enlarged to 22 mm diameter and 8 mm thickness, increasing the Li₂O₂/PFC loading capacity 10-fold from the iBEDv3 design, and the tubing length was extended to 40 mm (FIG. 2B). Following incubation in 5% CO₂, bubbles with high and uniform O₂ content were found at all lengths of the tubing, suggesting maintained CO₂-responsiveness and rapid gas transfer throughout the device (FIGS. 2C-2D and 34). The islet alginate encapsulation layer was added as done previously (FIG. 2E).

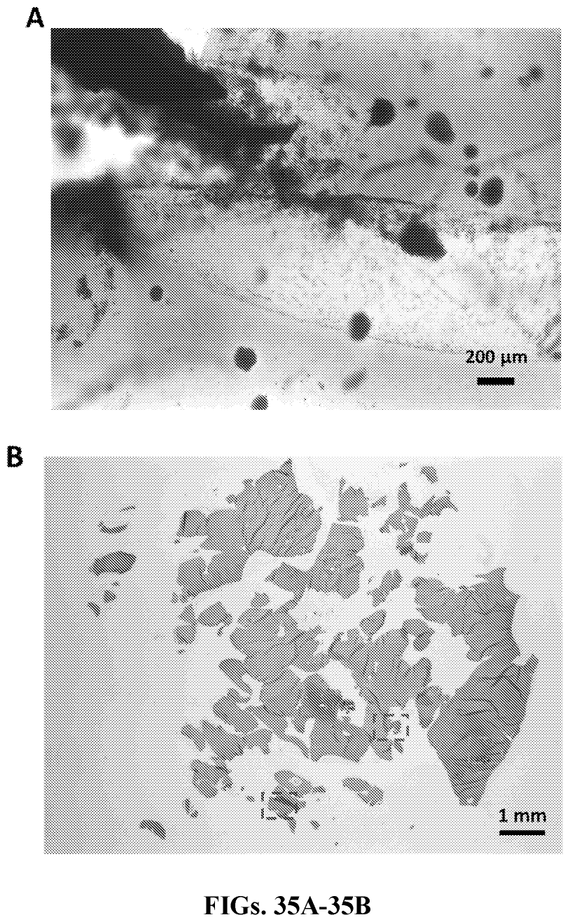

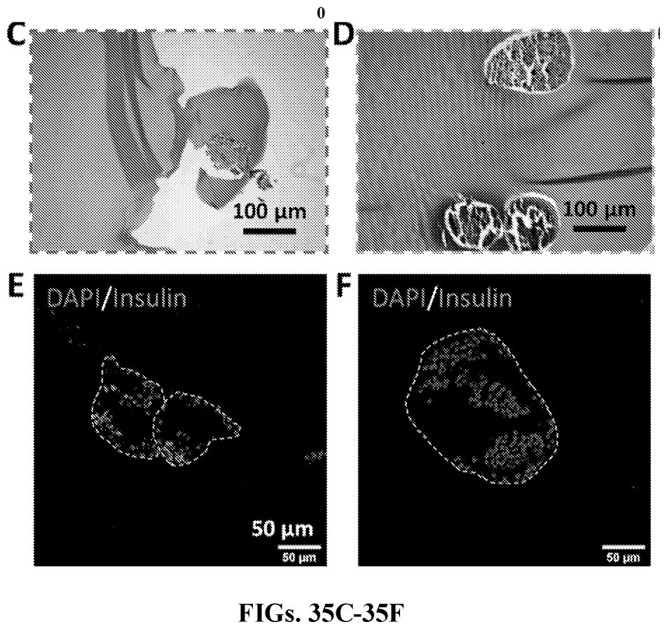

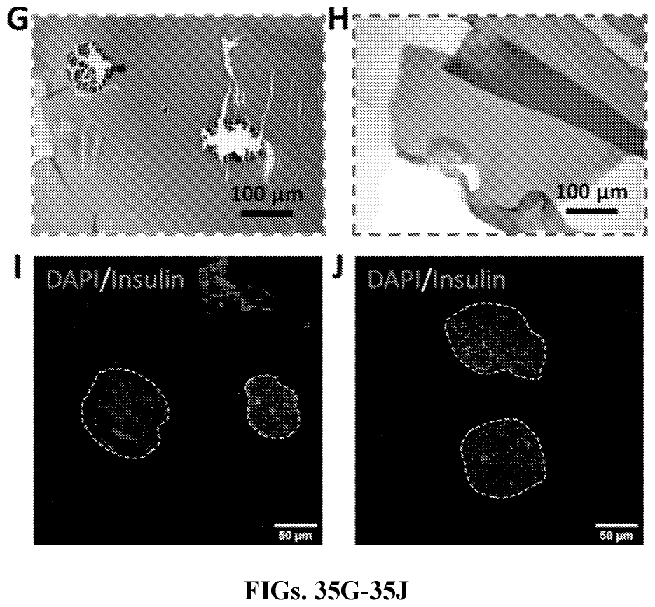

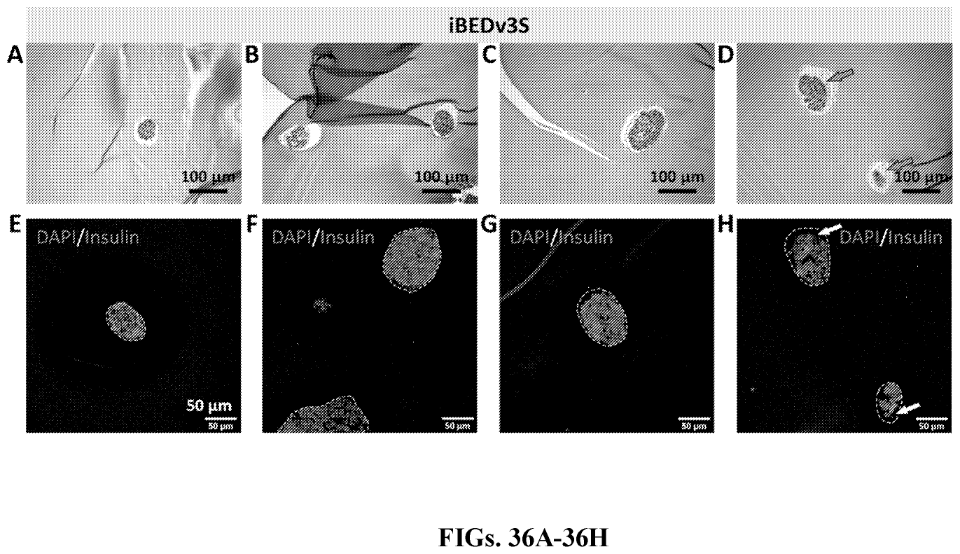

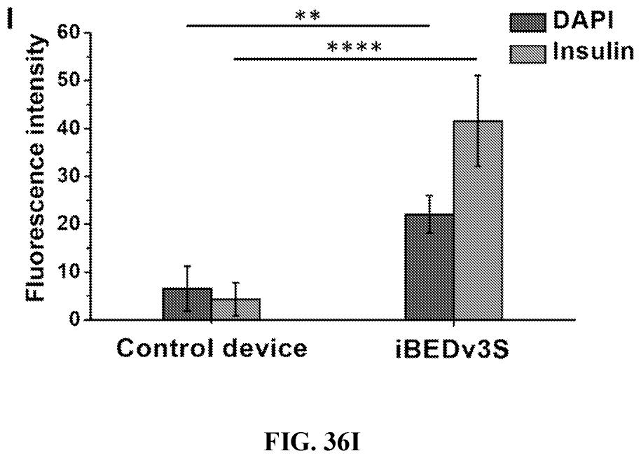

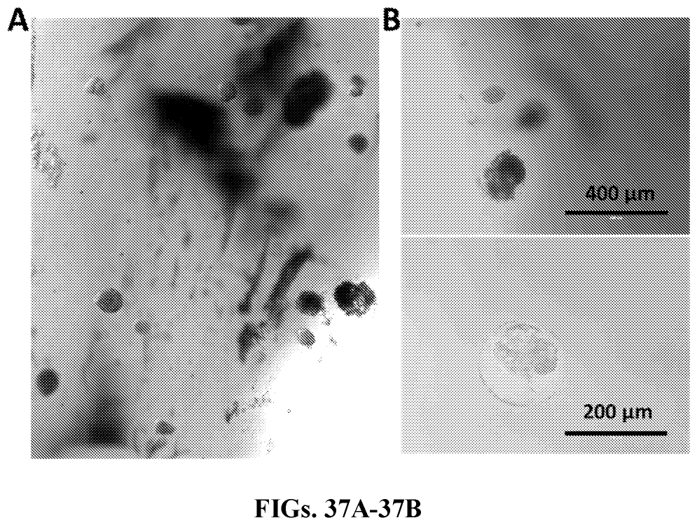

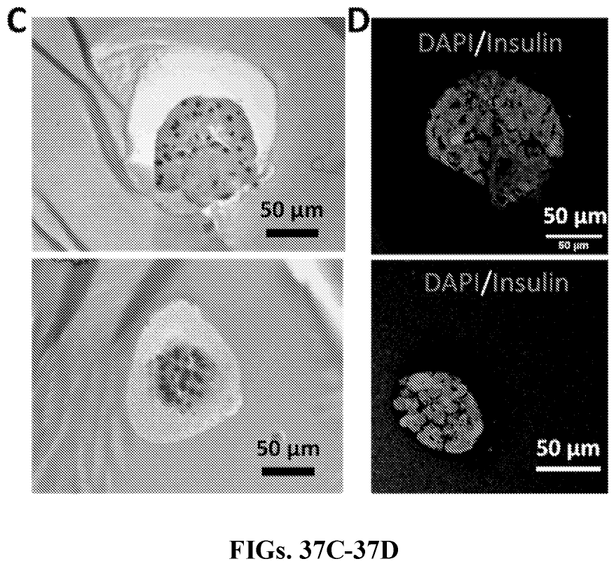

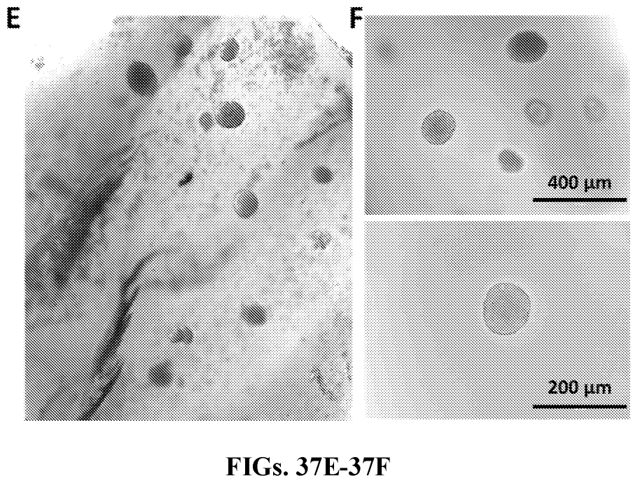

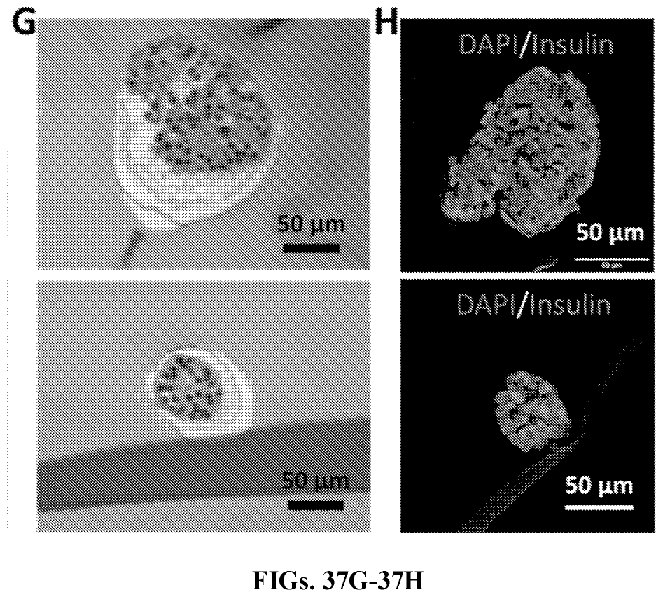

iBEDv3S devices, containing a subclinical dose of islets (1500 IEQ within 180 μL of alginate), were implanted in the ventral deep SC space in minipigs (n=4, including 3 minipigs for 1 month and 1 minipig for 2 months) (FIG. 2F). For controls, similarly constructed devices without the inverse-breathing mechanism were implanted lateral to the iBEDv3S devices in two of the pigs. Following retrieval at 1 month, almost all islets in the control devices were fragmented or necrosed with observable pyknosis, karyorrhexis, and loss of nuclei, and only weakly positive for insulin in certain regions (FIG. 35), indicating the challenging nature of the SC space for cell survival. In contrast, within retrieved iBEDv3S at 1 month, numerous surviving islets were observed with healthy morphology at both proximal and distal locations from the terminal tank (FIG. 2G). Apoptosis, when detected, was found in a few peripheral islet cells (FIGS. 36D-36H), rather than in central cells, suggesting that these cells might have died from a xenogeneic immune response rather than hypoxia. Immunostaining also revealed positive expression of insulin in retrieved islets, indicating maintained islet function (FIG. 2H and FIGS. 36E-36H). For comparison, insulin expression was 9.6-fold higher and DAPI content was 3.4-fold higher in islets from the iBEDv3S devices than that from the control devices (FIG. 36I). Characterization of the retrieved iBEDv3S at 2 months also revealed several intact and insulin-positive islets (though a higher portion of cells, again in the islet peripheries, exhibited apoptosis) (FIG. S37). These data demonstrate that the iBEDv3S was able to significantly improve cell survival and function in a xenogeneic SC transplantation in large animals.

### Discussion of Examples 1-7

Despite several decades of laboratory and clinical investigation, widespread clinical translation of islet encapsulation technology has not been realized in part due to O₂ limitations. Additional O₂ supply is critical for supporting islet survival and function and for permitting surgically realistic device volumes. Several thousand functional IEQ per patient kilogram are required to restore normoglycemia in a human patient (Papas et al., “Prediction of Marginal Mass Required for Successful Islet Transplantation,” J. Invest. Surg. 23:28-34 (2010), which is hereby incorporated by reference in its entirety) and in the absence of O₂ supplementation, they must be widely dispersed, even in thin constructs, to preserve O₂ availability (Lewis, “Eliminating Oxygen Supply Limitations for Transplanted Microencapsulated Islets in the Treatment of Type 1 Diabetes,” Thesis, Massachusetts Institute of Technology (2008) and Avgoustiniatos et al., “Measurements of the Effective Diffusion Coefficient of Oxygen in Pancreatic Islets,” Ind. Eng. Chem. Res. 46:6157-6163 (2007), which are hereby incorporated by reference in their entirety). This results in unfeasibly large estimated device sizes, on the order of meters for a cylindrical geometry, required to deliver a metabolically relevant payload (Dulong and Legallais., “A Theoretical Study of Oxygen Transfer Including Cell Necrosis for the Design of a Bioartificial Pancreas,” Biotechnol. Bioeng. 96:990-998 (2007), which is hereby incorporated by reference in its entirety). Enhanced O₂ supply allows islets to be encapsulated at higher densities without sacrificing O₂ availability thus reducing the required graft volume to a manageable level surgically.

While several O₂ supplementation approaches have been reported previously, all of which have demonstrated benefit to encapsulated cells, challenges remain. For example, the PAir device (Beta-O2) provides supraphysiological pO₂ levels via daily injections into a gas-permeable chamber (Barkai et al., “Enhanced Oxygen Supply Improves Islet Viability in a New Bioartificial Pancreas,” Cell Transplant. 22:1463-1476 (2013) and Carlsson et al., “Transplantation of Macroencapsulated Human Islets Within the Bioartificial Pancreas Bair to Patients with Type 1 Diabetes Mellitus,” Am. J. Transplantation 18:1735-1744 (2018), which are hereby incorporated by reference in their entirety). This device, however, requires daily purging and refilling, otherwise irreversible graft failure occurs rapidly (Barkai et al., “Enhanced Oxygen Supply Improves Islet Viability in a New Bioartificial Pancreas,” Cell Transplant. 22:1463-1476 (2013), which is hereby incorporated by reference in its entirety). O₂ delivery by electrolysis has also been explored, although application in vivo has not yet been reported (Wu et al., “In Situ Electrochemical Oxygen Generation with an Immunoisolation Device,” Ann. N. Y. Acad. Sci. 875:105-125 (1999), which is hereby incorporated by reference in its entirety). In addition, excess hydrogen produced at the cathode diffuses into the host tissue, which may not be adequately cleared and thus present a potential problem at the transplantation site. The CaO₂-containing constructs provide O₂ supply via a hydrolytic chemical reaction (Oh et al., “Oxygen Generating Scaffolds for Enhancing Engineered Tissue Survival,” Biomaterials 30:757-762 (2009); Pedraza et al., “Preventing Hypoxia-induced Cell Death in Beta Cells and Islets Via Hydrolytically Activated, Oxygen-Generating Biomaterials,” Proc. Natl. Acad. Sci. U.S.A. 109:4245-4250 (2012); and Coronel et al., “Oxygen Generating Biomaterial Improves the Function and Efficacy of Beta Cells Within a Macroencapsulation Device,” Biomaterials 210:1-11 (2019), which are hereby incorporated by reference in their entirety). However, as explained earlier, water is a suboptimal reactant for in vivo O₂ generation.

The inverse-breathing system of the device described herein provides an alternative solution to O₂ supplementation that could overcome many challenges discussed above. Here, O₂ was produced for the cells by their own waste product, CO₂, which is ubiquitous in tissues and is self-regulated, unlike water. As a result, the steady, self-controlled CO₂ levels facilitated the continuous release of O₂ over several months. Further, O₂ production (in the terminal tank) was physically separated from the encapsulated cells, which avoided any harmful impact of the O₂ generation process (e.g. pH, side products, and temperature change) on the cells. The iBED system combined the advantages of four unique properties of its constituent materials. Beyond the benefit of CO₂ responsiveness, Li₂O₂ has the highest O₂ content of all inorganic peroxides (˜33% w/w, versus ˜16% w/w for CaO₂, considering commercial purities) and thus can supply the highest amount of O₂ per unit weight. This potential was maximized by immersing the particulates in PFC, which has the distinctive capacity to resist water and dissolve high quantities of O₂ and CO₂ (Riess., “Fluorocarbon-based in Vivo Oxygen Transport and Delivery Systems,” Vox Sang. 61:225-239 (1991), which is hereby incorporated by reference in its entirety). Furthermore, the high gas permeability (and solid/liquid impermeability) of silicone (Merkel et al., “Gas Sorption, Diffusion, and Permeation in Poly (Dimethylsiloxane),” J. Polym. Sci., Part B: Polym. Phys. 38:415-434 (2000), which is hereby incorporated by reference in its entirety), and rapid gas-phase CO₂ and O₂ diffusion in air ensured rapid O₂ delivery despite the physical separation between the cells and the O₂ generating reaction (O'Brien et al., “Diffusion Coefficients of Respiratory Gases in a Perfluorocarbon Liquid,” Science: 153-155 (1982) and Chapman et al., “The Mathematical Theory of Non-Uniform Gases: An Account of the Kinetic Theory of Viscosity, Thermal Conduction and Diffusion in Gases,” Cambridge university press (1990), which are hereby incorporated by reference in their entirety). The advantages of these features contributed in concert to maximize the performance of the inverse-breathing system.

There are a few outstanding considerations concerning the clinical translation of this device. For example, while no adverse reactions were observed to the tank material in this study (FIG. 22C) or in a previous work (An et al., “An Atmosphere-Breathing Refillable Biphasic Device for Cell Replacement Therapy,” Adv. Mater. 31:1905135 (2019), which is hereby incorporated by reference in its entirety), it may not meet regulatory standards for long-term implantation in humans. However, this component can be also fabricated from clinically approved materials such as titanium. The use of the printable resin in the studies herein simply provided convenience necessary for prototyping.

Furthermore, although the physical containment of Li₂O₂ would obviate concerns of their potential toxicity, the prevention of mechanical failure is critical for translation and a perfectly safe device like the implantable biomedical pacemaker with safely isolated lithium component (Mulpuru et al., “Cardiac Pacemakers: Function, Troubleshooting, and Management: Part 1 of a 2-Part Series,” J. Am. Coll. Cardiol. 69:189-210 (2017), which is hereby incorporated by reference in its entirety) should be pursued in the future.

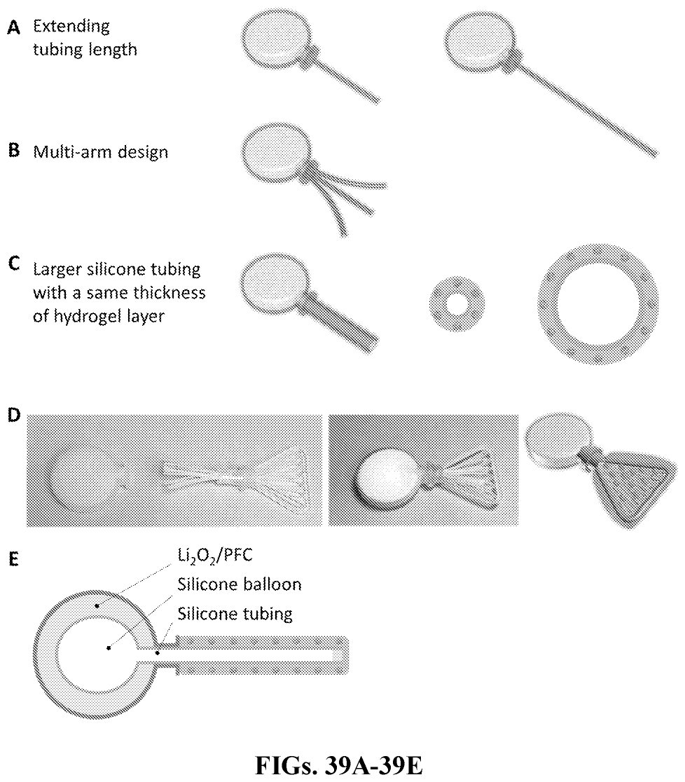

In the devices exemplified herein, O₂ supply is finite. While only 3 g of Li₂O₂ are required to sustain 500 k IEQ of human islets (a standard approximation of the dosage used in clinical islet transplantation) for over one month (Table 1), device replacement is impractical. However, O₂ supply may be extended indefinitely by the introduction of a tank replacement (FIG. 38A) or formulation refilling (FIG. 38B) system. There is also the question of scaling the device to support clinically relevant islet volumes. This may be accomplished by extending the tubing length (FIG. 39A), implementing a multi-arm design (FIG. 39B), or using a silicone tubing with a larger diameter (FIG. 39C). A planar prototype with multiple aeration channels connected to the terminal tank was also fabricated to this aim (FIG. 39D). Finally, the designs to increase silicone surface area within the terminal tank, such as incorporating a silicone balloon within the terminal tank, which should increase the O₂ production rate from the tank to support higher cell loads are contemplated (FIG. 39E).

TABLE 1
O₂ content of Li₂O₂ compared to O₂ demand of rat and human islets.
In the inverse-breathing reaction, 1 mg of Li₂O₂ yields 0.348 mg O₂.
Considering the maximum O₂ consumption rate of rat islets, 50 mg of Li₂O₂
should support 500 IEQ rat islets for 210 d, assuming all O₂ is consumed by the cells.
Under this assumption, and considering the O₂ consumption rate of human islets, 3 g Li₂O₂ should
support 500 k IEQ human islets for 33 d. Note, this comparison does not consider physical transport
phenomena such as diffusion, leakage, and O₂ supply from the host.

| | | Rat islets | 50 mg Li₂O₂ | Human islets | 3 g Li₂O₂ |
|---|---|---|---|---|---|
| 2Li₂O₂ + 2CO₂ → 2Li₂CO₃ + O₂ | | Maximum oxygen | 500 IEQ | Maximum oxygen | 500 k IEQ |
| Li₂O₂ | O₂ | consumption rate | rat islets | consumption rate | human islets |
| 1 mg | 0.348 mg | 0.034 mol/m³/s | 210 days | 0.013 mol/m³/s | 33 days |
| 0.0218 mmol | 0.0109 mmol | 6.01E−14 mol/IEQ/s | | 2.3E−14 mol/IEQ/s | |

Herein, the iBED system was extensively tested in vitro and in vivo. Initially, a simple inverse-breathing construct improved cell survival in hypoxic culture in vitro and in vivo, validating the efficacy of physiological CO₂-regulated O₂ production for encapsulated cells. Thereafter, a first-generation device (iBEDv1) improved cell survival and metabolic function following SC transplantation in mice for two months. Following a series of model-guided design optimizations, a third-generation device (iBEDv3) achieved sustained O₂ supply and diabetes correction in mice for 3 months, about 10 times longer than the non-oxygenated control, following transplantation in the SC site. Finally, a scaled third-generation device (iBEDv3S) was designed and implanted in the SC space of minipigs. Even in the xenogeneic environment (which may be more challenging than clinical human-to-human allotransplantation), numerous surviving and functional islets were found following retrieval at one and two months. These findings show substantial progress in the translation of in situ long-term O₂-supplementation systems for encapsulated islets.

In this work, the design, characterization, and testing of a novel system and device was presented to overcome limitations of O₂ supplementation in cell replacement therapies. Critically, O₂ production in iBED was regulated by CO₂, a waste product of the encapsulated cells and cells in the surrounding tissues. This key self-regulation feature enabled the sustained delivery of O₂ for several months without intervention. The inverse-breathing system presented here provides a novel solution to many problems of supplying O₂ to encapsulated cells and represents a self-sustaining technology well-suited for realizing clinical translation of cell replacement therapies in the SC site.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A reservoir device for providing oxygen to encapsulated cells, the device comprising:
a reservoir suitable for enclosing a liquid;
a liquid contained within the reservoir, wherein the liquid is permeable to gas; and
an oxygen-generating compound, wherein said oxygen-generating compound is immersed in the liquid contained within the reservoir.

2. The reservoir device of claim 1, wherein the reservoir further comprises an opening.

3. The reservoir device of claim 2, wherein the opening further comprises a fitting.

4. The reservoir device of claim 2 or claim 3, wherein the opening is capped with a gas permeable membrane.

5. The reservoir device of claim 4, wherein the gas permeable membrane is permeable to oxygen and carbon dioxide.

6. The reservoir device of claim 4 or claim 5, wherein the gas permeable membrane comprises a material selected from the group consisting of polydimethylsiloxane (PDMS), silicone-based membranes, and polytetrafluoroethylene (PTFE).

7. The reservoir device of any one of claims 1-6, wherein the oxygen-generating compound reacts with carbon dioxide to release oxygen.

8. The reservoir device of any one of claims 1-7, wherein the oxygen-generating compound comprises lithium peroxide, sodium peroxide, potassium peroxide, potassium superoxide, and combinations thereof.

9. The reservoir device of any one of claim 1-8, wherein the liquid of the reservoir is selected from the group consisting of perfluorocarbon (PFC) oil, mineral oil, silicone oil, and combinations thereof.

10. The reservoir device of any one of claims 1-9, wherein the reservoir comprises a wall enclosing the liquid.

11. The reservoir device of claim 10, wherein the wall is impermeable to solids, liquids, and gases.

12. The reservoir device of claim 10 or claim 11, wherein the wall comprises a biocompatible material.

13. The reservoir device of claim 12, wherein the wall comprises a biocompatible resin, a medical grade alloy, titanium, titanium alloy, stainless steel, cobalt chrome alloy, nickel titanium alloy, gold, platinum, silver, iridium, tantalum, tungsten, and combinations thereof.

14. The reservoir device of any one of claims 1-13, wherein the device comprises a flattened configuration, a cylindrical configuration, a rectangular configuration or combinations thereof.

15. The reservoir device of any one of claims 1-14, wherein the device is configured for subcutaneous placement in a subject, preperitoneal placement in a subject, transperitoneal placement in a subject, transcutaneous placement in a subject, and/or intraperitoneal placement in a subject.

16. The reservoir device of any one of claims 1-15, wherein the reservoir comprises a plurality of openings.

17. The reservoir device of any one of claims 1-16, wherein the reservoir has the dimensions of 10-80 mm diameter and 3-30 mm height.

18. The reservoir device of any one of claims 1-17, wherein the reservoir is configured to be refillable with oxygen-generating compound and/or liquid.

19. A cell encapsulation device comprising:
a gas permeable membrane having proximal and distal ends, said gas permeable membrane enclosing an inner space that extends longitudinally between the proximal and distal ends of the gas permeable membrane, wherein the inner space is at least partially filled with air; and
a hydrogel layer covering an outer surface of the gas permeable membrane.

20. The encapsulation device of claim 19, wherein the gas permeable membrane further comprises an opening at the proximal or distal end.

21. The encapsulation device of claim 20, wherein the opening further comprises a fitting.

22. The encapsulation device claim 20 or claim 21, wherein the opening is capped with a gas permeable fitting.

23. The encapsulation device of claim 22, wherein the gas permeable fitting is permeable to oxygen and carbon dioxide.

24. The encapsulation device of claim 22 or claim 23, wherein the gas permeable fitting comprises a material selected from the group consisting of polydimethylsiloxane (PDMS), silicone-based membranes, and polytetrafluoroethylene (PTFE).

25. The encapsulation device of any one of claims 19-24, wherein the hydrogel layer comprises a material selected from the group consisting of alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative, and combinations thereof.

26. The encapsulation device of any one of claims 19-25, wherein the hydrogel layer comprises a thickness of 200-2000 μm.

27. The encapsulation device of any one of claims 19-26, wherein the outer surface of the gas permeable membrane is modified to accept the hydrogel layer.

28. The encapsulation device of claim 27, wherein the outer surface of the gas permeable membrane is modified by salt leaching, a polymer coating, or both to accept the hydrogel layer.

29. The encapsulation device of any one of claims 19-28, wherein the hydrogel layer contains cells.

30. The encapsulation device of claim 29, wherein the cells comprise cells selected from the group consisting of islet cells, stem cell-derived β cells, Factor VIII-producing fibroblasts, hepatocytes, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof, and combinations thereof.

31. The encapsulation device of claim 30, wherein the cells produce one or more of insulin, coagulation factors, albumin, urea, human cytochrome P450 enzymes.

32. The encapsulation device of any one of claims 29-31, wherein the cells are present in the hydrogel layer of the device at a concentration of 1%-40% v/v cells/hydrogel.

33. The encapsulation device of any one of claims 19-32, wherein said device is coupled via the opening at its proximal or distal end to a reservoir device for providing oxygen to the cells of the hydrogel layer.

34. The encapsulation device of claim 33 wherein said reservoir device comprises:
a reservoir suitable for enclosing a liquid;
a liquid contained within the reservoir, wherein the liquid is permeable to gas; and
an oxygen-generating compound, wherein said oxygen-generating compound is immersed in the liquid contained within the reservoir.

35. The encapsulation device of claim 34, wherein the liquid of the reservoir is selected from the group consisting of perfluorocarbon (PFC) oil, mineral oil, silicone oil, and combinations thereof.

36. The encapsulation device of claim 34 or claim 35, wherein the oxygen-generating compound reacts with carbon dioxide to release oxygen.

37. The encapsulation device of any one of claims 33-35, wherein the oxygen-generating compound comprises lithium peroxide, sodium peroxide, potassium peroxide, potassium superoxide, and combinations thereof.

38. The encapsulation device of any one of claims 34-37, wherein the gas permeable membrane is permeable to oxygen and carbon dioxide

39. The encapsulation device of any one of claims 34-38, wherein the reservoir comprises a wall enclosing the liquid.

40. The encapsulation device of claim 39, wherein the wall is impermeable to solids, liquids, and gases.

41. The encapsulation device of claim 39 or claim 40, wherein the wall comprises a biocompatible material.

42. The encapsulation device of any one of claims 39-41, wherein the wall comprises a biocompatible resin, a medical grade alloy, titanium, titanium alloy, stainless steel, cobalt chrome alloy, nickel titanium alloy, gold, platinum, silver, iridium, tantalum, tungsten, and combinations thereof.

43. The encapsulation device of any one of claims 34-42, wherein the device has the dimensions of 10-80 mm diameter and 3-30 mm height.

44. The encapsulation device of any one of claims 19-43, wherein the gas permeable membrane comprises a material selected from the group consisting of polydimethylsiloxane (PDMS), silicone-based membranes, and polytetrafluoroethylene (PTFE).

45. The encapsulation device of any one of claims 19-44, wherein the encapsulation device comprises an elongated configuration, a flattened configuration, a cylindrical configuration, a rectangular configuration, or combinations thereof

46. The encapsulation device of any one of claims 19-45, wherein the encapsulation device is configured for subcutaneous placement, transcutaneous placement, preperitoneal placement, transperitoneal placement, or intraperitoneal placement in a subject

47. The encapsulation device of any one of claims 19-46, wherein the device comprises a plurality of openings.

48. A cell encapsulation device comprising:
two or more elongated gas permeable membranes, each membrane comprising proximal and distal ends and enclosing an inner space that extends longitudinally between the proximal and distal ends of the gas permeable membrane, wherein the inner space enclosed by each membrane is at least partially filled with air, wherein the proximal ends of the membranes comprise an opening and the distal ends of the membranes are sealed, and wherein the two or more gas permeable membranes are twisted helically about a longest axis shared by the membranes; and
a hydrogel layer covering outer surfaces of the two or more helically twisted gas permeable membranes.

49. The encapsulation device of claim 48, further comprising fittings, wherein the openings at the proximal end of the membranes are each connected to a fitting.

50. The encapsulation device of claim 49, wherein the fittings are permeable to oxygen and carbon dioxide.

51. The encapsulation device of claim 49 or 50, wherein the fittings comprise a material selected from the group consisting of polydimethylsiloxane (PDMS), silicone-based membranes, and polytetrafluoroethylene (PTFE).

52. The encapsulation device of any one of claims 48-51, wherein the hydrogel layer comprises a material selected from the group consisting of alginate, collagen, hyaluronate, fibrin, fibroin, agarose, chitosan, bacterial cellulose, elastin, keratin, polyethylene glycol, a polyethylene glycol derivative, poly(2-hydroxyethyl methacrylate), a poly(2-hydroxyethyl methacrylate) derivative, and combinations thereof.

53. The encapsulation device of any one of claims 48-52, wherein the hydrogel layer comprises a thickness of 200-2000 μm.

54. The encapsulation device of any one of claims 48-53, wherein the outer surfaces of the gas permeable membranes are modified to accept the hydrogel layer.

55. The encapsulation device of claim 54, wherein the outer surfaces of the gas permeable membranes are modified by salt leaching, a polymer coating, or both to accept the hydrogel layer.

56. The encapsulation device of any one of claims 48-55, wherein the hydrogel layer contains cells.

57. The encapsulation device of claim 56, wherein the cells are selected from the group consisting of islet cells, stem cell-derived β cells, Factor VIII-producing fibroblasts, hepatocytes, endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof, and combinations thereof

58. The encapsulation device of claim 57, wherein the cells produce one or more of insulin, coagulation factors, albumin, urea, human cytochrome P450 enzymes

59. The encapsulation device of any one of claims 56-58, wherein the cells are present in the hydrogel layer of the device at a concentration of 1%-40% v/v cells/hydrogel.

60. The encapsulation device of any one of claims 48-59, wherein the device further comprises a plurality of elongated gas permeable membranes.

61. The encapsulation device of any one of claims 48-60, wherein said device is coupled via the openings at the proximal ends of the gas permeable membranes to a reservoir device for providing oxygen to the cells of the hydrogel layer.

62. The encapsulation device of claim 61, wherein said reservoir device comprises:
a reservoir suitable for enclosing a liquid;
a liquid contained within the reservoir, wherein the liquid is permeable to gas; and
an oxygen-generating compound, wherein said oxygen-generating compound is immersed in the liquid contained within the reservoir.

63. The encapsulation device of claim 61, wherein the liquid of the reservoir is selected from the group consisting of perfluorocarbon (PFC) oil, mineral oil, silicone oil, and combinations thereof.

64. The encapsulation device of claim 62 or claim 63, wherein the oxygen-generating compound reacts with carbon dioxide to release oxygen.

65. The encapsulation device of any one of claims 62-64, wherein the oxygen-generating compound comprises lithium peroxide, sodium peroxide, potassium peroxide, potassium superoxide, and combinations thereof.

66. The encapsulation device of any one of claims 62-65, wherein the gas permeable membrane is permeable to oxygen and carbon dioxide

67. The encapsulation device of any one of claims 62-66, wherein the reservoir comprises a wall enclosing the liquid.

68. The encapsulation device of claim 67, wherein the wall is impermeable to solids, liquids, and gases.

69. The encapsulation device of claim 67 or claim 68, wherein the wall comprises a biocompatible material.

70. The encapsulation device of any one of claims 66-69, wherein the wall comprises a biocompatible resin, a medical grade alloy, titanium, titanium alloy, stainless steel, cobalt chrome alloy, nickel titanium alloy, gold, platinum, silver, iridium, tantalum, tungsten, and combinations thereof.

71. The encapsulation device of any one of claims 69-70, wherein the device has the dimensions of 10-80 mm diameter and 3-30 mm height.

72. The encapsulation device of any one of claims 69-71, wherein the gas permeable membrane comprises a material selected from the group consisting of polydimethylsiloxane (PDMS), silicone-based membranes, and polytetrafluoroethylene (PTFE).

73. The encapsulation device of any one of claims 48-72, wherein the device is configured for subcutaneous placement, transcutaneous placement, preperitoneal placement, transperitoneal placement, or intraperitoneal placement in a subject.

74. A system for providing oxygen to encapsulated cells, the system comprising:
the reservoir device of any one of claims 1-18; and
the cell encapsulation device of any one of claims 19-73.

75. The system of claim 74, wherein the reservoir device is in fluid connection with the encapsulation device.

76. The system of claim 74, wherein the reservoir device is in gas connection with the encapsulation device.

77. The system of any one of claims 74-76, wherein the encapsulation device is coupled to the reservoir device with a gas permeable fitting.

78. A method of delivering a therapeutic agent to a subject in need thereof, the method comprising:
obtaining a cell encapsulation device of any one of claims 19-73 or system of claims 74-77; and
implanting the encapsulation device or system in the subject.

79. The method of claim 78, wherein the subject is selected from the group consisting of a human, a mouse, a rat, a dog, a pig, a sheep, a cow, or a nonhuman primate.

80. The method of claim 78 or claim 79, wherein the encapsulation device or system is implanted subcutaneously.

81. The method of any one of claims 78-80, wherein the subject suffers from diabetes.

82. The method of any one of claims 78-81, wherein the encapsulation device or system comprises insulin-producing cells and said device or system provides insulin to the subject.

83. The method of claim 81 or claim 82, wherein the encapsulation device or system provides insulin to said subject for at least 15, 30, 45, 60, 75, or 90 days.

84. The method of any one of claims 78-83 further comprising:
retrieving one or more components of the implanted encapsulation device or system from the subject.

85. The method of claim 84 further comprising:
replacing the retrieved component of the implanted encapsulation device or system from the subject.
